SEQUENCE LISTING (102 sequences)

SEQ ID NO: 1 - protein, 6 aa, Homo sapiens
MYPPPY

SEQ ID NO: 2 - protein, 4 aa, Homo sapiens
SICT

SEQ ID NO: 3 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 4 - DNA, 1997 bp, Homo sapiens
gctttctattcaagtgccttctgtgtgtgcacatgtgtaatacatatctgggatcaaagctatctatataaagtccttgattctgtgtgggttcaaacacatttcaaagcttcaggatcctgaaaggttttgctctacttcctgaagacctgaacaccgctcccataaagccatggcttgccttggatttcagcggcacaaggctcagctgaacctggctaccaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgacttcctcctctggatccttgcagcagttagttcggggttgtttttttatagctttctcctcacagctgtttctttgagcaaaatgctaaagaaaagaagccctcttacaacaggggtctatgtgaaaatgcccccaacagagccagaatgtgaaaagcaatttcagccttattttattcccatcaattgagaaaccattatgaagaagagagtccatatttcaatttccaagagctgaggcaattctaacttttttgctatccagctatttttatttgtttgtgcatttggggggaattcatctctctttaatataaagttggatgcggaacccaaattacgtgtactacaatttaaagcaaaggagtagaaagacagagctgggatgtttctgtcacatcagctccactttcagtgaaagcatcacttgggattaatatggggatgcagcattatgatgtgggtcaaggaattaagttagggaatggcacagcccaaagaaggaaaaggcagggagcgagggagaagactatattgtacacaccttatatttacgtatgagacgtttatagccgaaatgatcttttcaagttaaattttatgccttttatttcttaaacaaatgtatgattacatcaaggcttcaaaaatactcacatggctatgttttagccagtgatgctaaaggttgtattgcatatatacatatatatatatatatatatatatatatatatatatatatatatatatatatatattttaatttgatagtattgtgcatagagccacgtatgtttttgtgtatttgttaatggtttgaatataaacactatatggcagtgtctttccaccttgggtcccagggaagttttgtggaggagctcaggacactaatacaccaggtagaacacaaggtcatttgctaactagcttggaaactggatgaggtcatagcagtgcttgattgcgtggaattgtgctgagttggtgttgacatgtgctttggggcttttacaccagttcctttcaatggtttgcaaggaagccacagctggtggtatctgagttgacttgacagaacactgtcttgaagacaatggcttactccaggagacccacaggtatgaccttctaggaagctccagttcgatgggcccaattcttacaaacatgtggttaatgccatggacagaagaaggcagcaggtggcagaatggggtgcatgaaggtttctgaaaattaacactgcttgtgtttttaactcaatattttccatgaaaatgcaacaacatgtataatatttttaattaaataaaaatctgtggtggtcgttttccgga

SEQ ID NO: 5 - protein, 174 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIAKEKKPSYNRGLCENAPNRARM

SEQ ID NO: 6 - DNA, 1887 bp, Homo sapiens
gctttctattcaagtgccttctgtgtgtgcacatgtgtaatacatatctgggatcaaagctatctatataaagtccttgattctgtgtgggttcaaacacatttcaaagcttcaggatcctgaaaggttttgctctacttcctgaagacctgaacaccgctcccataaagccatggcttgccttggatttcagcggcacaaggctcagctgaacctggctaccaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgctaaagaaaagaagccctcttacaacaggggtctatgtgaaaatgcccccaacagagccagaatgtgaaaagcaatttcagccttattttattcccatcaattgagaaaccattatgaagaagagagtccatatttcaatttccaagagctgaggcaattctaacttttttgctatccagctatttttatttgtttgtgcatttggggggaattcatctctctttaatataaagttggatgcggaacccaaattacgtgtactacaatttaaagcaaaggagtagaaagacagagctgggatgtttctgtcacatcagctccactttcagtgaaagcatcacttgggattaatatggggatgcagcattatgatgtgggtcaaggaattaagttagggaatggcacagcccaaagaaggaaaaggcagggagcgagggagaagactatattgtacacaccttatatttacgtatgagacgtttatagccgaaatgatcttttcaagttaaattttatgccttttatttcttaaacaaatgtatgattacatcaaggcttcaaaaatactcacatggctatgttttagccagtgatgctaaaggttgtattgcatatatacatatatatatatatatatatatatatatatatatatatatatatatatatatatattttaatttgatagtattgtgcatagagccacgtatgtttttgtgtatttgttaatggtttgaatataaacactatatggcagtgtctttccaccttgggtcccagggaagttttgtggaggagctcaggacactaatacaccaggtagaacacaaggtcatttgctaactagcttggaaactggatgaggtcatagcagtgcttgattgcgtggaattgtgctgagttggtgttgacatgtgctttggggcttttacaccagttcctttcaatggtttgcaaggaagccacagctggtggtatctgagttgacttgacagaacactgtcttgaagacaatggcttactccaggagacccacaggtatgaccttctaggaagctccagttcgatgggcccaattcttacaaacatgtggttaatgccatggacagaagaaggcagcaggtggcagaatggggtgcatgaaggtttctgaaaattaacactgcttgtgtttttaactcaatattttccatgaaaatgcaacaacatgtataatatttttaattaaataaaaatctgtggtggtcgttttccgga

SEQ ID NO: 7 - protein, 223 aa, Mus musculus
MACLGLRRYKAQLQLPSRTWPFVALLTLLFIPVFSEAIQVTQPSVVLASSHGVASFPCEYSPSHNTDEVRVTVLRQTNDQMTEVCATTFTEKNTVGFLDYPFCSGTFNESRVNLTIQGLRAVDTGLYLCKVELMYPPPYFVGMGNGTQIYVIDPEPCPDSDFLLWILVAVSLGLFFYSFLVTAVSLSKMLKKRSPLTTGVYVKMPPTEPECEKQFQPYFIPIN

SEQ ID NO: 8 - DNA, 1933 bp, Mus musculus
ctacacatatgtagcacgtaccttggatcaaagctgtctatataaagtccccgagtctgtgtgggttcaaacacatctcaaggcttctggatcctgttgggttttactctgctccctgaggacctcagcacatttgccccccagccatggcttgtcttggactccggaggtacaaagctcaactgcagctgccttctaggacttggccttttgtagccctgctcactcttcttttcatcccagtcttctctgaagccatacaggtgacccaaccttcagtggtgttggctagcagccatggtgtcgccagctttccatgtgaatattcaccatcacacaacactgatgaggtccgggtgactgtgctgcggcagacaaatgaccaaatgactgaggtctgtgccacgacattcacagagaagaatacagtgggcttcctagattaccccttctgcagtggtacctttaatgaaagcagagtgaacctcaccatccaaggactgagagctgttgacacgggactgtacctctgcaaggtggaactcatgtacccaccgccatactttgtgggcatgggcaacgggacgcagatttatgtcattgatccagaaccatgcccggattctgacttcctcctttggatccttgtcgcagttagcttggggttgtttttttacagtttcctggtcactgctgtttctttgagcaagatgctaaagaaaagaagtcctcttacaacaggggtctatgtgaaaatgcccccaacagagccagaatgtgaaaagcaatttcagccttattttattcccatcaactgaaaggccgtttatgaagaagaaggagcatacttcagtctctaaaagctgaggcaatttcaactttccttttctctccagctatttttacctgtttgtatattttaaggagagtatgcctctctttaatagaaagctggatgcaaaattccaattaagcatactacaatttaaagctaaggagcatgaacagagagctgggatatttctgttgtgtcagaaccattttactaaaagcatcacttggaagcagcataaggatatagcattatggtgtggggtcaagggaacattagggaatggcacagcccaaagaaaggaagggggtgaaggaagagattatattgtacacatcttgtatttacctgagagatgtttatgacttaaataatttttaaatttttcatgctgttattttctttaacaatgtataattacacgaaggtttaaacatttattcacagagctatgtgacatagccagtggttccaaaggttgtagtgttccaagatgtatttttaagtaatattgtacatgggtgtttcatgtgctgttgtgtatttgctggtggtttgaatataaacactatgtatcagtgtcgtcccacagtgggtcctggggaggtttggctggggagcttaggacactaatccatcaggttggactcgaggtcctgcaccaactggcttggaaactagatgaggctgtcacagggctcagttgcataaaccgatggtgatggagtgtaaactgggtctttacactcattttattttttgtttctgcttttgttttcttcaatgatttgcaaggaaaccaaaagctggcagtgtttgtatgaacctgacagaacactgtcttcaaggaaatgcctcattcctgagaccagtaggtttgtttttttaggaagttccaatactaggaccccctacaagtactatggctcctcgaaaacacaaagttaatgccacaggaagcagcagatggtaggatgggatgcacaagagttcctgaaaactaacactgttagtgttttttttttaactcaatattttccatgaaaatgcaaccacatgtataatatttttaattaaataaaagtttcttgtgattgtttt

SEQ ID NO: 9 - protein, 142 aa, Artificial Sequence
  Description of Artificial Sequence: Synthetic polypeptide
MDVLVLFLCLVAFPSCVLSQVQLKESGPGLVAPSQSLSITCTVSGFSLTSYGVYWVRQPPGKGLEWLGVIWAGGTTNYNSALMSRLSISKDNSKSQVFLKMSSLQTDDTAMYYCARGPPHAMMKRGYAMDYWGQGTSVIVSS

SEQ ID NO: 10 - protein, 128 aa, Artificial Sequence
  Description of Artificial Sequence: Synthetic polypeptide
MDFQVQIFSFLLISASVILSRGQNVLTQSPAIMPASPGEKVTMTCSATSSITYMSWYQQKSGSSPRLLIYDTSNLASGVPVRFSGSGSGTSYSLTISRMEAEDAATYYCQQWSSYPLTFGAGTKLELK

SEQ ID NO: 11 - DNA, 384 bp, Artificial Sequence
  Description of Artificial Sequence: Synthetic polynucleotide
atggattttcaagtgcagattttcagcttcctgctaatcagtgcctcagtcatactgtccagaggacaaaatgttctcacccagtctccagcaatcatgcctgcatctccaggggagaaggtcaccatgacctgcagtgccacctcaagtataacttacatgtcctggtaccagcagaagtcaggatcctcccccagactcctgatttatgacacatccaacctggcttctggagtccctgttcgcttcagtggcagtgggtctgggacctcttactctctcacaatcagccgaatggaggctgaagatgctgccacttattactgccagcagtggagtagttacccgctcacgttcggtgctgggaccaagctggagctgaaa

SEQ ID NO: 12 - protein, 123 aa, Artificial Sequence
  Description of Artificial Sequence: Synthetic polypeptide
QVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSS

SEQ ID NO: 13 - DNA, 371 bp, Artificial Sequence
  Description of Artificial Sequence: Synthetic polynucleotide
caggtgcagctgcaagagtcaggacctggcctggtgaagccctcacagacactgtccttgacttgcactgtctctgggttttcattaacctcatatggtgtatattgggttcgccagcctccaggaaagggtctggagtggctgggagtaatatgggctggtggtaccacaaattataattcggctctcatgtccagactgacaatcagcaaagacacatccaagaaccaagtttccttaaaactcagcagtgtgactgcagcggacacagccgtctactactgtgcccgaggccccccgcacgctatgatgaagagaggctatgctatggactactggggacaaggaaccctagtcacagtctcctcagg

SEQ ID NO: 14 - protein, 106 aa, Artificial Sequence
  Description of Artificial Sequence: Synthetic polypeptide
DIQMTQSPSSLSASVGDRVTITCSATSSITYMSWYQQKPGKAPKLLIYDTSNLASGVPSRFSGSGSGTDYTLTISSLQPEDFATYYCQQWSSYPLTFGGGTKLEIK

SEQ ID NO: 15 - protein, 118 aa, Artificial Sequence
  Description of Artificial Sequence: Synthetic polypeptide
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 16 - DNA, 355 bp, Artificial Sequence
  Description of Artificial Sequence: Synthetic polynucleotide
caggtgcagctggtggagtctgggggaggcgtggtccagcctgggaggtccctgagactctcctgtgcagcctctggattcaccttcagtagctatactatgcactgggtccgccaggctccaggcaaggggctggagtgggtgacatttatatcatatgatggaaacaataaatactacgcagactccgtgaagggccgattcaccatctccagagacaattccaagaacacgctgtatctgcaaatgaacagcctgagagctgaggacacggctatatattactgtgcgaggaccggctggctggggccctttgactactggggccagggaaccctggtcaccgtctcctcag

SEQ ID NO: 17 - protein, 108 aa, Artificial Sequence
  Description of Artificial Sequence: Synthetic polypeptide
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 18 - DNA, 325 bp, Artificial Sequence
  Description of Artificial Sequence: Synthetic polynucleotide
gaaattgtgttgacgcagtctccaggcaccctgtctttgtctccaggggaaagagccaccctctcctgcagggccagtcagagtgttggcagcagctacttagcctggtaccagcagaaacctggccaggctcccaggctcctcatctatggtgcattcagcagggccactggcatcccagacaggttcagtggcagtgggtctgggacagacttcactctcaccatcagcagactggagcctgaagattttgcagtgtattactgtcagcagtatggtagctcaccgtggacgttcggccaagggaccaaggtggaaatcaaac

SEQ ID NO: 19 - protein, 241 aa, Artificial Sequence
  Description of Artificial Sequence: Synthetic polypeptide
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKGGGGSGGGGSGGGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 20 - protein, 244 aa, Artificial Sequence
  Description of Artificial Sequence: Synthetic polypeptide
DIQMTQSPSSLSASVGDRVTITCSATSSITYMSWYQQKPGKAPKLLIYDTSNLASGVPSRFSGSGSGTDYTLTISSLQPEDFATYYCQQWSSYPLTFGGGTKVEIKGGGGSGGGGSGGGGSQVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSS

SEQ ID NO: 21 - protein, 221 aa, Artificial Sequence
  Description of Artificial Sequence: Synthetic polypeptide
QVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 22 - protein, 213 aa, Artificial Sequence
  Description of Artificial Sequence: Synthetic polypeptide
DIQMTQSPSSLSASVGDRVTITCSATSSITYMSWYQQKPGKAPKLLIYDTSNLASGVPSRFSGSGSGTDYTLTISSLQPEDFATYYCQQWSSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 482 aa, Artificial Sequence
  Description of Artificial Sequence: Synthetic polypeptide
QVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSSGGSGGSGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKGGSGGSGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSGGSGGSGGSDIQMTQSPSSLSASVGDRVTITCSATSSITYMSWYQQKPGKAPKLLIYDTSNLASGVPSRFSGSGSGTDYTLTISSLQPEDFATYYCQQWSSYPLTFGGGTKVEIK

SEQ ID NO: 24 - protein, 482 aa, Artificial Sequence
  Description of Artificial Sequence: Synthetic polypeptide
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSGGSGGSGGSDIQMTQSPSSLSASVGDRVTITCSATSSITYMSWYQQKPGKAPKLLIYDTSNLASGVPSRFSGSGSGTDYTLTISSLQPEDFATYYCQQWSSYPLTFGGGTKVEIKGGSGGSGGSQVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSSGGSGGSGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 25 - DNA, 1476 bp, Artificial Sequence
  Description of Artificial Sequence: Synthetic polynucleotide
caagtgcagctccaagagtccggccccggcctcgtgaaacccagccagacactgtctctgacatgcaccgtgagcggcttttctctgaccagctacggagtgtattgggtgagacaaccccccggcaagggactggagtggctgggagtgatttgggccggcggcaccaccaactacaatagcgccctcatgtctagactcaccatctccaaggacaccagcaagaaccaagtgtccctcaagctgtccagcgtcacagctgccgacaccgccgtgtactattgtgctagaggccccccccatgccatgatgaagagaggctatgccatggattactggggccaaggcacactggtgaccgtcagctccggaggaagcggcggcagcggaggctccgaaattgtgctcacccagagccccggcacactgtctctgagccccggcgaaagggccacactgagctgcagagcctcccaatccgtgggcagcagctatctggcttggtatcagcagaaacccggccaagcccctagactgctgatctatggagccttttctagagctaccggcatccccgacagattctccggcagcggcagcggcacagactttacactgacaatttctagactggaaccagaggatttcgccgtctactactgccagcagtatggaagcagcccttggacctttggccaaggcaccaaggtggagatcaagggaggaagcggaggcagcggaggcagccaagtgcagctcgtggaaagcggaggaggcgtggtgcagcccggcagatccctcagactgagctgcgccgccagcggcttcaccttcagctcctataccatgcactgggtgaggcaagcccccggcaaaggactggagtgggtcaccttcatcagctacgacggcaacaacaagtactacgccgacagcgtgaagggaaggttcaccatctctagagacaactccaagaacaccctctacctccagatgaactctctgagggccgaagacaccgccatctactactgcgctagaaccggctggctgggaccctttgactactggggacaaggcacactggtcacagtgtcctccggaggaagcggaggctccggcggcagcgatatccagatgacccaatccccttcctctctgagcgcctccgtgggagatagggtcaccattacatgtagcgccacaagcagcatcacctacatgagctggtaccagcagaaacccggaaaggcccctaagctgctcatctacgacacctccaatctggccagcggcgtgccttctagatttagcggctccggaagcggcacagattacacactgacaatcagctctctgcagccagaggacttcgccacctactactgtcagcagtggagcagctaccctctgacctttggcggcggcaccaaggtggaaatcaaacaccaccatcaccaccatcaccaccatcac

SEQ ID NO: 26 - DNA, 1476 bp, Artificial Sequence
  Description of Artificial Sequence: Synthetic polynucleotide
caagtgcagctcgtggaatccggcggaggagtcgtgcagcccggcagaagcctcagactgagctgcgccgccagcggattcaccttcagcagctacaccatgcactgggtgaggcaagcccccggcaaaggactggagtgggtcacattcatctcctacgatggcaacaacaagtactacgccgacagcgtgaagggaaggtttaccatctctagagataactccaagaacaccctctacctccagatgaactctctgagagctgaggacacagccatctattactgcgctagaaccggatggctgggccctttcgactactggggacaaggcacactggtgacagtgtcctccggaggcagcggaggcagcggaggcagcgacatccagatgacccaatcccctagctctctgagcgccagcgtgggcgatagagtgaccattacatgctccgccaccagcagcatcacctacatgagctggtaccagcaaaagcccggcaaagcccccaagctgctgatctacgataccagcaatctggccagcggcgtgccttctagattttccggctccggaagcggcaccgattacacactgaccatttcctctctgcagccagaggatttcgccacctactattgccagcagtggtcctcctaccctctcacctttggcggcggaacaaaggtggagatcaaaggcggcagcggcggctccggaggcagccaagtccagctgcaagagtccggacccggactggtgaaaccttcccagacactgtctctgacatgcacagtgagcggattctccctcacaagctacggcgtgtactgggtgagacagcctcccggcaaaggactggagtggctgggcgtcatctgggctggaggcaccaccaattacaacagcgctctgatgtctagactgacaatctccaaggacaccagcaagaaccaagtgtctctgaagctgagctccgtgacagccgccgatacagccgtgtactattgtgccagaggccccccccacgctatgatgaagaggggctacgccatggactactggggccaaggcaccctcgtcacagtgagctccggaggcagcggaggaagcggcggcagcgagatcgtgctgacccagtcccccggcacactgtccctcagccccggcgaaagagccacactgagctgtagagcttcccagagcgtgggcagcagctatctggcttggtatcagcagaagcccggccaagcccccagactgctcatctacggagccttcagcagagccaccggcatccccgacagattcagcggcagcggcagcggcaccgattttaccctcaccatctccagactggagccagaggacttcgccgtgtattactgccaacagtacggcagcagcccttggacctttggacaaggcaccaaggtggaaatcaaacaccaccaccatcaccatcatcatcaccac

SEQ ID NO: 27 - protein, 216 aa, Artificial Sequence
  Description of Artificial Sequence: Synthetic polypeptide
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 28 - protein, 215 aa, Artificial Sequence
  Description of Artificial Sequence: Synthetic polypeptide
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 503 aa, Artificial Sequence
  Description of Artificial Sequence: Synthetic polypeptide
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKGGGGSGGGGSGGGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSKEAKEAKEAKEAKEAKEADIQMTQSPSSLSASVGDRVTITCSATSSITYMSWYQQKPGKAPKLLIYDTSNLASGVPSRFSGSGSGTDYTLTISSLQPEDFATYYCQQWSSYPLTFGGGTKVEIKGGGGSGGGGSGGGGSQVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSS

SEQ ID NO: 30 - protein, 503 aa, Artificial Sequence
  Description of Artificial Sequence: Synthetic polypeptide
DIQMTQSPSSLSASVGDRVTITCSATSSITYMSWYQQKPGKAPKLLIYDTSNLASGVPSRFSGSGSGTDYTLTISSLQPEDFATYYCQQWSSYPLTFGGGTKVEIKGGGGSGGGGSGGGGSQVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSSKEAKEAKEAKEAKEAKEAEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKGGGGSGGGGSGGGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 31 - protein, 478 aa, Artificial Sequence
  Description of Artificial Sequence: Synthetic polypeptide
QVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTGGGGSGGGGSGGGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHHHHHHHHHH

SEQ ID NO: 32 - protein, 213 aa, Artificial Sequence
  Description of Artificial Sequence: Synthetic polypeptide
DIQMTQSPSSLSASVGDRVTITCSATSSITYMSWYQQKPGKAPKLLIYDTSNLASGVPSRFSGSGSGTDYTLTISSLQPEDFATYYCQQWSSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 215 aa, Artificial Sequence; identical to SEQ ID NO: 28
  Description of Artificial Sequence: Synthetic polypeptide

SEQ ID NO: 34 - protein, 468 aa, Artificial Sequence
  Description of Artificial Sequence: Synthetic polypeptide
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTGGGGSGGGGSGGGGSQVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKT

SEQ ID NO: 35 - protein, 215 aa, Artificial Sequence; identical to SEQ ID NO: 28
  Description of Artificial Sequence: Synthetic polypeptide

SEQ ID NO: 36 - protein, 213 aa, Artificial Sequence; identical to SEQ ID NO: 32
  Description of Artificial Sequence: Synthetic polypeptide

SEQ ID NO: 37 - protein, 459 aa, Artificial Sequence
  Description of Artificial Sequence: Synthetic polypeptide
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKT

SEQ ID NO: 38 - protein, 224 aa, Artificial Sequence
  Description of Artificial Sequence: Synthetic polypeptide
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKT

SEQ ID NO: 39 - protein, 213 aa, Artificial Sequence; identical to SEQ ID NO: 32
  Description of Artificial Sequence: Synthetic polypeptide

SEQ ID NO: 40 - protein, 452 aa, Artificial Sequence
  Description of Artificial Sequence: Synthetic polypeptide
DIQMTQSPSSLSASVGDRVTITCSATSSITYMSWYQQKPGKAPKLLIYDTSNLASGVPSRFSGSGSGTDYTLTISSLQPEDFATYYCQQWSSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKT

SEQ ID NO: 41 - protein, 229 aa, Artificial Sequence
  Description of Artificial Sequence: Synthetic polypeptide
QVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKT

SEQ ID NO: 42 - protein, 215 aa, Artificial Sequence; identical to SEQ ID NO: 28
  Description of Artificial Sequence: Synthetic polypeptide

SEQ ID NO: 43 - protein, 273 aa, Artificial Sequence
  Description of Artificial Sequence: Synthetic polypeptide
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKGGGSGGGGQVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSSGGCGGGEVAALEKEVAALEKEVAALEKEVAALEK

SEQ ID NO: 44 - protein, 266 aa, Artificial Sequence
  Description of Artificial Sequence: Synthetic polypeptide
DIQMTQSPSSLSASVGDRVTITCSATSSITYMSWYQQKPGKAPKLLIYDTSNLASGVPSRFSGSGSGTDYTLTISSLQPEDFATYYCQQWSSYPLTFGGGTKVEIKGGGSGGGGQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSGGCGGGKVAALKEKVAALKEKVAALKEKVAALKE

SEQ ID NO: 45 - protein, 701 aa, Artificial Sequence
  Description of Artificial Sequence: Synthetic polypeptide
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPSPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFRLYSRLTVDKSRWQQGNVFSCSVMHEALHNHYTQKSLSLSPGGGGGSGGGGSDIQMTQSPSSLSASVGDRVTITCSATSSITYMSWYQQKPGKAPKLLIYDTSNLASGVPSRFSGSGSGTDYTLTISSLQPEDFATYYCQQWSSYPLTFGGGTKVEIKGGGGSGGGGSGGGGSQVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSS

SEQ ID NO: 46 - protein, 215 aa, Artificial Sequence; identical to SEQ ID NO: 28
  Description of Artificial Sequence: Synthetic polypeptide

SEQ ID NO: 47 - protein, 447 aa, Artificial Sequence
  Description of Artificial Sequence: Synthetic polypeptide
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 48 - protein, 481 aa, Artificial Sequence
  Description of Artificial Sequence: Synthetic polypeptide
DIQMTQSPSSLSASVGDRVTITCSATSSITYMSWYQQKPGKAPKLLIYDTSNLASGVPSRFSGSGSGTDYTLTISSLQPEDFATYYCQQWSSYPLTFGGGTKVEIKGGGGSGGGGSGGGGSQVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSSGGGGSEPKSQDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 49 - protein, 215 aa, Artificial Sequence; identical to SEQ ID NO: 28
  Description of Artificial Sequence: Synthetic polypeptide

SEQ ID NO: 50 - protein, 452 aa, Artificial Sequence
  Description of Artificial Sequence: Synthetic polypeptide
QVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 51 - protein, 478 aa, Artificial Sequence
  Description of Artificial Sequence: Synthetic polypeptide
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKGGGGSGGGGSGGGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSGGGGSEPKSQDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 52 - protein, 213 aa, Artificial Sequence; identical to SEQ ID NO: 32
  Description of Artificial Sequence: Synthetic polypeptide

SEQ ID NO: 53 - protein, 477 aa, Artificial Sequence
  Description of Artificial Sequence: Synthetic polypeptide
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKGGGGSGGGGSGGGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSGGGGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 54 - protein, 481 aa, Artificial Sequence; identical to SEQ ID NO: 48
  Description of Artificial Sequence: Synthetic polypeptide

SEQ ID NO: 55 - protein, 22 aa, Artificial Sequence
  Description of Artificial Sequence: Synthetic polypeptide
MDFQVQIFSFLLISASVILSRG

SEQ ID NO: 56-58 - skipped (3 entries)

SEQ ID NO: 59 - protein, 714 aa, Artificial Sequence
  Description of Artificial Sequence: Synthetic polypeptide
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSGGSGGSGGSDIQMTQSPSSLSASVGDRVTITCSATSSITYMSWYQQKPGKAPKLLIYDTSNLASGVPSRFSGSGSGTDYTLTISSLQPEDFATYYCQQWSSYPLTFGGGTKVEIKGGSGGSGGSQVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSSGGSGGSGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKGGSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - DNA, 2142 bp, Artificial Sequence
  Description of Artificial Sequence: Synthetic polynucleotide
caagtgcagctggtggagagcggtggcggcgtggtgcagcccggtagaagcctgagactgagctgcgccgcaagcggcttcaccttcagcagctacaccatgcactgggtgagacaagcacccggcaagggactggaatgggtcacgttcatcagctacgacggcaacaacaagtactacgccgacagcgtgaagggcagattcaccatcagccgagacaacagcaagaacaccctgtacctgcagatgaacagcctgagagccgaggacaccgccatctactattgcgctagaacgggatggctgggccccttcgattattgggggcaaggcacgttggtgaccgtctcaagcggcggaagcggaggcagcggaggatcggatatacagatgacacagtcgcctagctcactgagcgcaagcgtgggcgacagagtgaccatcacctgcagcgccacaagcagcatcacctacatgagctggtatcagcaaaaacccggcaaggcccccaagctgctgatctacgatacaagcaacctggcaagcggcgtgcctagcagattctctggcagcggcagcggcaccgactatactctcaccataagcagtctacagcctgaggatttcgccacctactattgtcagcagtggtcgagctaccccctgaccttcgggggcggcaccaaggtagagattaaaggtgggagcggcgggagtggtggcagccaagtccaactgcaagagtccggacccggtcttgtgaagccttctcagactcttagcttaacatgcaccgtgagcggcttcagccttacaagctacggcgtctactgggtacgtcaaccacctggtaagggtttagaatggctaggggtgatctgggccggcggcaccaccaactacaacagcgccctgatgagcagactgacgatctctaaggacactagcaaaaaccaagtgagcctcaagctgagcagcgtgaccgccgccgacaccgccgtatactactgtgcaagaggccccccccacgccatgatgaagagaggctacgccatggattactgggggcaaggcactctagtgaccgtgagtagtggcgggagcggtgggagcggcggttccgaaatcgtgctgacacagagccctggcactctgtctctgagtcccggcgagagagctaccctgagctgcagagcatctcagagcgtgggcagcagctacctggcctggtatcagcaaaagcccggccaagcccctagacttctgatctacggcgcattcagcagagccaccggcatccccgacagattcagtggatctggcagcggaacggacttcaccctaacaatcagccgtctggaacccgaagactttgcggtgtattactgtcaacagtacggtagcagcccctggaccttcggccaaggcacaaaggtggagatcaaaggcggttccggtggcgacaaaacacacacctgtcccccctgccccgcccctgagctgttaggtggtcctagcgtgttcctgttcccccccaagcccaaggacaccctgatgatcagcagaacccctgaggtgacctgcgtggtggtggacgtgagccacgaggaccctgaggtgaagttcaactggtacgtggacggcgtggaggtgcacaacgccaagaccaagcctagagaggagcagtacaacagcacctacagagtggtgagcgtgctgaccgtgctgcaccaagactggctgaacggcaaggagtacaagtgcaaggtgagcaacaaggccctgcccgcccccatcgagaagaccatcagcaaggccaaggggcagcctagagagccccaagtgtacaccctgccccctagcagagacgagctgaccaagaaccaagtgtcgctaacctgtttggtgaagggcttctaccctagcgacatcgccgtggagtgggagagcaacgggcagcctgagaacaactacaagaccaccccccccgtgctggacagcgacggcagcttcttcctgtacagcaagctgaccgtggacaagagcagatggcagcaaggcaacgtgttcagctgcagcgtgatgcacgaggccctgcacaaccactacacacagaagagcctgtcgctgagccccggaaaa

SEQ ID NO: 61 - protein, 714 aa, Artificial Sequence
  Description of Artificial Sequence: Synthetic polypeptide
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSGGSGGSGGSDIQMTQSPSSLSASVGDRVTITCSATSSITYMSWYQQKPGKAPKLLIYDTSNLASGVPSRFSGSGSGTDYTLTISSLQPEDFATYYCQQWSSYPLTFGGGTKVEIKGGSGGSGGSQVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSSGGSGGSGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - DNA, 2142 bp, Artificial Sequence
  Description of Artificial Sequence: Synthetic polynucleotide
caagtgcagctggtggagagcggtggcggcgtggtgcagcccggtagaagcctgagactgagctgcgccgcaagcggcttcaccttcagcagctacaccatgcactgggtgagacaagcacccggcaagggactggaatgggtcacgttcatcagctacgacggcaacaacaagtactacgccgacagcgtgaagggcagattcaccatcagccgagacaacagcaagaacaccctgtacctgcagatgaacagcctgagagccgaggacaccgccatctactattgcgctagaacgggatggctgggccccttcgattattgggggcaaggcacgttggtgaccgtctcaagcggcggaagcggaggcagcggaggatcggatatacagatgacacagtcgcctagctcactgagcgcaagcgtgggcgacagagtgaccatcacctgcagcgccacaagcagcatcacctacatgagctggtatcagcaaaaacccggcaaggcccccaagctgctgatctacgatacaagcaacctggcaagcggcgtgcctagcagattctctggcagcggcagcggcaccgactatactctcaccataagcagtctacagcctgaggatttcgccacctactattgtcagcagtggtcgagctaccccctgaccttcgggggcggcaccaaggtagagattaaaggtgggagcggcgggagtggtggcagccaagtccaactgcaagagtccggacccggtcttgtgaagccttctcagactcttagcttaacatgcaccgtgagcggcttcagccttacaagctacggcgtctactgggtacgtcaaccacctggtaagggtttagaatggctaggggtgatctgggccggcggcaccaccaactacaacagcgccctgatgagcagactgacgatctctaaggacactagcaaaaaccaagtgagcctcaagctgagcagcgtgaccgccgccgacaccgccgtatactactgtgcaagaggccccccccacgccatgatgaagagaggctacgccatggattactgggggcaaggcactctagtgaccgtgagtagtggcgggagcggtgggagcggcggttccgaaatcgtgctgacacagagccctggcactctgtctctgagtcccggcgagagagctaccctgagctgcagagcatctcagagcgtgggcagcagctacctggcctggtatcagcaaaagcccggccaagcccctagacttctgatctacggcgcattcagcagagccaccggcatccccgacagattcagtggatctggcagcggaacggacttcaccctaacaatcagccgtctggaacccgaagactttgcggtgtattactgtcaacagtacggtagcagcccctggaccttcggccaaggcacaaaggtggagatcaaaggcggttccggtggcgacaaaacacacacctgtcccccctgccccgcccctgaggctgccggtggtcctagcgtgttcctgttcccccccaagcccaaggacaccctgatgatcagcagaacccctgaggtgacctgcgtggtggtggacgtgagccacgaggaccctgaggtgaagttcaactggtacgtggacggcgtggaggtgcacaacgccaagaccaagcctagagaggagcagtacaacagcacctacagagtggtgagcgtgctgaccgtgctgcaccaagactggctgaacggcaaggagtacaagtgcaaggtgagcaacaaggccctgggcgcccccatcgagaagaccatcagcaaggccaaggggcagcctagagagccccaagtgtacaccctgccccctagcagagacgagctgaccaagaaccaagtgtcgctaacctgtttggtgaagggcttctaccctagcgacatcgccgtggagtgggagagcaacgggcagcctgagaacaactacaagaccaccccccccgtgctggacagcgacggcagcttcttcctgtacagcaagctgaccgtggacaagagcagatggcagcaaggcaacgtgttcagctgcagcgtgatgcacgaggccctgcacaaccactacacacagaagagcctgtcgctgagccccggaaaa

SEQ ID NO: 63 - protein, 1081 aa, Artificial Sequence
  Description of Artificial Sequence: Synthetic polypeptide
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSGGSGGSGGSDIQMTQSPSSLSASVGDRVTITCSATSSITYMSWYQQKPGKAPKLLIYDTSNLASGVPSRFSGSGSGTDYTLTISSLQPEDFATYYCQQWSSYPLTFGGGTKVEIKGGSGGSGGSQVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSSGGSGGSGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKGGSGGEAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAHHHHHHHHHH

SEQ ID NO: 64 - DNA, 3243 bp, Artificial Sequence
  Description of Artificial Sequence: Synthetic polynucleotide
caagtgcaattggtagagtccggcggcggcgttgtgcaacccggcagaagccttagactgagctgcgccgcaagcggcttcaccttcagcagctacaccatgcactgggtgagacaagcccccggcaagggcctggaatgggtgaccttcatcagctacgacggcaacaacaagtactacgctgatagcgtgaagggcagattcactatcagccgtgacaacagcaagaacaccctgtacctgcagatgaacagcctgagagccgaggacaccgccatctactactgcgcaagaaccggctggctgggccccttcgactactggggccaagggacattagttactgttagcagcggcgggagcgggggcagcggaggatcggatatacagatgacacagagccctagcagcctgagcgcaagcgtgggcgacagagtgaccatcacctgcagcgccacaagcagcatcacctacatgagctggtatcagcaaaaacccggcaaggcccccaagctgctgatctacgacacaagcaacctggcaagcggcgtgcctagcagattctctggcagcggcagcggcacagattatactctcacaatcagcagtctgcagcctgaggatttcgccacatactactgccaacagtggagcagctaccccctgaccttcggcggcggcactaaagtagagattaaaggcggcagcggtggaagtggcgggagccaagtgcaactgcaagagagcggtcccggcctggtgaagccttctcagaccttgagccttacctgcaccgtgagcggcttcagcctgacaagctacggcgtgtactgggtgcggcaaccccccggcaaaggcttggaatggctgggggtgatctgggccggcggcaccaccaactacaacagcgccctgatgagcagactgaccattagcaaggacacaagcaagaaccaagtgagcctgaagctatctagcgtgaccgccgccgacaccgccgtctattactgtgcaagaggccccccccacgccatgatgaagagaggctacgccatggattattggggccaaggcaccctggttaccgtctcttccgggggtagcggcggcagcggaggttccgagatcgtgttaactcagagccctggcaccctgtcgctgagccctggtgaaagggctacgctaagctgcagagcatctcagagcgtgggcagcagctacctggcctggtatcagcagaaacccggccaagcccctagactgctgatctatggtgccttcagcagagccaccggcatccccgacagattcagcggttccggtagtggcacagacttcactctgaccatcagccggctagaacctgaggattttgccgtttactactgtcagcagtatggcagcagcccctggaccttcggccaaggcacgaaggtggagataaaaggtggaagcggcggggaagcccacaagagcgagatcgcccacagatacaacgacctgggcgagcagcacttcaagggcctggtgctgatcgccttctctcagtacctgcagaagtgcagctacgacgagcacgccaagctggtgcaagaggtgaccgacttcgccaagacctgcgtggccgacgagagcgccgccaactgcgacaagagcctgcacaccctgttcggcgacaagctgtgcgccatccccaacctgagagagaactacggcgagctggccgactgctgcaccaagcaagagccggagagaaatgagtgcttcctgcagcacaaggacgacaaccctagcctgccccccttcgagagacctgaggccgaggccatgtgcactagcttcaaggagaaccccaccaccttcatgggccactacctgcacgaggtggctagacgtcatccatatttctacgcccctgagctgctgtactatgcagagcagtacaacgagatcctgacccaatgttgtgccgaggcggacaaagagagctgcctgacccccaagctggacggcgtgaaggagaaggccctggtgagcagcgtcagacagagaatgaagtgcagcagcatgcagaagttcggggagagagctttcaaggcctgggccgtggctagactgtctcagaccttccccaacgccgacttcgccgagatcaccaagctggccaccgacctgaccaaggtgaacaaggagtgctgccacggcgacctgctggagtgcgccgacgacagagccgagctggccaagtacatgtgcgagaaccaagccactatctcatccaaattacagacctgctgcgacaagcccctgctgaagaaagctcattgcctcagcgaggtggagcatgacaccatgcccgccgatctgcccgccatcgccgccgacttcgtggaggaccaagaggtgtgcaagaactacgccgaggcgaaggatgtgttcctgggcaccttcctgtacgagtacagccgaaggcaccccgactacagcgtgagcctgctgctgagactggccaagaagtacgaggccactttggagaaatgctgcgctgaggcaaacccccctgcctgctacggcaccgtgctggccgagtttcagcccctggtagaggagccgaagaacctggtgaagaccaactgcgacctgtacgagaagctgggcgagtacggctttcagaacgccatcctggtgagatacacacagaaggccccccaagtgagcacccccaccttggtggaagccgcaagaaacctgggcagagtgggcaccaaatgttgcacgctgccggaggaccaaagactgccctgcgtggaggactacctgagcgccatcctgaacagagtgtgcctgctgcacgagaagacccccgtgagcgagcacgtgaccaagtgctgcagtggcagcctggtggagagaagaccctgcttcagcgccctgaccgtggacgagacctacgtgcccaaggagttcaaggccgagaccttcaccttccacagcgacatctgtactctccctgagaaggagaagcagatcaagaagcagaccgccctggccgagctggtgaagcacaagcccaaggccaccgccgagcagctgaagaccgtgatggacgacttcgctcagttcctggacacctgctgcaaggccgccgacaaggacacctgcttcagcaccgagggccccaacctggtgacaagatgcaaggacgccctggcccaccaccaccaccaccaccaccaccaccac

SEQ ID NO: 65 - protein, 449 aa, Artificial Sequence
  Description of Artificial Sequence: Synthetic polypeptide
DVQLQESGPGLVKPSQSLSLTCSVTGYSITSGYVWNWIRQFPGNKLEWMGYISHDGNTNYNPSLKNRISITRDTSKNQFFLKLNSVTTEDSATYYCTRNYGYGGTMDYWGQGTAVSVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 66 - DNA, 1347 bp, Artificial Sequence
  Description of Artificial Sequence: Synthetic polynucleotide
gacgtgcagctgcaagagagcggccccggcctggtgaagccgagccaatctctgagcctgacctgcagtgtgacgggatactcaatcacctctggttacgtgtggaactggatcagacagttccccggcaacaagctggagtggatgggctacatcagccacgacggcaacaccaactacaaccctagcctgaagaacagaatcagcataacaagagacacaagcaagaatcagttcttcctgaagctgaacagcgtgaccaccgaggacagcgccacctactactgcacaagaaactacggctacggcggcaccatggactactggggccaaggcaccgccgtgagcgtaagcagcgccaagacaacagcccctagcgtgtacccgctggcccccgtttgcggcgacaccaccggtagcagcgtgaccctggggtgcctggtgaagggctacttccctgagcccgtgaccctgacttggaatagcggcagcctgagcagcggcgtgcacaccttccccgccgtgctgcagagcgacctgtacaccctgagcagcagcgtgacagtgacgagcagcacctggcctagccaaagtatcacctgcaacgtggcccaccccgcaagcagcaccaaggtggacaagaagatcgaaccaagaggccccactattaaaccttgccccccctgcaagtgtcccgcacccaatgcagcaggcggccctagcgtgttcatcttcccccccaagatcaaggacgtgctgatgatcagcctgagccccatcgtgacctgcgtggtggtggacgtgagcgaggacgaccccgacgtgcagatcagctggttcgtgaacaacgtggaggtgcacaccgctcagacacagacccacagagaggactacaacagcaccctgagagtggtgagcgccctgcccattcagcaccaagactggatgagcggcaaggagttcaagtgcaaggtgaacaacaaggacctgggcgcccccatcgagagaaccatcagcaagcccaagggcagcgtgagagccccccaagtgtacgtgctgcccccccctgaggaggagatgaccaagaagcaagtgaccctaacctgtatggtgaccgacttcatgcctgaggacatctacgtggagtggaccaacaacggcaagaccgagctgaactacaagaacaccgagcccgtgctggacagcgacggcagctacttcatgtacagcaagctgagagtggagaagaagaactgggtggagagaaacagctacagctgcagcgtggtgcacgagggcctgcacaaccaccacaccaccaagagcttcagcagaacccccggcaag

SEQ ID NO: 67 - protein, 214 aa, Artificial Sequence
  Description of Artificial Sequence: Synthetic polypeptide
DIVMTQSQKFMSTSVGDRVSVTCKASQNVGTYVAWYEQKLGQSPKALIFSASYRYTGVPDRFTGSGSGTDFTLTISNVQSEDLAEYFCQQYDSYPLTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 68 - DNA, 642 bp, Artificial Sequence
  Description of Artificial Sequence: Synthetic polynucleotide
gacatcgtgatgacacagtctcagaagttcatgagcacaagcgtgggcgacagagtgagcgtgacctgcaaggcatctcagaacgtgggcacctacgtggcctggtacgagcagaagctggggcagagccccaaggccctgatcttcagcgcaagctacagatataccggcgtgcccgacagattcaccggcagcggcagcggcaccgacttcaccctgaccatcagcaacgtgcagagcgaggacctggccgagtacttctgtcagcagtacgacagctaccccctgaccttcggcggcggcaccaagctcgagatcaagcgcgcagatgctgctcctaccgtgagcatcttcccgccgtccagcgaacaactcactagcggaggcgcgtcagtggtctgcttccttaacaatttctaccctaaggacatcaacgtcaagtggaagattgacggatcggaacgccagaacggagtgctgaactcatggactgatcaggattccaaagactcgacttactccatgtccagcaccctgaccctgaccaaagacgagtacgaaaggcacaactcgtacacgtgcgaagccacccacaagacttccacctcgcccatcgtgaagtccttcaatcgcaatgagtgc

SEQ ID NO: 69 - protein, 448 aa, Artificial Sequence
  Description of Artificial Sequence: Synthetic polypeptide
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - DNA, 1344 bp, Artificial Sequence
  Description of Artificial Sequence: Synthetic polynucleotide
caagtgcagctagttgagtctggaggcggcgtggtgcagcccggcagaagcctgagactgagctgcgccgcaagcggcttcaccttcagcagctacaccatgcactgggtgagacaagcccccggcaagggcctggagtgggtgaccttcatcagctacgacggcaacaacaagtactacgccgacagcgtgaagggcagattcaccatcagcagagacaacagcaagaacaccctgtacctgcagatgaacagcctgagagccgaggacaccgccatctactactgcgctagaaccggctggctgggccccttcgactactggggccaaggcaccctggtaacggtaagcagcgctagtaccaaagggcctagcgtgtttcctttagcccctagcagcaaaagcacaagcggaggcaccgccgccctgggctgtctggtgaaggattactttcctgaacccgtgactgtatcatggaatagcggggccctgactagcggagtgcataccttccccgccgtgctgcagagcagcggcctgtacagcctgagcagcgtggtgaccgtgcctagcagcagcctgggcacacagacctacatctgcaacgtgaaccacaagcctagcaacaccaaggtggacaagaaggtggagcccaagagctgtgacaaaacgcacacctgtccgccgtgccccgcccctgaggctgccggcggccctagcgtgttcctgtttccccccaagcccaaggacaccctgatgatcagcagaacccctgaggtgacctgcgtggtggtggacgtgagccacgaggaccctgaggtgaagttcaactggtacgtggacggcgtggaggtgcacaacgccaagaccaagcctagagaggagcagtacaacagcacctacagagtggtgagcgtgctgaccgtgctgcaccaagactggctgaacggcaaggagtacaagtgcaaggtgagcaacaaggccctgggcgcccccatcgagaagaccatcagcaaggccaaggggcagcctagagagccccaagtgtacaccctgccccctagcagagacgagctgaccaagaaccaagtgagcctgacctgcctagtgaagggtttctaccctagcgacatcgccgtggagtgggagagcaacgggcagcctgagaacaactacaagaccaccccccccgtgctggacagcgacggcagcttcttcctgtacagcaagctgaccgtggacaagagcagatggcagcaaggcaacgtgttcagctgcagcgtgatgcacgaggccctgcacaaccactacacacagaagagcctgagcctgagccccggcaag

SEQ ID NO: 71 - protein, 215 aa, Artificial Sequence; identical to SEQ ID NO: 28
  Description of Artificial Sequence: Synthetic polypeptide

SEQ ID NO: 72 - DNA, 651 bp, Artificial Sequence
  Description of Artificial Sequence: Synthetic polynucleotide
gagatcgtgctgacacagagccccggcacactgtctctgagccccggcgagagagccacactgagctgcagagccagccagagcgtgggcagcagctatctggcttggtaccagcagaagcccggccaagcccctagactgctgatttacggagcctttagcagagccaccggcatccccgacagattcagcggaagcggcagcggcaccgatttcacactgacaatctctagactggagccagaggacttcgccgtgtactactgccagcagtacggcagcagcccttggacctttggccaaggcaccaaggtggagatcaagatcaagagaactgtggccgcgccgtcagtgtttatcttccctccatcggatgaacagcttaagtccggcacggcgtctgtggtctgcctgctcaataacttttaccctagggaagctaaagtccaatggaaagtggataacgccctgcagtcaggaaacagccaggaatcggttaccgaacaggacagcaaggacagcacttactccttgtcgtcgactcttactctgagcaaggccgattacgagaagcacaaggtctacgcctgcgaggtcacccatcagggactctcgtccccggtgaccaaatccttcaatagaggcgaatgc

SEQ ID NO: 73 - protein, 4 aa, Artificial Sequence
  Description of Artificial Sequence: Synthetic polypeptide
YVKM

SEQ ID NO: 74 - protein, 60 aa, Artificial Sequence
  Description of Artificial Sequence: Synthetic polypeptide
  SITE (1)..(60): This sequence may encompass 1-20 ′Gly Gly Ser′ repeating units
GGSGGSGGSGGSGGSGGSGGSGGSGGSGGSGGSGGSGGSGGSGGSGGSGGSGGSGGSGGS

SEQ ID NO: 75 - protein, 9 aa, Artificial Sequence
  Description of Artificial Sequence: Synthetic polypeptide
GGSGGSGGS

SEQ ID NO: 76 - protein, 8 aa, Artificial Sequence
  Description of Artificial Sequence: Synthetic polypeptide
EPKSCDKT

SEQ ID NO: 77 - protein, 34 aa, Artificial Sequence
  Description of Artificial Sequence: Synthetic polypeptide
GGCGGGEVAALEKEVAALEKEVAALEKEVAALEK

SEQ ID NO: 78 - protein, 6 aa, Artificial Sequence
  Description of Artificial Sequence: Synthetic polypeptide
HHHHHH

SEQ ID NO: 79 - protein, 492 aa, Artificial Sequence
  Description of Artificial Sequence: Synthetic polypeptide
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSGGSGGSGGSDIQMTQSPSSLSASVGDRVTITCSATSSITYMSWYQQKPGKAPKLLIYDTSNLASGVPSRFSGSGSGTDYTLTISSLQPEDFATYYCQQWSSYPLTFGGGTKVEIKGGSGGSGGSQVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSSGGSGGSGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKHHHHHHHHHH

SEQ ID NO: 80 - protein, 492 aa, Artificial Sequence
  Description of Artificial Sequence: Synthetic polypeptide
QVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSSGGSGGSGGSEIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKGGSGGSGGSQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSGGSGGSGGSDIQMTQSPSSLSASVGDRVTITCSATSSITYMSWYQQKPGKAPKLLIYDTSNLASGVPSRFSGSGSGTDYTLTISSLQPEDFATYYCQQWSSYPLTFGGGTKVEIKHHHHHHHHHH

SEQ ID NO: 81 - protein, 226 aa, Artificial Sequence
  Description of Artificial Sequence: Synthetic polypeptide
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSGGGGS

SEQ ID NO: 82 - protein, 217 aa, Artificial Sequence
  Description of Artificial Sequence: Synthetic polypeptide
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - protein, 234 aa, Artificial Sequence
  Description of Artificial Sequence: Synthetic polypeptide
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHHHHHHHHHH

SEQ ID NO: 84 - protein, 231 aa, Artificial Sequence
  Description of Artificial Sequence: Synthetic polypeptide
QVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSGGGGS

SEQ ID NO: 85 - skipped

SEQ ID NO: 86 - protein, 239 aa, Artificial Sequence
  Description of Artificial Sequence: Synthetic polypeptide
QVQLQESGPGLVKPSQTLSLTCTVSGFSLTSYGVYWVRQPPGKGLEWLGVIWAGGTTNYNSALMSRLTISKDTSKNQVSLKLSSVTAADTAVYYCARGPPHAMMKRGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHHHHHHHHHH

SEQ ID NO: 87 - skipped

SEQ ID NO: 88 - protein, 213 aa, Artificial Sequence; identical to SEQ ID NO: 22
  Description of Artificial Sequence: Synthetic polypeptide

SEQ ID NO: 89-90 - skipped (2 entries)

SEQ ID NO: 91 - protein, 66 aa, Artificial Sequence
  Description of Artificial Sequence: Synthetic polypeptide
SGALTSGVHTFPAVLQSSGLYSLSSVVTVPSSSLGTQTYICNVNHKPSNTKVDKKVGGGGSGGGGS

SEQ ID NO: 92 - protein, 74 aa, Artificial Sequence
  Description of Artificial Sequence: Synthetic polypeptide
SGALTSGVHTFPAVLQSSGLYSLSSVVTVPSSSLGTQTYICNVNHKPSNTKVDKKVEPKSCDKTHHHHHHHHHH

SEQ ID NO: 93 - protein, 70 aa, Artificial Sequence
  Description of Artificial Sequence: Synthetic polypeptide
TSGVHTFPAVLQSSGLYSLSSVVTVPSSSLGTQTYICNVNHKPSNTKVDKKVEPKSCDKTHHHHHHHHHH

SEQ ID NO: 94 - protein, 10 aa, Artificial Sequence
  Description of Artificial Sequence: Synthetic polypeptide
HHHHHHHHHH

SEQ ID NO: 95 - protein, 62 aa, Artificial Sequence
  Description of Artificial Sequence: Synthetic polypeptide
TSGVHTFPAVLQSSGLYSLSSVVTVPSSSLGTQTYICNVNHKPSNTKVDKKVGGGGSGGGGS

SEQ ID NO: 96 - skipped

SEQ ID NO: 97 - protein, 220 aa, Artificial Sequence
  Description of Artificial Sequence: Synthetic polypeptide
  MOD_RES (1)..(3): Any amino acid
XXXLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFI

SEQ ID NO: 98 - protein, 15 aa, Artificial Sequence
  Description of Artificial Sequence: Synthetic polypeptide
GGGGSGGGGSGGGGS

SEQ ID NO: 99 - protein, 34 aa, Artificial Sequence; identical to SEQ ID NO: 77
  Description of Artificial Sequence: Synthetic polypeptide

SEQ ID NO: 100 - protein, 34 aa, Artificial Sequence
  Description of Artificial Sequence: Synthetic polypeptide
GGCGGGKVAALKEKVAALKEKVAALKEKVAALKE

SEQ ID NO: 101 - protein, 8 aa, Artificial Sequence
  Description of Artificial Sequence: Synthetic polypeptide
GGGSGGGG

SEQ ID NO: 102 - protein, 18 aa, Artificial Sequence
  Description of Artificial Sequence: Synthetic polypeptide
KEAKEAKEAKEAKEAKEA

(19) United States
(12) Patent Application Publication
(10) Pub. No.: US 2025/0282870 A1
(43) Pub. Date: Sep. 11, 2025

(54) CTLA4-BINDING PROTEINS AND METHODS OF TREATING CANCER

(71) Applicant: BioEntre LLC, Brookline, MA (US)

(72) Inventors: Gordon Gokleun Wong, Brookline, MA (US); Vincent Ling, Walpole, MA (US)

(21) Appl. No.: 18/273,331

(22) PCT Filed: Jan. 20, 2022
(86) PCT No.: PCT/US22/13120

§ 371 Date: Jul. 20, 2023

Related U.S. Application Data
(60) Provisional application No. 63/139,510, filed on Jan. 20, 2021.

(51) Int. Cl. C07K 16/28; A61K 39/00; A61P 35/00

(52) U.S. Cl. CPC C07K 16/2818; A61P 35/00; A61K 2039/505; C07K 2317/34; C07K 2317/52; C07K 2317/56; C07K 2317/626; C07K 2317/71; C07K 2317/76; C07K 2319/21

(57) ABSTRACT

The present disclosure provides engineered antigen-binding proteins that bind to cytotoxic T-lymphocyte-associated antigen-4 (CTLA4). Nucleic acids, vectors, host cells, and conjugates are also provided herein. Further provided are kits and pharmaceutical compositions comprising said entities as well as methods of making the engineered antigen-binding proteins and methods of treating a subject in need thereof.

### RELATED APPLICATION

This application is the U.S. National Stage of International Patent Application No. PCT/US22/13120, filed Jan. 20, 2022, which claims the benefit of priority to U.S. Provisional Patent Application No. 63/139,510, filed on Jan. 20, 2021, the contents of both applications are fully incorporated by reference herein.

### INCORPORATION BY REFERENCE OF SEQUENCE LISTING

The instant application contains a Sequence Listing which has been submitted electronically in ASCII format and is hereby incorporated by reference in its entirety. Said ASCII copy, created on Feb. 5, 2024, is named BES-00125 SL.txt and is 212,712 bytes in size.

### BACKGROUND

The striking clinical success of cancer immunotherapy with checkpoint blockade suggests it is likely to form the foundation of curative therapy for many malignancies (Reck et al. (2016) N. Engl. J. Med. 375:1823-1833; Hodi et al. (2010) N. Engl. J. Med. 363:711-723). The cytotoxic T-lymphocyte-associated antigen-4 (CTLA4) is an immune checkpoint that regulates T-cell proliferation at the early stage of naive T-cell activation, principally in the lymph nodes, thus providing a negative signal to T cells. Blockade of CTLA4 binding to its cognate ligand(s) induces an antitumor immune response by promoting the activation and proliferation of tumor-specific T cells.

Ipilimumab (Yervoy), a human monoclonal antibody that binds to human CTLA4 and blocks its interaction with ligands, demonstrated clinical efficacy in patients with melanoma, renal cell carcinoma, prostate cancer, urothelial carcinoma, and ovarian cancer. In 2011, ipilimumab was approved by the U.S. Food and Drug Administration (FDA) for the treatment of melanoma, and its 2020 sale is estimated at $1.7 billion. However, ipilimumab has only a 22% long-term success rate in melanoma eradication, albeit effectively a cure. Additionally, ipilimumab is highly toxic with many side effects characteristic of autoimmune disease and TREG (Regulatory T cells) depletion, thereby limiting its broad use. Accordingly, a great need exists in the art for additional immunotherapeutic strategies with better efficacy and safety profiles.

### SUMMARY

The present invention is based, at least in part, on the discovery that certain CTLA4-binding proteins show unexpected T cell activation properties. These CTLA4-binding proteins include those that bind multiple epitopes on CTLA4, e.g., Epitope 1 (¹³⁴MYPPPY¹³⁹ (SEQ ID NO: 1)) and Epitope 2 (⁶⁵SICT⁶⁸ (SEQ ID NO: 2)), as well as the entities that are devoid of the Fc region of the antibody (e.g., F(ab′)2 or diabodies). Surprisingly, the biparatopic entities presented herein showed synergistic effects on blocking CTLA4 ligand binding and T cell activation over the combination of monoparatopic counterparts (e.g., ipilimumab). In addition, the activity of the CTLA4-binding proteins devoid of the Fc domain surpassed the activity of ipilimumab or other CTLA4-binding antibodies with the Fc domain. Such findings were surprising and unexpected as it is widely accepted that the interaction between the Fc region of a CTLA4 antibody with the Fc receptors is important for CTLA4-blocking activity (Bulliard et al. (2013) J of Exp Medicine 9:1685-1693; Waight et al. (2018) Cancer Cell 33:1033-1047; Ingram et al. (2018) Proc Natl Acad Sci USA 115:3912-3917; Vargas et al. (2018) Cancer Cell 33:1-15). Thus, the engineered antigen-binding proteins and pharmaceutical compositions comprising same promise clinical efficacy that surpasses that of ipilimumab.

In certain aspects, provided herein is an engineered antigen-binding protein that specifically binds to Epitope 1 defined by the residues ¹³⁴MYPPPY¹³⁹ (SEQ ID NO: 1) and Epitope 2 defined by the residues ⁶⁵SICT⁶⁸ (SEQ ID NO: 2) of CTLA4.

In certain aspects, provided herein is an engineered antigen-binding protein that specifically binds to CTLA4 and lacks the CH2 domain and/or a CH3 region of the constant region of an antibody. In some embodiments, the engineered antigen-binding protein is a diabody.

In certain aspects, provided herein is an isolated nucleic acid molecule that encodes an engineered antigen-binding protein of the present disclosure. Also provided herein is a vector comprising such nucleic acid. Further provided herein is a host cell which comprises the isolated nucleic acid, comprises a vector, or expresses an engineered antigen-binding protein of the present disclosure.

In certain aspects, provided herein is a pharmaceutical composition of an engineered antigen-binding protein of the present disclosure, the isolated nucleic acid, the vector, or the host cell. Further provided herein is a kit comprising at least one engineered antigen-binding protein of the present disclosure.

In certain aspects, provided herein is a method of producing at least one engineered antigen-binding protein of the present disclosure, wherein the method comprises the steps of: (i) culturing a host cell comprising a nucleic acid comprising a sequence encoding at least one engineered antigen-binding protein of the present disclosure under conditions suitable to allow expression of said engineered antigen-binding protein; and (ii) recovering the expressed engineered antigen-binding protein.

In certain aspects, provided herein is a method of preventing or treating a subject afflicted with cancer, the method comprising administering to the subject at least one engineered antigen-binding protein of the present disclosure or a pharmaceutical composition comprising same.

In certain aspects, provided herein is a method of reducing proliferation of a cancer cell in a subject in need thereof, the method comprising administering to the subject at least one engineered antigen-binding protein of the present disclosure or a pharmaceutical composition comprising same.

Numerous embodiments are further provided that can be applied to any aspect of the present invention and/or combined with any other embodiment described herein. For example, in some embodiments, the at least one engineered antigen-binding protein or the pharmaceutical composition (a) reduces the number of proliferating cancer cells in the cancer; (b) reduces the volume or size of a tumor of the cancer; (c) increases the immune response against the cancer; and/or (d) activates the T cell.

In some embodiments, the engineered antigen-binding protein comprises an Fc domain that is heterologous to the antigen-binding domain. In some embodiments, the heterologous Fc domain extends the half-life of the antigen-binding protein. In some embodiments, the Fc domain is an IgG1 Fc. In still other embodiments, the IgG1 Fc comprises a LALAPG mutation (Lo et al., (2017) J. Biological Chem., 292:3900-08).

In some embodiments, the method further comprises administering to the subject an additional cancer therapy. In some embodiments, the additional cancer therapy is selected from the group consisting of immunotherapy, checkpoint blockade, cancer vaccines, chimeric antigen receptors, chemotherapy, radiation, target therapy, and surgery. In one embodiment, the additional cancer therapy is nivolumab.

In some embodiments, the cancer is selected from pancreatic cancer, lung cancer, non-small cell lung cancer (NSCLC), malignant pleural mesothelioma, small cell lung cancer (SCLC), renal cell carcinoma (RCC), breast cancer, liver cancer, hepatocellular carcinoma, kidney cancer, skin cancer, melanoma, thyroid cancer, gall bladder cancer, head-and-neck (squamous) cancer, stomach (gastric) cancer, head and neck cancer, bladder cancer, urothelial carcinoma, Merkel cell cancer, colon cancer, colorectal cancer, intestinal cancer, ovarian cancer, cervical cancer, testicular cancer, esophageal cancer, buccal cancer, brain cancer, blood cancers, lymphomas (B and T cell lymphomas), mesothelioma, cutaneous squamous cell cancer, Hodgkin's lymphoma, B-cell lymphoma, and a malignant or metastatic form thereof.

In some embodiments, the cancer is selected from melanoma (e.g., unresectable or metastatic melanoma), renal cell carcinoma (RCC), colorectal cancer, hepatocellular carcinoma, non-small cell lung cancer (NSCLC), malignant pleural mesothelioma, small cell lung cancer (SCLC), breast cancer, head and neck cancer, bladder cancer, urothelial carcinoma, Merkel cell cancer, cervical cancer, hepatocellular carcinoma, gastric cancer, cutaneous squamous cell cancer, Hodgkin's lymphoma, and B-cell lymphoma.

In certain aspects, provided herein is a method of increasing an immune response in a subject, the method comprising administering to the subject at least one engineered antigen-binding protein of the present disclosure or a pharmaceutical composition comprising same.

In certain aspects, provided herein is a method of activating a T cell, the method comprising contacting the T cells with at least one engineered antigen-binding protein of the present disclosure or a pharmaceutical composition comprising same.

In certain aspects, provided herein is a method of preventing or treating a disease or a condition characterized by aberrant expression or activity of a CTLA4 protein in a subject in need thereof, the method comprising administering to the subject at least one engineered antigen-binding protein of the present disclosure or a pharmaceutical composition comprising same. In some embodiments, the disease or condition is a cancer, autoimmune disease, infection, or inflammatory disease.

### BRIEF DESCRIPTION OF THE DRAWINGS

FIG. 1 shows the CTLA4 blockade assay. The bioassay consists of two genetically engineered cell lines, CTLA4 Effector Cells (Jurkat) and aAPC/Raji Cells. When co-cultured, the CTLA4/CD80 and CD86 interaction inhibits the CD28 pathway activated luminescence (left panel). The addition of anti-CTLA4 antibody blocks the CTLA4/CD80 and CD86 interaction, thereby re-establishing the CD28 pathway activated luminescence, which can be detected in a dose-dependent manner by addition of a luminescent agent (Glo) and quantitation with a luminometer (middle panel). When co-cultured with non-CTLA4-expressing Effector Cells (right panel), activation also induces luminescence by activation of the CD28 pathway but in a manner independent of anti-CTLA4 antibody.

FIG. 2A shows the CTLA4 blockade dose response to ipilimumab (“Ipi” or “IPI”), L3D10, or a combination of IPI and L3D10.

FIG. 2B shows the CTLA4 blockade dose response to ipilimumab (“Ipi” or “IPI”) or L3D10 with the IL-2 level as an assay output.

FIG. 3 shows that anti-CTLA4 antibodies have the CTLA4 blocking activity that depends on the Fc receptor, CD32. The addition of an anti-CD32 antibody reduces the CTLA4 blockade mediated by various anti-CTLA4 antibodies. The antibodies tested herein include Ipi (ipilimumab), aCD32 (anti-CD32 antibody), L3D10 (BioLegend anti-CTLA4 antibody), and 26 HuIgG1.

FIG. 4 shows that anti-CTLA4 antibodies have the CTLA4 blocking activity that depends on the Fc receptor, CD32. The addition of an anti-CD32 antibody reduces the CTLA4 blockade mediated by various anti-CTLA4 antibodies, but not the antibody containing the LALA mutation that abrogates the interaction between the antibody's Fc region and CD32.

FIG. 5 shows that the 26 Fab′ or 26 F(ab′)2 has an increased CTLA4 blocking activity as compared with the 26 HuIgG1 full-length antibody.

FIG. 6 shows that the increased CTLA4-blocking activity of 26 F(ab′)2 is independent of CD32.

FIG. 7 shows the CTLA4-blocking activity of ipilimumab, Ipi F(ab′)2, 121 HuIgG1, and 121 F(ab′)2.

FIG. 8 shows the CTLA4-blocking activity of ipilimumab, Ipi scFv, 121 HuIgG1, and 121 scFv.

FIGS. 9A and 9B show a schematic diagram of various antigen-binding proteins of the present disclosure. FIG. 9A shows the domain structures of various diabodies. The diagram is adapted from Kipriyanov et al. (1999) J Mol Biol 293:41-56, Volkel et al. (2001) Protein Engineering 14:815-823, Gall et al. (2004) Protein Engineering, Design & Selection 17:357-366, and Reusch et al. (2014) mAbs 6:727-738. FIG. 9B shows a schematic diagram of different types of antigen-binding proteins. FIG. 9A discloses “His6” as SEQ ID NO: 78.

FIGS. 10A-FIG. 10I show the domain structure and sequences of various diabodies. FIG. 10A shows the domain structure of exemplary anti-CTLA4 diabodies (BioE2051 and BioE2052). The diabodies comprise the VL and VH domains of ipilimumab and 121 antibody, and target two independent epitopes on CTLA4. FIG. 10A discloses “(G2S) 3” as SEQ ID NO: 75 and “His6” as SEQ ID NO: 78. FIG. 10B shows the diagram and sequence of BioE2052 (SEQ ID NO: 79). FIG. 10B discloses “His10” as SEQ ID NO: 92. FIG. 10C shows the diagram and sequence of BioE2051 (SEQ ID NO: 80). FIG. 10C discloses “His10” as SEQ ID NO: 92. FIG. 10D shows the diagram and sequence of BioE2022 (SEQ ID NO: 81) and BioE2023 (SEQ ID NO: 82) that form the ipi F(ab′)2. FIG. 10E shows the diagram and sequence of BioE2201 (SEQ ID NO: 83) and BioE2023 (SEQ ID NO: 82) that form the ipi Fab′. FIG. 10E discloses “His10” as SEQ ID NO: 92. FIG. 10F shows the diagram and sequence of BioE2033 (SEQ ID NO: 84) and BioE2034 (SEQ ID NO: 88) that form the 121 F(ab′)2. FIG. 10G shows the diagram and sequence of BioE2202 (SEQ ID NO: 86) and BioE2034 (SEQ ID NO: 88) that form the 121 Fab′. FIG. 10H shows the comparison of the C-terminal sequences of BioE2022 (SEQ ID NO: 91) and BioE2201 (SEQ ID NO: 92). FIG. 10I shows the comparison of the C-terminal sequences of BioE2033 (SEQ ID NO: 93) and BioE2202 (SEQ ID NO: 93).

FIG. 11 shows that targeting two epitopes on CTLA4 increases the CTLA4-blocking activity. The anti-CTLA4 diabodies (BioE2051 and BioE2052) have an increased CTLA-blocking activity as compared with ipilimumab or 121 HuIgG1.

FIG. 12 shows that targeting two epitopes on CTLA4 increases the CTLA4-blocking activity. The anti-CTLA4 diabody (BioE2052) or a combination of 121 scFv+Ipi scFv has an increased CTLA-blocking activity as compared with ipilimumab or 121 HuIgG1.

FIG. 13 shows that the anti-CTLA4 diabody (BioE2052) has an increased CTLA4-blocking activity as compared with ipilimumab or 121 HuIgG1.

FIG. 14 shows the increased CTLA4-blocking activity of the anti-CTLA4 diabody (BioE2052) as compared with ipilimumab.

FIG. 15 shows that the anti-CTLA4 diabody (BioE2052) has an increased CTLA4-blocking activity as compared with ipilimumab or 121 HuIgG1 (BioE2032).

FIG. 16 shows the CTLA4-blocking activity of ipilimumab, Ipi scFv, and Ipi F(ab′)2.

FIG. 17 shows the CTLA4-blocking activity of BioE2021 (Ipi scFv), BioE2031 (121 scFv), BioE2022 (Ipi F(ab′)2), BioE2033 (121 F(ab′)2), and ipilimumab.

FIG. 18 shows the CTLA4-blocking activity of the anti-CTLA4 diabody (BioE2052), a combination of BioE2021 (Ipi scFv)+BioE2031 (121 scFv), and a combination of BioE2022 (Ipi F(ab′)2)+BioE2033 (121 F(ab′)2).

FIG. 19 shows the CTLA4-blocking activity of the anti-CTLA4 diabody (BioE2052), BioE2201 (Ipi Fab′), BioE2202 (121 Fab′), a combination of BioE2201 (Ipi Fab′)+BioE2202 (121 Fab′), and ipilimumab.

FIG. 20 shows the CTLA4-blocking activity of the anti-CTLA4 diabody (BioE2052 (06966)), BioE2022 (Ipi F(ab′)2), BioE2033 (121 F(ab′)2), and ipilimumab done in triplicate and data rendered in ratios.

FIG. 21 shows the CTLA4-blocking activity of the CTLA4-binding proteins (BioE2051, BioE2052, BioE2081, BioE2082, BioE2091, BioE2092, BioE2121, BioE2012, and ipilimumab).

FIG. 22 shows the CTLA4-blocking activity of the CTLA4-binding proteins (BioE2021 Ipi ScFv, BioE2022 Ipi F(ab′)2, BioE2052 Ipi121-121Ipi, BioE2012, BioE2111 DART, and ipilimumab).

FIGS. 23A and 23B show the effects of BioE2052 and ipilimumab on mice body weight in the H22 model. FIG. 23A shows the effect of BioE2052 on body weight measured in grams. FIG. 23B shows the % change in body weight during treatment with BioE2052.

FIG. 24 shows the effects of administering BioE2052 and ipilimumab on tumor volume in the H22 Model

FIGS. 25A-25C show the changes in tumor volume in mice administered vehicle, BioE2052, or ipilimumab. FIG. 25A shows the changes in tumor volume in mice administered vehicle. FIG. 25B shows the changes in tumor volume in mice administered BioE2052. FIG. 25C shows the changes in tumor volume in mice administered ipilimumab.

FIG. 26 shows the peptide coverage, as determined by deuterium exchange, for CTLA4 bound by BioE2052 (SEQ ID NO: 97).

FIG. 27 shows a heatmap of residues in CTLA4 that are susceptible to deuterium exchange in the presence of BioE2052 (SEQ ID NO: 97).

FIG. 28A is a chromatogram of size exclusion HPLC performed on a sample of monoclonal anti-CTLA4 antibody BioE2420.

FIG. 28B is a chromatogram of a size exclusion HPLC performed on a sample of monoclonal anti-CTLA4 antibody BioE2430.

FIG. 29A summarizes the results of a CTLA4 Functional Blockade assay of BioE2420.

FIG. 29B summarizes the results of a CTLA4 Functional Blockade assay of BioE2430.

### DETAILED DESCRIPTION

Provided herein are engineered antigen-binding proteins that specifically bind to CTLA4 and show unexpected T cell activation properties. These proteins include those that bind to multiple epitopes on CTLA4, e.g., Epitope 1 (¹³⁴MYPPPY¹³⁹ (SEQ ID NO: 1)) and Epitope 2 (⁶⁵SICT⁶⁸ (SEQ ID NO: 2)), as well as F(ab′)2 or diabodies that are devoid of the Fc region of the antibody. The biparatopic entities presented herein showed unexpected synergistic effects on blocking CTLA4 ligand binding and T cell activation over the combination of monoparatopic counterparts (e.g., ipilimumab). In addition, the activity of the CTLA-binding proteins devoid of the Fc domain surpassed the activity of ipilimumab or other CTLA4-binding antibodies that comprise the Fc domain, which is contrary to the widely accepted belief that the interaction between the Fc region of a CTLA4 antibody with the Fc receptors is important for its CTLA4-blocking activity (Bulliard et al. (2013) J of Exp Medicine 9:1685-1693; Waight et al. (2018) Cancer Cell 33:1033-1047; Ingram et al. (2018) Proc Natl Acad Sci USA 115:3912-3917; Vargas et al. (2018) Cancer Cell 33:1-15). Thus, the engineered antigen-binding proteins and pharmaceutical compositions comprising same promise the clinical efficacy and safety that surpasses that of ipilimumab. Further provided herein are nucleic acids, vectors, host cells, conjugates, kits, pharmaceutical compositions, and methods of making the engineered antigen-binding proteins. Moreover, methods of treating a patient (e.g., those afflicted with a cancer or other diseases that are characterized by increased CTLA4 expression and/or activity), methods of increasing immune response, methods of reducing proliferation of a cancer cell, and methods of activating T cells are presented herein.

### Definitions

The articles “a” and “an” are used herein to refer to one or to more than one (i.e. to at least one) of the grammatical object of the article. By way of example, “an element” means one element or more than one element.

The term “antigen presenting cell” includes professional antigen presenting cells (e.g., B lymphocytes, monocytes, dendritic cells, Langerhans cells) as well as other antigen presenting cells (e.g., keratinocytes, endothelial cells, astrocytes, fibroblasts, oligodendrocytes).

As used herein, the term “composite antibody” refers to an antibody which has variable regions comprising germline or non-germline immunoglobulin sequences from two or more unrelated variable regions. Additionally, the term “composite, human antibody” refers to an antibody which has constant regions derived from human germline or non-germline immunoglobulin sequences and variable regions comprising human germline or non-germline sequences from two or more unrelated human variable regions.

The terms “conjoint therapy” and “combination therapy,” as used herein, refer to the administration of two or more therapeutic substances. The different agents comprising the combination therapy may be administered concomitant with, prior to, or following the administration of one or more therapeutic agents.

By “detectable label” is meant a compound, substance, or composition that, when linked to a molecule of interest, renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

As used herein, the term “Fc region” is used to describe a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. Suitable native-sequence Fc regions for use in the antibodies of the present invention include human IgG1, IgG2 (IgG2A, IgG2B), IgG3 and IgG4.

As used herein, “Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see M. Daëron, Annu. Rev. Immunol. 15:203-234 (1997). FcRs are reviewed in Ravetch and Kinet, Annu. Rev. Immunol. 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

Antibodies may also be “humanized”, which is intended to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies of the present invention may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, such as a mouse, have been grafted onto human framework sequences.

“Interleukin-2 (IL-2)” is a cytokine signaling molecule that functions in the immune system. The IL-2 protein is produced primarily by activated T cells (CD4+ T cells); it regulates the activities of other T cells and B cells (increases growth and activity of these white blood cells) that are responsible for immunity. IL-2 is classified as a biologic response modifier that can modify the body's response to cancer cells. The production of IL-2 exerts a wide spectrum of immunoregulatory effects on the immune system, e.g., increasing the proliferation and/or functional activity of other immune cells, such as tumor-infiltrating lymphocytes (TILs; T cells) and natural killer (NK) cells, enhancement of lymphocyte mitogenesis, lymphocyte cytotoxicity, induction of NK cells and lymphokine activated NK cells, and induction of interferon-γ production (S. L. Gaffen et al., 2004, Cytokine, 28 (3): 109-23). In T cells, IL-2 synthesis is tightly regulated at the mRNA level by signals from the T cell receptor (TCR) and CD28.

As used herein, the term “KD” is intended to refer to the dissociation equilibrium constant of a particular antibody-antigen interaction. The binding affinity of antibodies of the disclosed invention may be measured or determined by standard antibody-antigen assays, for example, competitive assays, saturation assays, or standard immunoassays such as ELISA or RIA.

A nucleic acid is “operably linked” when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination.

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The term “remission” is art recognized, and refers to a condition in which the signs and symptoms of the cancer are reduced.

The term “selective” refers to a preferential action or function. The term “selective” can be quantified in terms of the preferential effect in a particular target of interest relative to other targets. For example, a measured variable (e.g., binding of the CTLA4-binding protein or the CTLA4-blocking activity) can be 10%, 20%, 25%, 30%, 35%, 40%, 45%, 50%, 55%, 60%, 65%, 70%, 75%, 80%, 85%, 90%, 95%, 1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 25-fold, 30-fold, 35-fold, 40-fold, 45-fold, 50-fold, 55-fold, 60-fold, 70-fold, 80-fold, 90-fold, 100-fold, or greater or any range in between inclusive (e.g., 50% to 16-fold), different in a target of interest versus unintended or undesired targets. The same fold analysis can be used to confirm the magnitude of an effect in a given tissue, cell population, measured variable, measured effect, and the like.

By contrast, the term “specific” refers to an exclusionary action or function. For example, specific binding of an antibody or antigen-binding protein to a predetermined antigen refers to the ability of the antibody or antigen-binding protein to bind to the antigen of interest without binding to other antigens. Typically, the antibody binds with an affinity (K_{D}) of approximately less than 1×10⁻⁷ M, such as approximately less than 10⁻⁸ M, 10⁻⁹ M, 10⁻¹⁰ M, 10⁻¹¹ M, or even lower to the predetermined antigen with an affinity that is at least 1.1-, 1.2-, 1.3-, 1.4-, 1.5-, 1.6-, 1.7-, 1.8-, 1.9-, 2.0-, 2.5-, 3.0-, 3.5-, 4.0-, 4.5-, 5.0-, 6.0-, 7.0-, 8.0-, 9.0-, or 10.0-fold or greater than its affinity for binding to a non-specific antigen (e.g., BSA, casein) other than the predetermined antigen or a closely-related antigen.

The term “sensitize” means to alter cells, such as cancer cells or tumor cells, in a way that allows for more effective treatment with a therapy (e.g., a CTLA4-binding protein). In some embodiments, normal cells are not affected to an extent that causes the normal cells to be unduly injured by the therapy (e.g., a CTLA4-binding protein). An increased sensitivity or a reduced sensitivity to a therapeutic treatment is measured according to a known method in the art for the particular treatment and methods described herein below, including, but not limited to, cell proliferative assays (Tanigawa N, Kern D H, Kikasa Y, Morton D L, Cancer Res 1982; 42:2159-2164), cell death assays (Weisenthal L M, Shoemaker R H, Marsden J A, Dill P L, Baker J A, Moran E M, Cancer Res 1984; 94:161-173; Weisenthal L M, Lippman M E, Cancer Treat Rep 1985; 69:615-632; Weisenthal L M, In: Kaspers G J L, Pieters R, Twentyman P R, Weisenthal L M, Veerman A J P, eds. Drug Resistance in Leukemia and Lymphoma. Langhorne, P A: Harwood Academic Publishers, 1993:415-432; Weisenthal L M, Contrib Gynecol Obstet 1994; 19:82-90). The sensitivity or resistance may also be measured in animal by measuring the tumor size reduction over a period of time, for example, 6 months for human and 4-6 weeks for mouse. A composition or a method sensitizes response to a therapeutic treatment if the increase in treatment sensitivity or the reduction in resistance is 5% or more, for example, 10%, 15%, 20%, 25%, 30%, 35%, 40%, 45%, 50%, 55%, 60%, 65%, 70%, 75%, 80%, 85%, 90%, 95%, 100%, or more, to 2-fold, 3-fold, 4-fold, 5-fold, 10-fold, 15-fold, 20-fold or more, compared to treatment sensitivity or resistance in the absence of such composition or method. The determination of sensitivity or resistance to a therapeutic treatment is routine in the art and within the skill of an ordinarily skilled clinician. It is to be understood that any method described herein for enhancing the efficacy of a CTLA4-binding protein, can be equally applied to methods for sensitizing hyperproliferative or otherwise cancerous cells (e.g., resistant cells) to the therapy.

As used herein, “subject” refers to any healthy animal, mammal or human, or any animal, mammal or human afflicted with a cancer. The term “subject” is interchangeable with “patient.”

The term “synergistic effect” refers to the combined effect of two or more therapeutic agents, such as two or more CTLA4 pathway modulators, either alone or in combination with another cancer therapy can be greater than the sum of the separate effects of individual agents alone. As used herein, the synergistic effect may also be used to refer to the effect of a single CTLA4-binding protein that comprises two or more binding moieties, wherein the effect (e.g., biological effect or therapeutic effect) is greater than the sum of the separate effects of the individual binding moieties.

Conventional T cells, also known as Tcons or Teffs, have effector functions (e.g., cytokine secretion, cytotoxic activity, anti-self-recognization, and the like) to increase immune responses by virtue of their expression of one or more T cell receptors. Tcons or Teffs are generally defined as any T cell population that is not a Treg and include, for example, naïve T cells, activated T cells, memory T cells, resting Tcons, or Tcons that have differentiated toward, for example, the Th1 or Th2 lineages. In some embodiments, Teffs are a subset of non-Treg T cells. In some embodiments, Teffs are CD4+Teffs or CD8+Teffs, such as CD4+ helper T lymphocytes (e.g., Th0, Th1, Tfh, or Th17) and CD8+ cytotoxic T lymphocytes. As described further herein, cytotoxic T cells are CD8+T lymphocytes. “Naïve Tcons” are CD4+ T cells that have differentiated in bone marrow, and successfully underwent a positive and negative processes of central selection in a thymus, but have not yet been activated by exposure to an antigen. Naïve Tcons are commonly characterized by surface expression of L-selectin (CD62L), absence of activation markers such as CD25, CD44 or CD69, and absence of memory markers such as CD45RO. Naïve Tcons are therefore believed to be quiescent and non-dividing, requiring interleukin-7 (IL-7) and interleukin-15 (IL-15) for homeostatic survival (see, at least WO 2010/101870). The presence and activity of such cells are undesired in the context of suppressing immune responses. Unlike Tregs, Tcons are not anergic and can proliferate in response to antigen-based T cell receptor activation (Lechler et al. (2001) Philos. Trans. R. Soc. Lond. Biol. Sci. 356:625-637). In tumors, exhausted cells can present hallmarks of anergy.

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human.

The terms “therapeutically-effective amount” and “effective amount” as used herein means that amount of a compound, material, or composition comprising a compound encompassed by the present disclosure which is effective for producing some desired therapeutic effect in at least a sub-population of cells in an animal at a reasonable benefit/risk ratio applicable to any medical treatment. Toxicity and therapeutic efficacy of subject compounds may be determined by standard pharmaceutical procedures in cell cultures or experimental animals, e.g., for determining the LD₅₀ and the ED₅₀. Compositions that exhibit large therapeutic indices are preferred. In some embodiments, the LD₅₀ (lethal dosage) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more reduced for the agent relative to no administration of the agent. Similarly, the ED₅₀ (i.e., the concentration which achieves a half-maximal inhibition of symptoms) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. Also, similarly, the IC₅₀ (i.e., the concentration which achieves half-maximal cytotoxic or cytostatic effect on cancer cells) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. In some embodiments, cancer cell growth in an assay can be inhibited by at least about 10%, 15%, 20%, 25%, 30%, 35%, 40%, 45%, 50%, 55%, 60%, 65%, 70%, 75%, 80%, 85%, 90%, 95%, or even 100%. Cancer cell death can be promoted by at least about 10%, 15%, 20%, 25%, 30%, 35%, 40%, 45%, 50%, 55%, 60%, 65%, 70%, 75%, 80%, 85%, 90%, 95%, or even 100%. In another embodiment, at least about a 10%, 15%, 20%, 25%, 30%, 35%, 40%, 45%, 50%, 55%, 60%, 65%, 70%, 75%, 80%, 85%, 90%, 95%, or even 100% decrease in cancer cell numbers and/or a solid malignancy can be achieved.

### CTLA4, Epitopes, and Ipilimumab

T cells, both CD4 (helper) and CD8 (cytotoxic), contribute to the adaptive immune response against pathogens and tumors, and activation and recruitment of specific T cells constitute a complex process. For a T cell to become fully activated (and subsequently proliferate and mediate effector function), at least 2 receptor-ligand interactions are required. The first of these occurs when the unique receptor of the T cell recognizes its cognate ligand, a short peptide presented in the context of a MHC molecule. This interaction is exquisitely specific, and if a good fit occurs, T-cell activation is initiated. However, full activation of a CD4 or CD8 T cell requires a second signal transmitted by costimulatory molecules present on the same antigen-presenting cell that expresses the peptide/MHC. This second signal is transmitted from costimulatory molecules (B7-1 (CD80) and/or B7-2 (CD86)) to a receptor on T cells known as CD28. Only when both signals are received and integrated does a specific T cell proliferate, acquire effector function, and migrate to sites of antigen expression.

CTLA4 is a homolog of CD28, suggesting that CTLA4 might serve, along with CD28, as a costimulatory molecule. However, several other studies provided opposing results, and for some time, it was not clear whether CTLA4 transmitted a stimulatory or inhibitory signal to T cells. The generation of mice lacking CTLA4 provided a solution for this conundrum: Knockout mice developed a progressive accumulation of activated T cells and died of lymphoproliferative disease ˜3 to 4 weeks after birth. These and other results suggested that blockade of CTLA4 with a monoclonal antibody could augment an adaptive immune response to an infectious agent or an evolving tumor. The subsequent study showed that CTLA4 blockade could attenuate the growth of several implanted murine tumors.

CTLA4 (also known as CD152) is a protein receptor that functions as an immune checkpoint and downregulates immune responses. CTLA4 is constitutively expressed in regulatory T cells but only upregulated in conventional T cells after activation—a phenomenon which is particularly notable in cancers. It acts as an “off” switch when bound to CD80 or CD86 on the surface of antigen-presenting cells. Thus, CTLA4 is a member of the immunoglobulin superfamily and encodes a protein which transmits an inhibitory signal to T cells.

The protein contains an extracellular V domain, a transmembrane domain, and a cytoplasmic tail. Alternate transcriptional splice variants, encoding different isoforms, have been characterized. The membrane-bound isoform functions as a homodimer interconnected by a disulfide bond, while the soluble isoform functions as a monomer. The intracellular domain is similar to that of CD28, in that it has no intrinsic catalytic activity and contains one YVKM motif (SEQ ID NO: 73) able to bind PI3K, PP2A and SHP-2 and one proline-rich motif able to bind SH3 containing proteins. The first role of CTLA4 in inhibiting T cell responses seem to be directly via SHP-2 and PP2A dephosphorylation of TCR-proximal signaling proteins such as CD3 and LAT. CTLA4 can also affect signaling indirectly via competing with CD28 for CD80/86 binding. CTLA4 can also bind PI3K, although the importance and results of this interaction are uncertain. CTLA4 is known to interact with various proteins such as CD80, CD86, CTXN3, MALL, PIK3R1, and TMEM218.

Mutations in CTLA4 have been associated with insulin-dependent diabetes mellitus, Graves' disease, Hashimoto thyroiditis, celiac disease, systemic lupus erythematosus, thyroid-associated orbitopathy, and other autoimmune diseases.

The nucleic acids and polypeptide sequences of CTLA4 in humans and other organisms are well-known and include, for example, human CTLA4 (NM_001037631.3→NP_001032720.1 cytotoxic T-lymphocyte protein 4 isoform CTLA-4delTM; NM_005214.5→NP_005205.2 cytotoxic T-lymphocyte protein 4 isoform CTLA4-TM precursor), mouse CTLA4 (NM_001281976.1→NP_001268905.1 cytotoxic T-lymphocyte protein 4 isoform 2 precursor; NM_009843.4→NP_033973.2 cytotoxic T-lymphocyte protein 4 isoform 1 precursor), and rat (NM_031674.1→NP_113862.1 cytotoxic T-lymphocyte protein 4 precursor). Representative nucleic acid and polypeptide sequences are disclosed in Table 1 below.

TABLE 1
Exemplary sequence of CTLA4 and its epitopes

```
SEQ ID NO: 1
Epitope 1 of CTLA4 (amino acid residues 134-139 of CTLA4)
    1 mypppy
SEQ ID NO: 2
Epitope 2 of CTLA4 (amino acid residues 65-68 of CTLA4)
    1 sict
* The amino acid residues of 65-68 of ⁶⁵SICT⁶⁸ (SEQ ID NO: 2) correspond to the amino
acid residues of the mature CTLA4 polypeptide. SEQ ID NO: 3 is the sequence of the
CTLA4 polypeptide before maturation, thus the SICT epitope (SEQ ID NO: 2) corresponds
to the amino acid residues 101-104 of SEQ ID NO: 3.
SEQ ID NO: 3
Human CTLA4 isoform CTLA4-TM amino acid sequence
(NP_005205.2)
      1 maclgfqrhk aqlnlatrtw pctllffllf ipvfckamhv aqpavvlass rgiasfvcey
    61 aspgkatevr vtvlrqadsq vtevcaatym mgneltfldd sictgtssgn qvnltiqglr
  121 amdtglyick velmypppyy lgigngtqiy vidpepcpds dfllwilaav ssglffysfl
  181 ltavslskml kkrsplttgv yvkmpptepe cekqfqpyfi pin
Human CTLA4 transcript variant 1 cDNA sequence
SEQ ID NO: 4 Human CTLA4 transcript variant 1 cDNA sequence
(NM_005214.5; CDS 173-844)
      1 gctttctatt caagtgcctt ctgtgtgtgc acatgtgtaa tacatatctg ggatcaaagc
    61 tatctatata aagtccttga ttctgtgtgg gttcaaacac atttcaaagc ttcaggatcc
  121 tgaaaggttt tgctctactt cctgaagacc tgaacaccgc tcccataaag ccatggcttg
  181 ccttggattt cagcggcaca aggctcagct gaacctggct accaggacct ggccctgcac
  241 tctcctgttt tttcttctct tcatccctgt cttctgcaaa gcaatgcacg tggcccagcc
  301 tgctgtggta ctggccagca gccgaggcat cgccagcttt gtgtgtgagt atgcatctcc
  361 aggcaaagcc actgaggtcc gggtgacagt gcttcggcag gctgacagcc aggtgactga
  421 agtctgtgcg gcaacctaca tgatggggaa tgagttgacc ttcctagatg attccatctg
  481 cacgggcacc tccagtggaa atcaagtgaa cctcactatc caaggactga gggccatgga
  541 cacgggactc tacatctgca aggtggagct catgtaccca ccgccatact acctgggcat
  601 aggcaacgga acccagattt atgtaattga tccagaaccg tgcccagatt ctgacttcct
  661 cctctggatc cttgcagcag ttagttcggg gttgtttttt tatagctttc tcctcacagc
  721 tgtttctttg agcaaaatgc taaagaaaag aagccctctt acaacagggg tctatgtgaa
  781 aatgccccca acagagccag aatgtgaaaa gcaatttcag ccttatttta ttcccatcaa
  841 ttgagaaacc attatgaaga agagagtcca tatttcaatt tccaagagct gaggcaattc
  901 taactttttt gctatccagc tatttttatt tgtttgtgca tttgggggga attcatctct
  961 ctttaatata aagttggatg cggaacccaa attacgtgta ctacaattta aagcaaagga
1021 gtagaaagac agagctggga tgtttctgtc acatcagctc cactttcagt gaaagcatca
1081 cttgggatta atatggggat gcagcattat gatgtgggtc aaggaattaa gttagggaat
1141 ggcacagccc aaagaaggaa aaggcaggga gcgagggaga agactatatt gtacacacct
1201 tatatttacg tatgagacgt ttatagccga aatgatcttt tcaagttaaa ttttatgcct
1261 tttatttctt aaacaaatgt atgattacat caaggcttca aaaatactca catggctatg
1321 ttttagccag tgatgctaaa ggttgtattg catatataca tatatatata tatatatata
1381 tatatatata tatatatata tatatatata tatattttaa tttgatagta ttgtgcatag
1441 agccacgtat gtttttgtgt atttgttaat ggtttgaata taaacactat atggcagtgt
1501 ctttccacct tgggtcccag ggaagttttg tggaggagct caggacacta atacaccagg
1561 tagaacacaa ggtcatttgc taactagctt ggaaactgga tgaggtcata gcagtgcttg
1621 attgcgtgga attgtgctga gttggtgttg acatgtgctt tggggctttt acaccagttc
1681 ctttcaatgg tttgcaagga agccacagct ggtggtatct gagttgactt gacagaacac
1741 tgtcttgaag acaatggctt actccaggag acccacaggt atgaccttct aggaagctcc
1801 agttcgatgg gcccaattct tacaaacatg tggttaatgc catggacaga agaaggcagc
1861 aggtggcaga atggggtgca tgaaggtttc tgaaaattaa cactgcttgt gtttttaact
1921 caatattttc catgaaaatg caacaacatg tataatattt ttaattaaat aaaaatctgt
1981 ggtggtcgtt ttccgga
SEQ ID NO: 5
Human CTLA4 isoform CTLA-4delTM amino acid sequence
(NP_001032720.1)
      1 maclgfqrhk aqlnlatrtw pctllffllf ipvfckamhv aqpavvlass rgiasfvcey
    61 aspgkatevr vtvlrqadsq vtevcaatym mgneltfldd sictgtssgn qvnltiqglr
  121 amdtglyick velmypppyy lgigngtqiy viakekkpsy nrglcenapn rarm
SEQ ID NO: 6 Human CTLA4 transcript variant 2 cDNA sequence
(NM_001037631.3; CDS 173-697)
      1 gctttctatt caagtgcctt ctgtgtgtgc acatgtgtaa tacatatctg ggatcaaagc
    61 tatctatata aagtccttga ttctgtgtgg gttcaaacac atttcaaagc ttcaggatcc
  121 tgaaaggttt tgctctactt cctgaagacc tgaacaccgc toccataaag ccatggcttg
  181 ccttggattt cagcggcaca aggctcagct gaacctggct accaggacct ggccctgcac
  241 tctcctgttt tttcttctct tcatccctgt cttctgcaaa gcaatgcacg tggcccagcc
  301 tgctgtggta ctggccagca gccgaggcat cgccagcttt gtgtgtgagt atgcatctcc
  361 aggcaaagcc actgaggtcc gggtgacagt gcttcggcag gctgacagcc aggtgactga
  421 agtctgtgcg gcaacctaca tgatggggaa tgagttgacc ttcctagatg attccatctg
  481 cacgggcacc tccagtggaa atcaagtgaa cctcactatc caaggactga gggccatgga
  541 cacgggactc tacatctgca aggtggagct catgtaccca ccgccatact acctgggcat
  601 aggcaacgga acccagattt atgtaattgc taaagaaaag aagccctctt acaacagggg
  661 tctatgtgaa aatgccccca acagagccag aatgtgaaaa gcaatttcag ccttatttta
  721 ttcccatcaa ttgagaaacc attatgaaga agagagtcca tatttcaatt tccaagagct
  781 gaggcaattc taactttttt gctatccagc tatttttatt tgtttgtgca tttgggggga
  841 attcatctct ctttaatata aagttggatg cggaacccaa attacgtgta ctacaattta
  901 aagcaaagga gtagaaagac agagctggga tgtttctgtc acatcagctc cactttcagt
  961 gaaagcatca cttgggatta atatggggat gcagcattat gatgtgggtc aaggaattaa
1021 gttagggaat ggcacagccc aaagaaggaa aaggcaggga gcgagggaga agactatatt
1081 gtacacacct tatatttacg tatgagacgt ttatagccga aatgatcttt tcaagttaaa
1141 ttttatgcct tttatttctt aaacaaatgt atgattacat caaggcttca aaaatactca
1201 catggctatg ttttagccag tgatgctaaa ggttgtattg catatataca tatatatata
1261 tatatatata tatatatata tatatatata tatatatata tatattttaa tttgatagta
1321 ttgtgcatag agccacgtat gtttttgtgt atttgttaat ggtttgaata taaacactat
1381 atggcagtgt ctttccacct tgggtcccag ggaagttttg tggaggagct caggacacta
1441 atacaccagg tagaacacaa ggtcatttgc taactagctt ggaaactgga tgaggtcata
1501 gcagtgcttg attgcgtgga attgtgctga gttggtgttg acatgtgctt tggggctttt
1561 acaccagttc ctttcaatgg tttgcaagga agccacagct ggtggtatct gagttgactt
1621 gacagaacac tgtcttgaag acaatggctt actccaggag acccacaggt atgaccttct
1681 aggaagctcc agttcgatgg gcccaattct tacaaacatg tggttaatgc catggacaga
1741 agaaggcagc aggtggcaga atggggtgca tgaaggtttc tgaaaattaa cactgcttgt
1801 gtttttaact caatattttc catgaaaatg caacaacatg tataatattt ttaattaaat
1861 aaaaatctgt ggtggtcgtt ttccgga
SEQ ID NO: 7
Mouse CTLA4 isoform 1 amino acid sequence (NP_033973.2)
      1 maclglrryk aqlqlpsrtw pfvalltllf ipvfseaiqv tqpsvvlass hgvasfpcey
    61 spshntdevr vtvlrqtndq mtevcattft ekntvgfldy pfcsgtfnes rvnltiqglr
  121 avdtglylck velmypppyf vgmgngtqiy vidpepcpds dfllwilvav slglffysfl
  181 vtavslskml kkrsplttgv yvkmpptepe cekqfqpyfi pin
SEQ ID NO: 8
Mouse CTLA4 transcript variant 1 cDNA sequence
(NM_009843.4; CDS 147-818)
      1 ctacacatat gtagcacgta ccttggatca aagctgtcta tataaagtcc ccgagtctgt
    61 gtgggttcaa acacatctca aggcttctgg atcctgttgg gttttactct gctccctgag
  121 gacctcagca catttgcccc ccagccatgg cttgtcttgg actccggagg tacaaagctc
  181 aactgcagct gccttctagg acttggcctt ttgtagccct gctcactctt cttttcatcc
  241 cagtcttctc tgaagccata caggtgaccc aaccttcagt ggtgttggct agcagccatg
  301 gtgtcgccag ctttccatgt gaatattcac catcacacaa cactgatgag gtccgggtga
  361 ctgtgctgcg gcagacaaat gaccaaatga ctgaggtctg tgccacgaca ttcacagaga
  421 agaatacagt gggcttccta gattacccct tctgcagtgg tacctttaat gaaagcagag
  481 tgaacctcac catccaagga ctgagagctg ttgacacggg actgtacctc tgcaaggtgg
  541 aactcatgta cccaccgcca tactttgtgg gcatgggcaa cgggacgcag atttatgtca
  601 ttgatccaga accatgcccg gattctgact tcctcctttg gatccttgtc gcagttagct
  661 tggggttgtt tttttacagt ttcctggtca ctgctgtttc tttgagcaag atgctaaaga
  721 aaagaagtcc tottacaaca ggggtctatg tgaaaatgcc cccaacagag ccagaatgtg
  781 aaaagcaatt tcagccttat tttattccca tcaactgaaa ggccgtttat gaagaagaag
  841 gagcatactt cagtctctaa aagctgaggc aatttcaact ttccttttct ctccagctat
  901 ttttacctgt ttgtatattt taaggagagt atgcctctct ttaatagaaa gctggatgca
  961 aaattccaat taagcatact acaatttaaa gctaaggagc atgaacagag agctgggata
1021 tttctgttgt gtcagaacca ttttactaaa agcatcactt ggaagcagca taaggatata
1081 gcattatggt gtggggtcaa gggaacatta gggaatggca cagcccaaag aaaggaaggg
1141 ggtgaaggaa gagattatat tgtacacatc ttgtatttac ctgagagatg tttatgactt
1201 aaataatttt taaatttttc atgctgttat tttctttaac aatgtataat tacacgaagg
1261 tttaaacatt tattcacaga gctatgtgac atagccagtg gttccaaagg ttgtagtgtt
1321 ccaagatgta tttttaagta atattgtaca tgggtgtttc atgtgctgtt gtgtatttgc
1381 tggtggtttg aatataaaca ctatgtatca gtgtcgtccc acagtgggtc ctggggaggt
1441 ttggctgggg agcttaggac actaatccat caggttggac tcgaggtcct gcaccaactg
1501 gcttggaaac tagatgaggc tgtcacaggg ctcagttgca taaaccgatg gtgatggagt
1561 gtaaactggg tctttacact cattttattt tttgtttctg cttttgtttt cttcaatgat
1621 ttgcaaggaa accaaaagct ggcagtgttt gtatgaacct gacagaacac tgtcttcaag
1681 gaaatgcctc attcctgaga ccagtaggtt tgttttttta ggaagttcca atactaggac
1741 cccctacaag tactatggct cctcgaaaac acaaagttaa tgccacagga agcagcagat
1801 ggtaggatgg gatgcacaag agttcctgaa aactaacact gttagtgttt tttttttaac
1861 tcaatatttt ccatgaaaat gcaaccacat gtataatatt tttaattaaa taaaagtttc
1921 ttgtgattgt ttt
*Included in Table 1 are RNA nucleic acid molecules (e.g., thymidines replaced with uridines), nucleic acid molecules encoding orthologs of the encoded proteins, as well as DNA, cDNA, or RNA nucleic acid sequences comprising a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any SEQ ID NO listed in Table 1, or a portion thereof. Such nucleic acid molecules can have a function of the full-length nucleic acid.
*Included in Table 1 are orthologs of the proteins, as well as polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any SEQ ID NO listed in Table 1, or a portion thereof. Such polypeptides can have a function of the full-length polypeptide.
*Included in Table 1 are other known CTLA4 nucleic acid and amino acid sequences.
```

Ipilimumab, sold under the brand name Yervoy, is a monoclonal antibody medication that works to activate the immune system by targeting CTLA4, a protein receptor that downregulates the immune system. Ipilimumab blocks the interaction between CTLA4 and its ligands. As described above, cytotoxic T lymphocytes (CTLs) can recognize and destroy cancer cells. However, an inhibitory mechanism interrupts this destruction. Ipilimumab turns off this inhibitory mechanism and boosts the body's immune response against cancer cells.

Ipilimumab binds to Epitope 1 (¹³⁴MYPPPY¹³⁹ (SEQ ID NO: 1)) of CTLA4. The MYPPPY motif (SEQ ID NO: 1), including Tyr139, is highly conserved across both CTLA4 and its immune stimulatory paralog CD28, and interfaces directly with both CD80 and with the CTLA4 inhibitor ipilimumab. Other antibodies, e.g., murine 26 antibody or humanized 121 antibody described herein and in U.S. Pat. No. 7,034,121 B2 bind to Epitope 2 (⁶⁵SICT⁶⁸ (SEQ ID NO: 2) of CTLA4. The significance of these epitopes were unknown prior to this disclosure.

Ipilimumab was approved by the US Food and Drug Administration (FDA) for treatment of melanoma (e.g., unresectable or metastatic melanoma in adults and pediatric patients), renal cell carcinoma (RCC), colorectal cancer, hepatocellular carcinoma, non-small cell lung cancer (NSCLC), and malignant pleural mesothelioma. Ipilimumab is also effective in combination with nivolumab that targets PD-1.

While effective, a major drawback of ipilimumab therapy is its association with severe and potentially fatal immunological adverse effects due to T cell activation and proliferation, occurring in ten to twenty percent of patients. Serious adverse effects include stomach pain, bloating, constipation, diarrhea, fever, trouble breathing, and urinating problems. Between 5.7 and 9.1% of individuals treated with ipilimumab develop checkpoint inhibitor induced colitis. Individual cases of severe neurologic disorders following ipilimumab have been observed, including acute inflammatory demyelination polyneuropathy and an ascending motor paralysis, and myasthenia gravis.

### Antigen-Binding Proteins

Provided herein are antigen-binding proteins that bind to CTLA4. The antigen-binding proteins of the present disclosure can take any one of many forms of antigen-binding proteins known in the art. In various embodiments, the antigen-binding proteins of the present disclosure take the form of an antibody, or antigen-binding antibody fragment, or an antibody protein product.

In various embodiments of the present disclosure, the antigen-binding protein comprises, consists essentially of, or consists of an antibody or a fragment thereof. As used herein, the term “antibody” refers to a protein having a conventional immunoglobulin format, comprising heavy and light chains, and comprising variable and constant regions. For example, an antibody may be an IgG which is a “Y-shaped” structure of two identical pairs of polypeptide chains, each pair having one “light” (typically having a molecular weight of about 25 kDa) and one “heavy” chain (typically having a molecular weight of about 50-70 kDa). An antibody has a variable region and a constant region. In IgG formats, the variable region is generally about 100-110 or more amino acids, comprises three complementarity determining regions (CDRs), is primarily responsible for antigen recognition, and substantially varies among other antibodies that bind to different antigens. Antibody-based antigen-binding proteins comprise the CDRs of the antibody, but not necessarily other regions (e.g., the constant region). The constant region allows the antibody to recruit cells and molecules of the immune system. The variable region is made of the N-terminal regions of each light chain and heavy chain, while the constant region is made of the C-terminal portions of each of the heavy and light chains. (Janeway et al., “Structure of the Antibody Molecule and the Immunoglobulin Genes”, Immunobiology: The Immune System in Health and Disease, 4th ed. Elsevier Science Ltd./Garland Publishing, (1999)).

The general structure and properties of CDRs of antibodies have been described in the art. Briefly, in an antibody scaffold, the CDRs are embedded within a framework in the heavy and light chain variable region where they constitute the regions largely responsible for antigen binding and recognition. A variable region typically comprises at least three heavy or light chain CDRs (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342:877-883), within a framework region (designated framework regions 1-4, FR1, FR2, FR3, and FR4, by Kabat et al., 1991; see also Chothia and Lesk, 1987, supra).

CDR refers to a complementarity determining region (CDR) of which three make up the binding character of a light chain variable region (CDR-L1, CDR-L2 and CDR-L3) and three make up the binding character of a heavy chain variable region (CDR-H1, CDR-H2 and CDR-H3). CDRs contribute to the functional activity of an antibody molecule and are separated by amino acid sequences that comprise scaffolding or framework regions. The exact definitional CDR boundaries and lengths are subject to different classification and numbering systems. CDRs may therefore be referred to by Kabat, Chothia, contact or any other boundary definitions. Despite differing boundaries, each of these systems has some degree of overlap in what constitutes the so called “hypervariable regions” within the variable sequences. CDR definitions according to these systems may therefore differ in length and boundary areas with respect to the adjacent framework region. See for example Kabat, Chothia, and/or MacCallum et al., (Kabat et al., in “Sequences of Proteins of Immunological Interest,” 5th Edition, U.S. Department of Health and Human Services, 1992; Chothia et al. (1987) J. Mol. Biol. 196, 901; and MacCallum et al., J. Mol. Biol. (1996) 262, 732, each of which is incorporated by reference in its entirety).

Antibodies can comprise any constant region known in the art. Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Embodiments of the present disclosure include all such classes or isotypes of antibodies. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. Accordingly, in various embodiments, the antibody is an antibody of isotype IgA, IgD, IgE, IgG, or IgM, including any one of IgG1, IgG2, IgG3 or IgG4. In various aspects, the antibody comprises a constant region comprising one or more amino acid modifications, relative to the naturally-occurring counterpart, in order to improve half-life/stability or to render the antibody more suitable for expression/manufacturability. In various instances, the antibody comprises a constant region wherein the C-terminal Lys residue that is present in the naturally-occurring counterpart is removed or clipped.

The antibody can be a monoclonal antibody. In some embodiments, the antibody comprises a sequence that is substantially similar to a naturally-occurring antibody produced by a mammal, e.g., mouse, rabbit, goat, horse, chicken, hamster, human, and the like. In this regard, the antibody can be considered as a mammalian antibody, e.g., a mouse antibody, rabbit antibody, goat antibody, horse antibody, chicken antibody, hamster antibody, human antibody, and the like. In certain aspects, the antigen-binding protein is an antibody, such as a human antibody. In certain aspects, the antigen-binding protein is a chimeric antibody or a humanized antibody. The term “chimeric antibody” refers to an antibody containing domains from two or more different antibodies. A chimeric antibody can, for example, contain the constant domains from one species and the variable domains from a second, or more generally, can contain stretches of amino acid sequence from at least two species. A chimeric antibody also can contain domains of two or more different antibodies within the same species. The term “humanized” when used in relation to antibodies refers to antibodies having at least CDR regions from a non-human source which are engineered to have a structure and immunological function more similar to true human antibodies than the original source antibodies. For example, humanizing can involve grafting a CDR from a non-human antibody, such as a mouse antibody, into a human antibody. Humanizing also can involve select amino acid substitutions to make a non-human sequence more similar to a human sequence. Information, including sequence information for human antibody heavy and light chain constant regions is publicly available through the Uniprot database as well as other databases well-known to those in the field of antibody engineering and production. For example, the IgG2 constant region is available from the Uniprot database as Uniprot number P01859, incorporated herein by reference.

An antibody can be cleaved into fragments by enzymes, such as, e.g., papain and pepsin. Papain cleaves an antibody to produce two Fab′ fragments and a single Fc fragment. Pepsin cleaves an antibody to produce a F(ab′)2 fragment and a pFc′ fragment. In various aspects of the present disclosure, the antigen-binding protein of the present disclosure is an antigen-binding fragment of an antibody (a.k.a., antigen-binding antibody fragment, antigen-binding fragment, antigen-binding portion). In various instances, the antigen-binding antibody fragment is a Fab′ fragment or a F(ab′)2 fragment.

The architecture of antibodies has been exploited to create a growing range of alternative antibody formats that spans a molecular-weight range of at least about 12-150 kDa and has a valency (n) range from monomeric (n=1), to dimeric (n=2), to trimeric (n=3), to tetrameric (n=4), and potentially higher; such alternative antibody formats are referred to herein as “antibody protein products.” Antibody protein products include those based on the full antibody structure and those that mimic antibody fragments which retain full antigen-binding capacity, e.g., scFvs, Fabs and VHH/VH (discussed below). The smallest antigen-binding fragment that retains its complete antigen binding site is the Fv fragment, which consists entirely of variable (V) regions. A soluble, flexible amino acid peptide linker is used to connect the V regions to a scFv (single chain fragment variable) fragment for stabilization of the molecule, or the constant (C) domains are added to the V regions to generate a Fab′ fragment. Both scFv and Fab′ fragments can be easily produced in host cells, e.g., prokaryotic host cells. Other antibody protein products include disulfide-bond stabilized scFv (ds-scFv), single chain Fab′ (scFab′), as well as di- and multimeric antibody formats like dia-, tria- and tetra-bodies, or minibodies (miniAbs) that comprise different formats consisting of scFvs linked to oligomerization domains. The smallest fragments are VHH/VH of camelid heavy chain Abs as well as single domain Abs (sdAb). The building block that is most frequently used to create novel antibody formats is the single-chain variable (V)-domain antibody fragment (scFv), which comprises V domains from the heavy and light chain (VH and VL domain) linked by a peptide linker of ˜15 amino acid residues. A peptibody or peptide-Fc fusion is yet another antibody protein product. The structure of a peptibody consists of a biologically active peptide grafted onto an Fc domain. Peptibodies are well-described in the art. See, e.g., Shimamoto et al., mAbs 4 (5): 586-591 (2012). Other antibody protein products include a single chain antibody (SCA); a diabody; a triabody; a tetrabody, and the like.

In various aspects, the antigen-binding protein of the present disclosure comprises, consists essentially of, or consists of any one of these antibody protein products. In various aspects, the antigen-binding protein of the present disclosure comprises, consists essentially of, or consists of any one of an scFv, Fab′, F(ab′)2, VHH/VH, Fv fragment, ds-scFv, scFab′, half antibody-scFv, heterodimeric Fab/scFv-Fc, heterodimeric scFv-Fc, heterodimeric IgG (CrossMab), tandem scFv, tandem biparatopic scFv, Fab/scFv-Fc, tandem Fab′, single-chain diabody, dimeric antibody, multimeric antibody (e.g., a diabody, triabody, tetrabody), miniAb, peptibody VHH/VH of camelid heavy chain antibody, sdAb, diabody (single-chain diabody, homodimeric diabody, heterodimeric diabody, tandem diabody (TandAb), diabody that self-dimerizes), a triabody, a tetrabody. An ordinarily skilled artisan would understand that any bispecific antigen-binding protein formats can be used to generate biparatopic antigen-binding protein formats. In some embodiments, the antigen-binding protein is a dual-affinity re-targeting antibody (DART). In some embodiments, the antigen-binding protein is a bispecific T-cell engager (BiTE).

In various aspects, the antigen-binding protein of the present disclosure is linked to an agent. As described below, the agent may be any known in the art, including, but not limited to, chemotherapeutic agents, cytokines and growth factors, cytotoxic agents, detectable agent (e.g., fluorescein), and the like.

The antigen-binding proteins provided herein bind to CTLA4 in a non-covalent and reversible manner. In various embodiments, the binding strength of the antigen-binding protein to CTLA4 may be described in terms of its affinity, a measure of the strength of interaction between the binding site of the antigen-binding protein and the epitope. In various aspects, the antigen-binding proteins provided herein have high-affinity for CTLA4 and thus will bind a greater amount of CTLA4 in a shorter period of time than low-affinity antigen-binding proteins. In various aspects, the antigen-binding protein has an equilibrium association constant, K_{A}, which is at least 105 mol⁻¹, at least 10⁶ mol⁻¹, at least 107 mol⁻¹, at least 108 mol⁻¹, at least 109 mol⁻¹, or at least 1010 mol⁻¹. As understood by the artisan of ordinary skill, K_{A} can be influenced by factors including pH, temperature and buffer composition.

In various embodiments, the binding strength of the antigen-binding protein to CTLA4 may be described in terms of its sensitivity. K_{D} is the equilibrium dissociation constant, a ratio of k_{off}/kₒₙ, between the antigen-binding protein and CTLA4. K_{D} and K_{A} are inversely related. The K_{D} value relates to the concentration of the antigen-binding protein (the amount of antigen-binding protein needed for a particular experiment) and so the lower the K_{D} value (lower concentration) the higher the affinity of the antigen-binding protein. In various aspects, the binding strength of the antigen-binding protein to CTLA4 may be described in terms of K_{D}. In various aspects, the K_{D} of the antigen-binding proteins provided herein is about 10⁻¹, about 10⁻², about 10⁻³, about 10⁻⁴, about 10⁻⁵, about 10⁻⁶, or less. In various aspects, the K_{D} of the antigen-binding proteins provided herein is micromolar, nanomolar, picomolar or femtomolar. In various aspects, the K_{D} of the antigen-binding proteins provided herein is within a range of about 10⁻⁴ to 10⁻⁶ or 10⁻⁷ to 10⁻⁹ or 10⁻¹⁰ to 10⁻¹² or 10⁻¹³ to 10⁻¹⁵. In various aspects, the K_{D} of the antigen-binding proteins provided herein is within a range of about 1.0×10⁻¹² M to about 1.0×10⁻⁸ M. In various aspects, the K_{D} of the antigen-binding proteins is within a range of about 1.0×10⁻¹¹ M to about 1.0×10⁻⁹ M.

In various aspects, the affinity of the antigen-binding proteins are measured or ranked using a flow cytometry- or Fluorescence-Activated Cell Sorting (FACS)-based assay. Flow cytometry-based binding assays are known in the art. See, e.g., Cedeno-Arias et al., Sci Pharm 79 (3): 569-581 (2011); Rathanaswami et al., Analytical Biochem 373:52-60 (2008); and Geuijen et al., J Immunol Methods 302 (1-2): 68-77 (2005). In various aspects, the affinity of the antigen-binding proteins are measured or ranked using a competition assay as described in Trikha et al., Int J Cancer 110:326-335 (2004) and Tam et al., Circulation 98 (11): 1085-1091 (1998), as well as below.

Avidity gives a measure of the overall strength of an antigen-binding protein-antigen complex. It is dependent on three major parameters: affinity of the antigen-binding protein for the epitope, valency of both the antigen-binding protein and CTLA4, and structural arrangement of the parts that interact. The greater an antigen-binding protein's valency (number of antigen binding sites), the greater the amount of antigen (CTLA4) it can bind. In various aspects, the antigen-binding proteins have a strong avidity for CTLA4. In various aspects, the antigen-binding proteins are multivalent. In various aspects, the antigen-binding proteins are bivalent. In various instances, the antigen antigen-binding proteins are monovalent.

In certain aspect, presented herein is an engineered antigen-binding protein that specifically binds to multiple epitopes on CTLA4.

In some embodiments, the engineered antigen-binding protein specifically binds Epitope 1 defined by the residues ¹³⁴MYPPPY¹³⁹ (SEQ ID NO: 1) and Epitope 2 defined by the residues ⁶⁵SICT⁶⁸ (SEQ ID NO: 2) of CTLA4.

In some embodiments, the engineered antigen-binding protein comprises: a) a heavy chain variable domain (VH) sequence with at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identity to a VH sequence selected from the group consisting of the VH sequences listed in Table 3; and/or b) a light chain variable domain (VL) sequence with at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identity to a VL sequence selected from the group consisting of the VL sequences listed in Table 3.

In some embodiments, the engineered antigen-binding protein comprises a sequence with at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identity to the sequence selected from SEQ ID NOs: 9, 10, 12, 14, 15, 17, 19, 20-24, 27, 28, and 29-54.

In some embodiments, the engineered antigen-binding protein comprises a sequence with at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identity to the sequence selected from: a) SEQ ID NOs: 9 and 10; b) SEQ ID NOs: 9 and 14; c) SEQ ID NOs: 9 and 17; d) SEQ ID NOs: 12 and 10; e) SEQ ID NOs: 12 and 14; f) SEQ ID NOs: 12 and 17; g) SEQ ID NOs: 15 and 10; h) SEQ ID NOs: 15 and 14; i) SEQ ID NOs: 15 and 17; j) SEQ ID NOs: 9, 10, 15, and 17; k) SEQ ID NOs: 12, 14, 15, and 17; 1) SEQ ID NOs: 21 and 22; m) SEQ ID NOs: 27 and 28; n) SEQ ID NOs: 31, 32, and 33; 0) SEQ ID NOs: 34, 35, and 36; p) SEQ ID NOs: 37, 38, and 39; q) SEQ ID NOs: 40, 41, and 42; r) SEQ ID NOs: 43 and 44; s) SEQ ID NOs: 45 and 46; t) SEQ ID NOs: 47, 48, and 49; u) SEQ ID NOs: 50, 51, and 52; and v) SEQ ID NOS: 53 and 54.

In some embodiments, the engineered antigen-binding protein comprises: a) a VH sequence selected from the group consisting of the VH sequences listed in Table 3; and/or b) a VL sequence selected from the group consisting of the VL sequences listed in Table 3.

In some embodiments, the engineered antigen-binding protein comprises a sequence selected from SEQ ID NOs: 9, 10, 12, 14, 15, 17, 19, 20-24, 27, 28, and 29-54.

In some embodiments, the engineered antigen-binding protein comprises a sequence selected from: a) SEQ ID NOs: 9 and 10; b) SEQ ID NOs: 9 and 14; c) SEQ ID NOs: 9 and 17; d) SEQ ID NOs: 12 and 10; e) SEQ ID NOs: 12 and 14; f) SEQ ID NOs: 12 and 17; g) SEQ ID NOs: 15 and 10; h) SEQ ID NOs: 15 and 14; i) SEQ ID NOs: 15 and 17; j) SEQ ID NOs: 9, 10, 15, and 17; k) SEQ ID NOs: 12, 14, 15, and 17; 1) SEQ ID NOs: 21 and 22; m) SEQ ID NOs: 27 and 28; n) SEQ ID NOs: 31, 32, and 33; o) SEQ ID NOs: 34, 35, and 36; p) SEQ ID NOs: 37, 38, and 39; q) SEQ ID NOs: 40, 41, and 42; r) SEQ ID NOs: 43 and 44; s) SEQ ID NOs: 45 and 46; t) SEQ ID NOs: 47, 48, and 49; u) SEQ ID NOs: 50, 51, and 52; and v) SEQ ID NOs: 53 and 54.

In some embodiments, the engineered antigen-binding protein is selected from an antibody, Fv, F(ab′)2, Fab′, dsFv, scFv, sc(Fv)2, half antibody-scFv, tandem scFv, tandem biparatopic scFv, Fab/scFv-Fc, tandem Fab′, single-chain diabody, tandem diabody (TandAb), Fab/scFv-Fc, heterodimeric Fab/scFv-Fc, heterodimeric scFv-Fc, heterodimeric IgG (CrossMab), DART, and diabody.

In certain embodiments, the engineered antigen-binding protein comprises an immunoglobulin heavy chain constant domain selected from the group consisting of IgG, IgG1, IgG2, IgG2A, IgG2B, IgG3, IgG4, IgA, IgM, IgD, and IgE constant domains.

In some embodiments, the engineered antigen-binding protein comprises an Fc domain. In some embodiments, the Fc domain is a functional or wild-type Fc domain that can bind to one or more Fc receptors. In some embodiments, the Fc domain may be nonfunctional, e.g., comprise a mutation, deletion, substitution, addition of one or more critical amino acids such that Fc domain (while structurally present) can no longer bind to one or more Fc receptors. In other embodiments, the engineered antigen-binding protein does not comprise (a) an Fc domain, or (b) the CH2 domain and/or CH3 domain of the constant region of an antibody. Accordingly, in some embodiments, the engineered antigen-binding protein does not bind to one or more Fc receptors, irrespective of whether the engineered antigen-binding protein comprises the Fc domain.

In certain aspects, provided herein is an engineered antigen-binding protein that specifically binds to CTLA4 and lacks the CH2 domain and/or a CH3 domain of the constant region of an antibody.

In some embodiments, the engineered antigen-binding protein comprises: a) a heavy chain variable domain (VH) sequence with at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identity to a VH sequence selected from the group consisting of the VH sequences listed in Table 3; and/or b) a light chain variable domain (VL) sequence with at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identity to a VL sequence selected from the group consisting of the VL sequences listed in Table 3.

In some embodiments, the engineered antigen-binding protein comprises a sequence with at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identity to the sequence selected from SEQ ID NOs: 9, 10, 12, 14, 15, 17, 19, 20-24, 27, 28, and 29-54.

In some embodiments, the engineered antigen-binding protein comprises a sequence with at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identity to the sequence selected from: a) SEQ ID NOs: 9 and 10; b) SEQ ID NOs: 9 and 14; c) SEQ ID NOs: 9 and 17; d) SEQ ID NOs: 12 and 10; e) SEQ ID NOs: 12 and 14; f) SEQ ID NOs: 12 and 17; g) SEQ ID NOs: 15 and 10; h) SEQ ID NOs: 15 and 14; i) SEQ ID NOs: 15 and 17; j) SEQ ID NOs: 9, 10, 15, and 17; k) SEQ ID NOs: 12, 14, 15, and 17; 1) SEQ ID NOs: 21 and 22; m) SEQ ID NOs: 27 and 28; n) SEQ ID NOs: 31, 32, and 33; o) SEQ ID NOs: 34, 35, and 36; p) SEQ ID NOs: 37, 38, and 39; q) SEQ ID NOs: 40, 41, and 42; r) SEQ ID NOs: 43 and 44; s) SEQ ID NOs: 45 and 46; t) SEQ ID NOs: 47, 48, and 49; u) SEQ ID NOs: 50, 51, and 52; and v) SEQ ID NOS: 53 and 54.

In some embodiments, the engineered antigen-binding protein comprises: a) a VH sequence selected from the group consisting of the VH sequences listed in Table 3; and/or b) a VL sequence selected from the group consisting of the VL sequences listed in Table 3.

In some embodiments, the engineered antigen-binding protein comprises a sequence selected from SEQ ID NOs: 9, 10, 12, 14, 15, 17, 19, 20-24, 27, 28, and 29-40.

In some embodiments, the engineered antigen-binding protein comprises a sequence selected from: a) SEQ ID NOs: 9 and 10; b) SEQ ID NOs: 9 and 14; c) SEQ ID NOs: 9 and 17; d) SEQ ID NOs: 12 and 10; e) SEQ ID NOs: 12 and 14; f) SEQ ID NOs: 12 and 17; g) SEQ ID NOs: 15 and 10; h) SEQ ID NOs: 15 and 14; i) SEQ ID NOs: 15 and 17; j) SEQ ID NOs: 9, 10, 15, and 17; k) SEQ ID NOs: 12, 14, 15, and 17; 1) SEQ ID NOs: 21 and 22; m) SEQ ID NOs: 27 and 28; n) SEQ ID NOs: 31, 32, and 33; 0) SEQ ID NOs: 34, 35, and 36; p) SEQ ID NOs: 37, 38, and 39; q) SEQ ID NOs: 40, 41, and 42; r) SEQ ID NOs: 43 and 44; s) SEQ ID NOs: 45 and 46; t) SEQ ID NOs: 47, 48, and 49; u) SEQ ID NOs: 50, 51, and 52; and v) SEQ ID NOs: 53 and 54.

In some embodiments, the engineered antigen-binding protein is selected from Fv, F(ab′)2, Fab′, dsFv, scFv, sc(Fv)2, half antibody-scFv, tandem scFv, tandem biparatopic scFv, Fab/scFv-Fc, tandem Fab′, single-chain diabody, tandem diabody (TandAb), Fab/scFv-Fc, heterodimeric Fab/scFv-Fc, heterodimeric scFv-Fc, heterodimeric IgG (CrossMab), DART, and diabody.

In certain embodiments, the engineered antigen-binding protein is F(ab′)2. In some embodiments, the engineered antigen-binding protein comprises the sequence of SEQ ID NOs: 27 and 28. In some embodiments, the engineered antigen-binding protein comprises the sequence of SEQ ID NOs: 21 and 22.

In certain embodiments, the engineered antigen-binding protein binds two epitopes: Epitope 1 defined by the residues ¹³⁴MYPPPY¹³⁹ (SEQ ID NO: 1) and Epitope 2 defined by the residues ⁶⁵SICT⁶⁸ (SEQ ID NO: 2) of CTLA4.

In some embodiments, the engineered antigen-binding protein comprises at least two different VH domains and at least two different VL domains.

In some embodiments, the engineered antigen-binding protein is a DART or a diabody. In some embodiments, the diabody is a homodimeric diabody or a heterodimeric diabody. In some embodiments, the diabody is a single-chain diabody or a tandem diabody (TandAb). In some embodiments, the diabody is a tandem diabody.

In certain embodiments, the DART or the diabody comprises, in the N-terminal to C-terminal direction: a) a first VH; b) a first VL; c) a second VH; and d) a second VL; wherein the first VH and the first VL are linked by a Linker A; the first VL and the second VH are linked by a Linker B; and the second VH and the second VL are linked by a Linker C.

In some embodiments, the Linker A and/or Linker B comprise a peptide sequence of (GlyGlySer)n, wherein n=1-20 (SEQ ID NO: 74). In some embodiments, the n=0, 1, 2, or 3. In some embodiments, the n=3.

In some embodiments, the Linker C comprises a peptide sequence of (GlyGlySer)n, wherein n=1-20 (SEQ ID NO: 74). In some embodiments, the n is greater than 4. In some embodiments, the n=0, 1, 2, 3, or 4. In some embodiments, the n=3.

In some embodiments, each of the Linker A, Linker B, and Linker C comprises (GlyGlySer)n, wherein n=3 (SEQ ID NO: 75).

In certain embodiments, the first VH and the second VL bind the Epitope 1 (SEQ ID NO: 1); and the first VL and second VH bind the Epitope 2 (SEQ ID NO: 2). In some embodiments, the first VH, first VL, second VH, second VL comprise the sequence with at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identity to SEQ ID NO: 15, SEQ ID NO: 14, SEQ ID NO: 12, and SEQ ID NO: 17, respectively. In some embodiments, the first VH, first VL, second VH, second VL comprise the sequence of SEQ ID NO: 15, SEQ ID NO: 14, SEQ ID NO: 12, and SEQ ID NO: 17, respectively.

In some embodiments, the engineered antigen-binding protein comprises the sequence with at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identity to SEQ ID NO: 24 (BioE2052). In some embodiments, the engineered antigen-binding protein comprises the sequence of SEQ ID NO: 24 (BioE2052).

In certain embodiments, the first VH and the second VL bind the Epitope 2 (SEQ ID NO: 2); and the first VL and second VH bind the Epitope 1 (SEQ ID NO: 1). In some embodiments, the first VH, first VL, second VH, second VL comprise the sequence with at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identity to SEQ ID NO: 12, SEQ ID NO: 17, SEQ ID NO: 15, and SEQ ID NO: 14, respectively. In some embodiments, the first VH, first VL, second VH, second VL comprise the sequence as set forth in SEQ ID NO: 12, SEQ ID NO: 17, SEQ ID NO: 15, and SEQ ID NO: 14, respectively.

In some embodiments, the engineered antigen-binding protein comprises the sequence with at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identity to SEQ ID NO: 23 (BioE2051). In some embodiments, the engineered antigen-binding protein comprises the sequence of SEQ ID NO: 23 (BioE2051).

Numerous embodiments are further provided that can be applied to any aspect of the present invention and/or combined with any other embodiment described herein. For example, in some embodiments, the CTLA4 is human CTLA4. In some embodiments, the engineered antigen-binding protein is chimeric, humanized, composite, murine, or human. In some embodiments, the engineered antigen-binding protein further comprises a peptide tag (e.g., Histidine tag). In some embodiments, the engineered antigen-binding protein further comprises a leader sequence. In some embodiments, the leader sequence comprises the sequence set forth in SEQ ID NO: 55. In some embodiments, the engineered antigen-binding protein is conjugated or detectably labeled, optionally wherein the engineered antigen-binding protein is PEGylated, e.g., to increase the half-life in circulation or for better solubility. In some embodiments, the engineered antigen-binding protein self-dimerizes. In some embodiments, the engineered antigen-binding protein: a) blocks the interaction between CTLA4 and its ligands (e.g., CD80 (B7-1) and CD86 (B7-2)); and/or b) increases the interleuken-2 (IL-2) expression by the T cells.

In certain aspects, provided herein is an isolated nucleic acid molecule that encodes the engineered antigen-binding protein of the present disclosure. Also provided herein is a vector comprising such isolated nucleic acid. Further provided herein is a host cell which comprises the said isolated nucleic acid, comprises the said vector, or expresses the engineered antigen-binding protein of the present disclosure.

In certain aspects, provided herein is a pharmaceutical composition comprising the engineered antigen-binding protein of the present disclosure, an isolated nucleic acid that encodes said engineered antigen-binding protein, a vector comprising said isolated nucleic acid, or a host cell comprising the said isolated nucleic acid, comprises the said vector, or expresses the engineered antigen-binding protein of the present disclosure.

In certain aspects, provided herein is a kit comprising at least one engineered antigen-binding protein of the present disclosure.

### Sequence Identity/Homology

Function-conservative variants are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A function-conservative variant also includes a polypeptide which has at least 60% amino acid identity as determined by BLAST or FASTA algorithms, preferably at least 75%, more preferably at least 85%, still preferably at least 90%, and even more preferably at least 95%, and which has the same or substantially similar properties or functions as the native or parent protein to which it is compared.

The percent identity between two sequences is a function of the number of identical positions shared by the sequences (i.e., % identity=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available on the World Wide Web at the GCG company website), using a NWSgapdna. CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:11 17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48): 444 453 (1970)) algorithm which has been incorporated into the GAP program in the GCG software package (available on the world wide web at the GCG company website), using either a Blosum 62 matrix or a PAM250 matrix, and a gap weight of 16, 14, 12, 10, 8, 6, or 4 and a length weight of 1, 2, 3, 4, 5, or 6.

The nucleic acid and protein sequences of the present invention can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403 10. BLAST nucleotide searches can be performed with the NBLAST program, score=100, wordlength=12 to obtain nucleotide sequences homologous to the nucleic acid molecules of the present invention. BLAST protein searches can be performed with the XBLAST program, score=50, wordlength=3 to obtain amino acid sequences homologous to the protein molecules of the present invention. To obtain gapped alignments for comparison purposes, Gapped BLAST can be utilized as described in Altschul et al., (1997) Nucleic Acids Res. 25 (17): 3389 3402. When utilizing BLAST and Gapped BLAST programs, the default parameters of the respective programs (e.g., XBLAST and NBLAST) can be used (available on the World Wide Web at the NCBI website).

### Methods of Antibody Production and Related Methods

Suitable methods of making antigen-binding proteins (e.g., antibodies, antigen-binding antibody fragments, and antibody protein products) are known in the art. For instance, standard hybridoma methods for producing antibodies are described in, e.g., Harlow and Lane (eds.), Antibodies: A Laboratory Manual, CSH Press (1988), and CA. Janeway et al. (eds.), Immunobiology, 5^{th} Ed., Garland Publishing, New York, NY (2001)).

Depending on the host species, various adjuvants can be used to increase the immunological response leading to greater antibody production by the host. Such adjuvants include but are not limited to Freund's, mineral gels such as aluminum hydroxide, and surface active substances such as lysolecithin, pluronic polyols, polyanions, peptides, oil emulsions, keyhole limpet hemocyanin, and dinitrophenol. BCG (bacilli Calmette-Guerin) and Corynebacterium parvum are potentially useful human adjuvants.

Other methods of antibody production are summarized in Table 2.

TABLE 2
Alternative Methods of Antibody Production

| Technique | Various references |
|---|---|
| EBV-hybridoma methods and | Haskard and Archer, J. Immunol. Methods, |
| Bacteriophage vector | 74(2), 361-67 (1984), Roder et al., Methods |
| expression systems | Enzymol., 121, 140-67 (1986), and Huse et al., |
| | Science, 246, 1275-81 (1989)). |
| methods of producing antibodies | U.S. Pat. Nos. 5,545,806, 5,569,825, and |
| in non-human animals | 5,714,352, and U.S. Patent Application |
| | Publication No. 2002/0197266 |
| inducing in vivo production in | Orlandi et al. (Proc Natl Acad Sci 86: 3833- |
| the lymphocyte population or | 3837; 1989), and Winter G and Milstein C |
| by screening recombinant | (Nature 349: 293-299, 1991). |
| immunoglobulin libraries or | |
| panels of highly specific | |
| binding reagents | |
| methods of producing | Protein production and purification” Nat |
| recombinant proteins | Methods 5(2): 135-146 (2008). |
| Phage display | Janeway et al., supra, Huse et al., supra, and |
| | U.S. Pat. No. 6,265,150). Related methods also |
| | are described in U.S. Pat. No. 5,403,484; U.S. |
| | Pat. No. 5,571,698; U.S. Pat. No. |
| | 5,837,500; U.S. Pat. No. 5,702,892. The |
| | techniques described in U.S. Pat. No. |
| | 5,780,279; U.S. Pat. No. 5,821,047; U.S. |
| | Pat. No. 5,824,520; U.S. Pat. No. |
| | 5,855,885; U.S. Pat. No. 5,858,657; U.S. |
| | Pat. No. 5,871,907; U.S. Pat. No. |
| | 5,969,108; U.S. Pat. No. 6,057,098; and U.S. |
| | Pat. No. 6,225,447 |
| Antibodies can be produced | U.S. Pat. Nos. 5,545,806 and 5,569,825, and |
| by transgenic mice | Janeway et al., supra. |

An antigen-binding protein can be engineered to increase or improve its pharmacokinetic (PK) properties (e.g., half-life). Numerous properties of an antigen-binding protein can influence pharmacokinetics including, but not limited to, molecular size, folding stability, solubility, target interaction, neonatal Fc binding capacity, and charge. Modifications to the antigen-binding protein include, but are not limited to antigen-binding domain conjugation to one or more carrier proteins, PEGylation, acylation (e.g., by conjugation to a fatty acid molecule), polysialylation, or glycosylation. Amino acid sequence modifications can be used to improve or optimize the PK properties of the protein, and conjugation to large, slowly metabolized macromolecules can also modify the PK properties of the protein. Macromolecules that can be conjugated to the antigen protein include, but are not limited to, proteins (e.g., albumin), polysaccharides (e.g., sepharose, agarose, cellulose, or cellulose beads), polymeric amino acids (polyglutamic acid or polylysine), amino acid copolymers, inactivated virus particles, inactivated bacterial toxins (e.g., leukotoxin or diphtheria, tetanus, or cholera toxins or molecules), inactivated bacteria, dendritic cells, thyroglobulin, polyamino acids (e.g., poly(D-lysine: D-glutamic acid)), VP6 polypeptides of rotaviruses, influenza virus hemaglutinin, influenza virus nucleoprotein, Keyhole Limpet Hemocyanin (KLH), and hepatitis B virus core protein and surface antigen (WO2021146436). Additional PK modulators known in the art include lipophiles, bile acids, steroids, phospholipid analogues, and vitamins, examples of which include, but are not limited to, cholesterol, fatty acids, cholic acid, lithocholic acid, dialkylglycerides, diacylglyceride, phospholipids, sphingolipids, naproxen, ibuprofen, vitamin E, and biotin (U.S. Pat. No. 9,322,018). Methods for producing modified antigen-binding proteins as described herein are known in the art.

In some embodiments, the antigen-binding protein receptor is fused or otherwise linked to a conventional fragment crystallizable region (Fc Region). For example, the Fc region can be an IgG1, IgG2, IgG3, or IgG4 Fc region. In some embodiments, mutations in the Fc region of the antigen-binding protein can be engineered to modulate its interaction with the neonatal Fc receptor (FcRn), which is involved in receptor-mediated internalization and recycling of IgG occur via FcRn (Sockolosky and Szoka, Adv Drug Deliv Rev. (2015) 109-24), thereby improving its pharmacokinetic properties (US20210277092). For example, the Fc region can comprise a LALAPG mutation that inhibits binding of the antigen-binding protein to the neonatal Fcγ receptor. In some embodiments, the antigen-binding protein is fused or otherwise linked to an albumin-binding protein.

In some embodiments, the macromolecule is directly conjugated to the antigen-binding protein. In some embodiments, the macromolecule is fused to the antigen-binding peptide via a linker.

Modified antigen-binding proteins as described herein can have improved or optimized pharmacokinetic (PK) properties, for example, a plasma half-life in a human subject of greater than 4 hours, 5 hours, 6 hours, 7 hours, 8 hours, 9 hours, 10 hours, 12 hours, 18 hours, 24 hours, 2 days, 3 days, 4 days, 5 days, 6 days, 7 days, 10 days, 14 days, or 30 days.

Methods of testing the antigen-binding protein for the ability to bind to the epitope(s) of CTLA4 regardless of how the antigen-binding proteins are produced are known in the art and include any binding assay, such as, for example, radioimmunoassay (RIA), ELISA, Western blot, immunoprecipitation, SPR, and competitive inhibition assays (see, e.g., U.S. Patent Application Publication No. 2002/0197266).

### Sequences

As used herein, coding region refers to regions of a nucleotide sequence comprising codons which are translated into amino acid residues, whereas noncoding region refers to regions of a nucleotide sequence that are not translated into amino acids (e.g., 5′ and 3′ untranslated regions).

Complement [to] or complementary refers to the broad concept of sequence complementarity between regions of two nucleic acid strands or between two regions of the same nucleic acid strand. It is known that an adenine residue of a first nucleic acid region is capable of forming specific hydrogen bonds (base pairing) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, at least or about 50%, and preferably at least or about 75%, at least or about 90%, or at least or about 95% of the nucleotide residues of the first portion are capable of base pairing with nucleotide residues in the second portion. In other embodiments, all nucleotide residues of the first portion are capable of base pairing with nucleotide residues in the second portion.

A nucleic acid is operably linked when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In making the changes in the amino sequences of polypeptide, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamate (−3.5); glutamine (−3.5); aspartate (<RTI 3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well-known to those of skill in the art and include: arginine and lysine; glutamate and aspartate; serine and threonine; glutamine and asparagine; and valine, leucine and isoleucine.

In view of the foregoing, the nucleotide sequence of a DNA or RNA can be used to derive the polypeptide amino acid sequence, using the genetic code to translate the DNA or RNA into an amino acid sequence. Likewise, for polypeptide amino acid sequence, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

TABLE 3
Sequence of representative CTLA4-binding proteins or fragments thereof

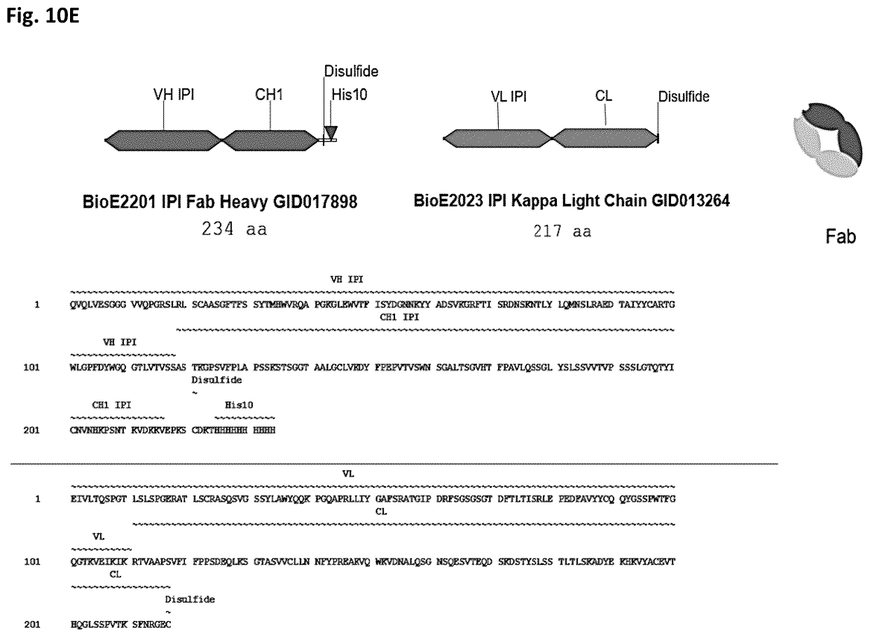

```
SEQ ID NO: 9
Heavy chain variable domain (VH) of the 26 antibody-amino acid
sequence
      1 MDVLVLFLCL VAFPSCVLSQ VQLKESGPGL VAPSQSLSIT CTVSGFSLTS YGVYWVRQPP
    61 GKGLEWLGVI WAGGTTNYNS ALMSRLSISK DNSKSQVFLK MSSLQTDDTA MYYCARGPPH
  121 AMMKRGYAMD YWGQGTSVIV SS
SEQ ID NO: 10
Light chain variable domain (VL) of the 26 antibody-amino acid
      1 MDFQVQIFSF LLISASVILS RGQNVLTQSP AIMPASPGEK VTMTCSATSS ITYMSWYQQK
    61 SGSSPRLLIY DTSNLASGVP VRESGSGSGT SYSLTISRME AEDAATYYCQ QWSSYPLTFG
  121 AGTKLELK
SEQ ID NO: 11
Light chain variable domain (VL) of the 26 antibody-cDNA
sequence
      1 atggattttc aagtgcagat tttcagcttc ctgctaatca gtgcctcagt catactgtcc
    61 agaggacaaa atgttctcac ccagtctcca gcaatcatgc ctgcatctcc aggggagaag
  121 gtcaccatga cctgcagtgc cacctcaagt ataacttaca tgtcctggta ccagcagaag
  181 tcaggatcct cccccagact cctgatttat gacacatcca acctggcttc tggagtccct
  241 gttcgcttca gtggcagtgg gtctgggacc tcttactctc tcacaatcag ccgaatggag
  301 gctgaagatg ctgccactta ttactgccag cagtggagta gttacccgct cacgttcggt
  361 gctgggacca agctggagct gaaa
SEQ ID NO: 12
Heavy chain variable domain (VH) of the 121 antibody-amino
acid sequence
      1 QVQLQESGPG LVKPSQTLSL TCTVSGFSLT SYGVYWVRQP PGKGLEWLGV IWAGGTTNYN
    61 SALMSRLTIS KDTSKNQVSL KLSSVTAADT AVYYCARGPP HAMMKRGYAM DYWGQGTLVT
  121 VSS
SEQ ID NO: 13
Heavy chain variable domain (VH) of the 121 antibody-cDNA
      1 caggtgcagc tgcaagagtc aggacctggc ctggtgaagc cctcacagac actgtccttg
    61 acttgcactg tctctgggtt ttcattaacc tcatatggtg tatattgggt tcgccagcct
  121 ccaggaaagg gtctggagtg gctgggagta atatgggctg gtggtaccac aaattataat
  181 tcggctctca tgtccagact gacaatcagc aaagacacat ccaagaacca agtttcctta
  241 aaactcagca gtgtgactgc agcggacaca gccgtctact actgtgcccg aggccccccg
  301 cacgctatga tgaagagagg ctatgctatg gactactggg gacaaggaac cctagtcaca
  361 gtctcctcag g
SEQ ID NO: 14
Light chain variable domain (VL) of the 121 antibody-amino acid
sequence
      1 DIQMTQSPSS LSASVGDRVT ITCSATSSIT YMSWYQQKPG KAPKLLIYDT SNLASGVPSR
    61 FSGSGSGTDY TLTISSLOPE DFATYYCQQW SSYPLTFGGG TKLEIK
SEQ ID NO: 15
Heavy chain variable domain (VH) of ipilimumab-amino acid
sequence
      1 QVQLVESGGG VVQPGRSLRL SCAASGFTFS SYTMHWVRQA PGKGLEWVTF ISYDGNNKYY
    61 ADSVKGRFTI SRDNSKNTLY LQMNSLRAED TAIYYCARTG WLGPFDYWGQ GTLVTVSS
SEQ ID NO: 16
Heavy chain variable domain (VH) of ipilimumab-cDNA
sequence
      1 caggtgcagc tggtggagtc tgggggaggc gtggtccagc ctgggaggtc cctgagactc
    61 tcctgtgcag cctctggatt caccttcagt agctatacta tgcactgggt ccgccaggct
  121 ccaggcaagg ggctggagtg ggtgacattt atatcatatg atggaaacaa taaatactac
  181 gcagactccg tgaagggccg attcaccatc tccagagaca attccaagaa cacgctgtat
  241 ctgcaaatga acagcctgag agctgaggac acggctatat attactgtgc gaggaccggc
  301 tggctggggc cctttgacta ctggggccag ggaaccctgg tcaccgtctc ctcag
SEQ ID NO: 17
Light chain variable domain (VL) of ipilimumab-amino acid
sequence
      1 EIVLTQSPGT LSLSPGERAT LSCRASQSVG SSYLAWYQQK PGQAPRLLIY GAFSRATGIP
    61 DRESGSGSGT DETLTISRLE PEDFAVYYCQ QYGSSPWTFG QGTKVEIK
SEQ ID NO: 18 Light chain variable domain (VL) of ipilimumab-cDNA
sequence
      1 gaaattgtgt tgacgcagtc tccaggcacc ctgtctttgt ctccagggga aagagccacc
    61 ctctcctgca gggccagtca gagtgttggc agcagctact tagcctggta ccagcagaaa
  121 cctggccagg ctcccaggct cctcatctat ggtgcattca gcagggccac tggcatccca
  181 gacaggttca gtggcagtgg gtctgggaca gacttcactc tcaccatcag cagactggag
  241 cctgaagatt ttgcagtgta ttactgtcag cagtatggta gctcaccgtg gacgttcggc
  301 caagggacca aggtggaaat caaac
SEQ ID NO: 19
BioE2021 (ipilimumab ScFv) amino acid sequence
      1 EIVLTQSPGT LSLSPGERAT LSCRASQSVG SSYLAWYQQK PGQAPRLLIY GAFSRATGIP
    61 DRFSGSGSGT DFTLTISRLE PEDFAVYYCQ QYGSSPWTFG QGTKVEIKGG GGSGGGGSGG
  121 GGSQVQLVES GGGVVQPGRS LRLSCAASGF TFSSYTMHWV RQAPGKGLEW VTFISYDGNN
  181 KYYADSVKGR FTISRDNSKN TLYLQMNSLR AEDTAIYYCA RTGWLGPFDY WGQGTLVTVS
  241 S
SEQ ID NO: 20
BioE2031 (121 ScFv) amino acid sequence
      1 DIQMTQSPSS LSASVGDRVT ITCSATSSIT YMSWYQQKPG KAPKLLIYDT SNLASGVPSR
    61 FSGSGSGTDY TLTISSLQPE DFATYYCQQW SSYPLTFGGG TKVEIKGGGG SGGGGSGGGG
  121 SQVQLQESGP GLVKPSQTLS LTCTVSGFSL TSYGVYWVRQ PPGKGLEWLG VIWAGGTTNY
  181 NSALMSRLTI SKDTSKNQVS LKLSSVTAAD TAVYYCARGP PHAMMKRGYA MDYWGQGTLV
  241 TVSS
SEQ ID NO: 21
121 VH and CH1 amino acid sequence
      1 QVQLQESGPG LVKPSQTLSL TCTVSGFSLT SYGVYWVROP PGKGLEWLGV IWAGGTTNYN
    61 SALMSRLTIS KDTSKNQVSL KLSSVTAADT AVYYCARGPP HAMMKRGYAM DYWGQGTLVT
  121 VSSASTKGPS VFPLAPSSKS TSGGTAALGC LVKDYFPEPV TVSWNSGALT SGVHTFPAVL
  181 QSSGLYSLSS VVTVPSSSLG TQTYICNVNH KPSNTKVDKK V
*SEQ ID NO: 21 optionally comprises the sequence (EPKSCDKT) (SEQ ID NO: 76)
at the C-terminal end, which corresponds to the hinge region for F(ab′)2 or
Fab′, wthe cysteine residue forms inter-chain bond with kappa light.
Alternatively, F(ab′)2 may comprise the sequence (e.g., proprietary sequence
as indicated in FIG. 101) that forms inter-chain
dimerization domain C-terminal to SEQ ID NO: 21.
*See Table 3 and FIGS. 10F and 10I.
SEQ ID NO: 22
BioE2034 121 VL and CL amino acid sequence
      1 DIQMTQSPSS LSASVGDRVT ITCSATSSIT YMSWYQQKPG KAPKLLIYDT SNLASGVPSR
    61 FSGSGSGTDY TLTISSLQPE DEATYYCQQW SSYPLTFGGG TKLEIKRTVA APSVFIFPPS
  121 DEQLKSGTAS VVCLLNNEYP REAKVQWKVD NALQSGNSQE SVTEQDSKDS TYSLSSTLTL
  181 SKADYEKHKV YACEVTHQGL SSPVTKSENR GEC
SEQ ID NO: 23
BioE2051 (anti-CTLA4 diabody => single-chain diabody; self-
dimerizes; tandem diabody (TandAb)) amino acid sequence
      1 QVQLQESGPG LVKPSQTLSL TCTVSGESLT SYGVYWVRQP PGKGLEWLGV IWAGGTTNYN
    61 SALMSRLTIS KDTSKNQVSL KLSSVTAADT AVYYCARGPP HAMMKRGYAM DYWGQGTLVT
  121 VSSGGSGGSG GSEIVLTQSP GTLSLSPGER ATLSCRASQS VGSSYLAWYQ QKPGQAPRLL
  181 IYGAFSRATG IPDRESGSGS GTDETLTISR LEPEDFAVYY CQQYGSSPWT FGQGTKVEIK
  241 GGSGGSGGSQ VQLVESGGGV VQPGRSLRLS CAASGFTFSS YTMHWVRQAP GKGLEWVTFI
  301 SYDGNNKYYA DSVKGRFTIS RDNSKNTLYL QMNSLRAEDT AIYYCARTGW LGPFDYWGQG
  361 TLVTVSSGGS GGSGGSDIQM TQSPSSLSAS VGDRVTITCS ATSSITYMSW YQQKPGKAPK
  421 LLIYDTSNLA SGVPSRESGS GSGTDYTLTI SSLQPEDFAT YYCQQWSSYP LTFGGGTKVE
  481 IK
SEQ ID NO: 24
BioE2052 (anti-CTLA4 diabody => single-chain diabody; self-
dimerizes; tandem diabody (TandAb)) amino acid sequence
      1 QVQLVESGGG VVQPGRSLRL SCAASGETFS SYTMHWVRQA PGKGLEWVTF ISYDGNNKYY
    61 ADSVKGRFTI SRDNSKNTLY LQMNSLRAED TAIYYCARTG WLGPFDYWGQ GTLVTVSSGG
  121 SGGSGGSDIQ MTQSPSSLSA SVGDRVTITC SATSSITYMS WYQQKPGKAP KLLIYDTSNL
  181 ASGVPSRFSG SGSGTDYTLT ISSLQPEDFA TYYCQQWSSY PLTFGGGTKV EIKGGSGGSG
  241 GSQVQLQESG PGLVKPSQTL SLTCTVSGES LTSYGVYWVR QPPGKGLEWL GVIWAGGTTN
  301 YNSALMSRLT ISKDTSKNQV SLKLSSVTAA DTAVYYCARG PPHAMMKRGY AMDYWGQGTL
  361 VTVSSGGSGG SGGSEIVLTQ SPGTLSLSPG ERATLSCRAS QSVGSSYLAW YQQKPGQAPR
  421 LLIYGAFSRA TGIPDRESGS GSGTDFTLTI SRLEPEDFAV YYCQQYGSSP WTFGQGTKVE
  481 IK
SEQ ID NO: 25
BioE2051 (anti-CTLA4 diabody => single-chain diabody; self-
dimerizes; tandem diabody (TandAb)) cDNA sequence
      1 CAAGTGCAGC TCCAAGAGTC CGGCCCCGGC CTCGTGAAAC CCAGCCAGAC ACTGTCTCTG
    61 ACATGCACCG TGAGCGGCTT TTCTCTGACC AGCTACGGAG TGTATTGGGT GAGACAACCC
  121 CCCGGCAAGG GACTGGAGTG GCTGGGAGTG ATTTGGGCCG GCGGCACCAC CAACTACAAT
  181 AGCGCCCTCA TGTCTAGACT CACCATCTCC AAGGACACCA GCAAGAACCA AGTGTCCCTC
  241 AAGCTGTCCA GCGTCACAGC TGCCGACACC GCCGTGTACT ATTGTGCTAG AGGCCCCCCC
  301 CATGCCATGA TGAAGAGAGG CTATGCCATG GATTACTGGG GCCAAGGCAC ACTGGTGACC
  361 GTCAGCTCCG GAGGAAGCGG CGGCAGCGGA GGCTCCGAAA TTGTGCTCAC CCAGAGCCCC
  421 GGCACACTGT CTCTGAGCCC CGGCGAAAGG GCCACACTGA GCTGCAGAGC CTCCCAATCC
  481 GTGGGCAGCA GCTATCTGGC TTGGTATCAG CAGAAACCCG GCCAAGCCCC TAGACTGCTG
  541 ATCTATGGAG CCTTTTCTAG AGCTACCGGC ATCCCCGACA GATTCTCCGG CAGCGGCAGC
  601 GGCACAGACT TTACACTGAC AATTTCTAGA CTGGAACCAG AGGATTTCGC CGTCTACTAC
  661 TGCCAGCAGT ATGGAAGCAG CCCTTGGACC TTTGGCCAAG GCACCAAGGT GGAGATCAAG
  721 GGAGGAAGCG GAGGCAGCGG AGGCAGCCAA GTGCAGCTCG TGGAAAGCGG AGGAGGCGTG
  781 GTGCAGCCCG GCAGATCCCT CAGACTGAGC TGCGCCGCCA GCGGCTTCAC CTTCAGCTCC
  841 TATACCATGC ACTGGGTGAG GCAAGCCCCC GGCAAAGGAC TGGAGTGGGT CACCTTCATC
  901 AGCTACGACG GCAACAACAA GTACTACGCC GACAGCGTGA AGGGAAGGTT CACCATCTCT
  961 AGAGACAACT CCAAGAACAC CCTCTACCTC CAGATGAACT CTCTGAGGGC CGAAGACACC
1021 GCCATCTACT ACTGCGCTAG AACCGGCTGG CTGGGACCCT TTGACTACTG GGGACAAGGC
1081 ACACTGGTCA CAGTGTCCTC CGGAGGAAGC GGAGGCTCCG GCGGCAGCGA TATCCAGATG
1141 ACCCAATCCC CTTCCTCTCT GAGCGCCTCC GTGGGAGATA GGGTCACCAT TACATGTAGC
1201 GCCACAAGCA GCATCACCTA CATGAGCTGG TACCAGCAGA AACCCGGAAA GGCCCCTAAG
1261 CTGCTCATCT ACGACACCTC CAATCTGGCC AGCGGCGTGC CTTCTAGATT TAGCGGCTCC
1321 GGAAGCGGCA CAGATTACAC ACTGACAATC AGCTCTCTGC AGCCAGAGGA CTTCGCCACC
1381 TACTACTGTC AGCAGTGGAG CAGCTACCCT CTGACCTTTG GCGGCGGCAC CAAGGTGGAA
1441 ATCAAACACC ACCATCACCA CCATCACCAC CATCAC
SEQ ID NO: 26
BioE2052 (anti-CTLA4 diabody => single-chain diabody; self-
dimerizes; tandem diabody (TandAb)) cDNA sequence
      1 CAAGTGCAGC TCGTGGAATC CGGCGGAGGA GTCGTGCAGC CCGGCAGAAG CCTCAGACTG
    61 AGCTGCGCCG CCAGCGGATT CACCTTCAGC AGCTACACCA TGCACTGGGT GAGGCAAGCC
  121 CCCGGCAAAG GACTGGAGTG GGTCACATTC ATCTCCTACG ATGGCAACAA CAAGTACTAC
  181 GCCGACAGCG TGAAGGGAAG GTTTACCATC TCTAGAGATA ACTCCAAGAA CACCCTCTAC
  241 CTCCAGATGA ACTCTCTGAG AGCTGAGGAC ACAGCCATCT ATTACTGCGC TAGAACCGGA
  301 TGGCTGGGCC CTTTCGACTA CTGGGGACAA GGCACACTGG TGACAGTGTC CTCCGGAGGC
  361 AGCGGAGGCA GCGGAGGCAG CGACATCCAG ATGACCCAAT CCCCTAGCTC TCTGAGCGCC
  421 AGCGTGGGCG ATAGAGTGAC CATTACATGC TCCGCCACCA GCAGCATCAC CTACATGAGC
  481 TGGTACCAGC AAAAGCCCGG CAAAGCCCCC AAGCTGCTGA TCTACGATAC CAGCAATCTG
  541 GCCAGCGGCG TGCCTTCTAG ATTTTCCGGC TCCGGAAGCG GCACCGATTA CACACTGACC
  601 ATTTCCTCTC TGCAGCCAGA GGATTTCGCC ACCTACTATT GCCAGCAGTG GTCCTCCTAC
  661 CCTCTCACCT TTGGCGGCGG AACAAAGGTG GAGATCAAAG GCGGCAGCGG CGGCTCCGGA
  721 GGCAGCCAAG TCCAGCTGCA AGAGTCCGGA CCCGGACTGG TGAAACCTTC CCAGACACTG
  781 TCTCTGACAT GCACAGTGAG CGGATTCTCC CTCACAAGCT ACGGCGTGTA CTGGGTGAGA
  841 CAGCCTCCCG GCAAAGGACT GGAGTGGCTG GGCGTCATCT GGGCTGGAGG CACCACCAAT
  901 TACAACAGCG CTCTGATGTC TAGACTGACA ATCTCCAAGG ACACCAGCAA GAACCAAGTG
  961 TCTCTGAAGC TGAGCTCCGT GACAGCCGCC GATACAGCCG TGTACTATTG TGCCAGAGGC
1021 CCCCCCCACG CTATGATGAA GAGGGGCTAC GCCATGGACT ACTGGGGCCA AGGCACCCTC
1081 GTCACAGTGA GCTCCGGAGG CAGCGGAGGA AGCGGCGGCA GCGAGATCGT GCTGACCCAG
1141 TCCCCCGGCA CACTGTCCCT CAGCCCCGGC GAAAGAGCCA CACTGAGCTG TAGAGCTTCC
1201 CAGAGCGTGG GCAGCAGCTA TCTGGCTTGG TATCAGCAGA AGCCCGGCCA AGCCCCCAGA
1261 CTGCTCATCT ACGGAGCCTT CAGCAGAGCC ACCGGCATCC CCGACAGATT CAGCGGCAGC
1321 GGCAGCGGCA CCGATTTTAC CCTCACCATC TCCAGACTGG AGCCAGAGGA CTTCGCCGTG
1381 TATTACTGCC AACAGTACGG CAGCAGCCCT TGGACCTTTG GACAAGGCAC CAAGGTGGAA
1441 ATCAAACACC ACCACCATCA CCATCATCAT CACCAC
SEQ ID NO: 27
Ipilimumab VH and CH1 amino acid sequence
      1 QVQLVESGGG VVQPGRSLRL SCAASGFTES SYTMHWVRQA PGKGLEWVTF ISYDGNNKYY
    61 ADSVKGRFTI SRDNSKNTLY LOMNSLRAED TAIYYCARTG WLGPFDYWGQ GTLVTVSSAS
  121 TKGPSVFPLA PSSKSTSGGT AALGCLVKDY FPEPVTVSWN SGALTSGVHT FPAVLQSSGL
  181 YSLSSVVTVP SSSLGTQTYI CNVNHKPSNT KVDKKV
*SEQ ID NO: 27 optionally comprises the sequence (EPKSCDKT) (SEQ ID NO: 76) at
the C-terminal end, which corresponds to the hinge region for F(ab′)2 or Fab′,
wthe cysteine residue forms inter-chain bond with kappa light.
Alternatively, F(ab′)2 may comprise the sequence (e.g., proprietary
sequence as indicated in FIG. 10H) that forms inter-chain
dimerization domain C-terminal to SEQ ID NO: 27.
*See Table 3 and FIGS. 10D and 10H.
SEQ ID NO: 28
BioE2023 ipilimumab VL and CL amino acid sequence
      1 EIVLTQSPGT LSLSPGERAT LSCRASQSVG SSYLAWYQQK PGQAPRLLIY GAFSRATGIP
    61 DRFSGSGSGT DETLTISRLE PEDFAVYYCQ QYGSSPWTFG QGTKVEIKRT VAAPSVEIFP
  121 PSDEQLKSGT ASVVCLLNNE YPREAKVQWK VDNALQSGNS QESVTEQDSK DSTYSLSSTL
  181 TLSKADYEKH KVYACEVTHQ GLSSPVTKSF NRGEC
SEQ ID NO: 29
BioE2041 (anti-CTLA4 Tandem Biparatopic ScFv (TBS)) amino
acid sequence
VL IPI (G4S)3 VH IPI (KEA)6 VL 121 (G4S)3 VH 121 His tag (SEQ ID NOS 98,
102 and 98, respectively, in order of appearance)
EIVLTQSPGTLSLSPGERATLSCRASQSVGSSYLAWYQQKPGQAPRLLIYGAFSRATGIPDRESGSGSGTDETLT
ISRLEPEDFAVYYCQQYGSSPWTFGQGTKVEIKGGGGSGGGGSGGGGSQVQLVESGGGVVQPGRSLRLSCAASGF
TESSYTMHWVRQAPGKGLEWVTFISYDGNNKYYADSVKGRFTISRDNSKNTLYLQMNSLRAEDTAIYYCARTGWL
GPFDYWGQGTLVTVSSKEAKEAKEAKEAKEAKEADIQMTQSPSSLSASVGDRVTITCSATSSITYMSWYQQKPGK
APKLLIYDTSNLASGVPSRFSGSGSGTDYTLTISSLQPEDFATYYCQQWSSYPLTFGGGTKVEIKGGGGSGGGGS
GGGGSQVQLQESGPGLVKPSQTLSLTCTVSGFSLTSYGVYWVRQPPGKGLEWLGVIWAGGTTNYNSALMSRLTIS
KDTSKNQVSLKLSSVTAADTAVYYCARGPPHAMMKRGYAMDYWGQGTLVTVSS
*BioE2041 self-dimerizes.
*The underline represents the linker sequence.
SEQ ID NO: 30
BioE2042 (anti-CTLA4 Tandem Biparatopic ScFv (TBS)) amino
acid sequence
VL 121 (G4S)3 VH 121 (KEA)6 VL IPI (G4S)3 VH IPI His tag (SEQ ID NOS 98, 102 and
98, respectively, in order of appearance)
DIQMTQSPSSLSASVGDRVTITCSATSSITYMSWYQQKPGKAPKLLIYDTSNLASGVPSRESGSGSGTDYTLTIS
SLQPEDFATYYCQQWSSYPLTFGGGTKVEIKGGGGSGGGGSGGGGSQVQLQESGPGLVKPSQTLSLTCTVSGESL
TSYGVYWVRQPPGKGLEWLGVIWAGGTTNYNSALMSRLTISKDTSKNQVSLKLSSVTAADTAVYYCARGPPHAMM
KRGYAMDYWGQGTLVTVSSKEAKEAKEAKEAKEAKEAEIVLTQSPGTLSLSPGERATLSCRASQSVGSSYLAWYQ
QKPGQAPRLLIYGAFSRATGIPDRESGSGSGTDETLTISRLEPEDFAVYYCQQYGSSPWTFGQGTKVEIKGGGGS
GGGGSGGGGSQVQLVESGGGVVQPGRSLRLSCAASGFTFSSYTMHWVRQAPGKGLEWVTFISYDGNNKYYADSVK
GRFTISRDNSKNTLYLQMNSLRAEDTAIYYCARTGWLGPFDYWGQGTLVTVSS
*BioE2042 self-dimerizes.
*The underline represents the linker sequence.
BioE2061 (anti-CTLA4 Tandem Fab) amino acid sequence
VH 121--CH-(G4S)3--VH IPI-CH-His tag (VL 121--CL/VL IPI--CL) (“(G4S)3”
disclosed as SEQ ID NO: 98)
BioE2061 comprises 3 polypeptides with the sequence of SEQ ID NOs: 31-33.
See Wu et al. (2015) MAbs 7: 470-82
SEQ ID NO: 31
121-IPI Fab HC
QVQLQESGPGLVKPSQTLSLTCTVSGFSLTSYGVYWVRQPPGKGLEWLGVIWAGGTTNYNSALMSRLTISKDTSK
NQVSLKLSSVTAADTAVYYCARGPPHAMMKRGYAMDYWGQGTLVTVSSASTKGPSVFPLAPSSKSTSGGTAALGC
LVKDYFPEPVTVSWNSGALTSGVHTFPAVLQSSGLYSLSSVVTVPSSSLGTQTYICNVNHKPSNTKVDKKVEPKS
CDKTGGGGSGGGGSGGGGSQVQLVESGGGVVQPGRSLRLSCAASGFTFSSYTMHWVRQAPGKGLEWVTFISYDGN
NKYYADSVKGRFTISRDNSKNTLYLQMNSLRAEDTAIYYCARTGWLGPFDYWGQGTLVTVSSASTKGPSVFPLAP
SSKSTSGGTAALGCLVKDYFPEPVTVSWNSGALTSGVHTFPAVLQSSGLYSLSSVVTVPSSSLGTQTYICNVNHK
PSNTKVDKKVEPKSCDKTHHHHHHHHHH
SEQ ID NO: 32
121 LC
DIQMTQSPSSLSASVGDRVTITCSATSSITYMSWYQQKPGKAPKLLIYDTSNLASGVPSRESGSGSGTDYTLTIS
SLQPEDFATYYCQQWSSYPLTFGGGTKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGLSSPVTKSENRGEC
SEQ ID NO: 33
IPI LC
EIVLTQSPGTLSLSPGERATLSCRASQSVGSSYLAWYQQKPGQAPRLLIYGAFSRATGIPDRESGSGSGTDETLT
ISRLEPEDFAVYYCQQYGSSPWTFGQGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSENRGEC
BioE2062 (anti-CTLA4 Tandem Fab) amino acid sequence
VH IPI-CH-(G4S)3-VH 121--CH-His tag (VL IPI--CL/VL 121--CL) (“(G4S)3”
disclosed as SEQ ID NO: 98)
BioE2062 comprises 3 polypeptides with the sequence of SEQ ID NOs: 34-36.
See Wu et al.(2015) MAbs 7: 470-82
SEQ ID NO: 34
IPI-121 Fab HC
QVQLVESGGGVVQPGRSLRLSCAASGFTFSSYTMHWVRQAPGKGLEWVTFISYDGNNKYYADSVKGRFTISRDNS
KNTLYLQMNSLRAEDTAIYYCARTGWLGPFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKKVEPKSCDKTG
GGGSGGGGSGGGGSQVQLQESGPGLVKPSQTLSLTCTVSGFSLTSYGVYWVRQPPGKGLEWLGVIWAGGTTNYNS
ALMSRLTISKDTSKNQVSLKLSSVTAADTAVYYCARGPPHAMMKRGYAMDYWGQGTLVTVSSASTKGPSVFPLAP
SSKSTSGGTAALGCLVKDYFPEPVTVSWNSGALTSGVHTFPAVLQSSGLYSLSSVVTVPSSSLGTQTYICNVNHK
PSNTKVDKKVEPKSCDKT
SEQ ID NO: 35
IPI LC
EIVLTQSPGTLSLSPGERATLSCRASQSVGSSYLAWYQQKPGQAPRLLIYGAFSRATGIPDRESGSGSGTDETLT
ISRLEPEDFAVYYCQQYGSSPWTFGQGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSENRGEC
SEQ ID NO: 36
121 LC
DIQMTQSPSSLSASVGDRVTITCSATSSITYMSWYQQKPGKAPKLLIYDTSNLASGVPSRESGSGSGTDYTLTIS
SLQPEDFATYYCQQWSSYPLTFGGGTKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGLSSPVTKSENRGEC
BioE2091 (anti-CTLA4 Tandem Fab) amino acid sequence
VL IPI CL (G4S)3 VH 121 CH1 His tag (VH IPI CHI/VL 121 CL)
BioE2091 comprises 3 polypeptides with the sequence of SEQ ID NOs: 37-39.
See Wu et al. (2015) MAbs 7: 470-82
SEQ ID NO: 37
IPI LC-121 HC Fab
EIVLTQSPGTLSLSPGERATLSCRASQSVGSSYLAWYQQKPGQAPRLLIYGAFSRATGIPDRESGSGSGTDETLT
ISRLEPEDFAVYYCQQYGSSPWTFGQGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSENRGECGGGGSGGGGS
GGGGSQVQLQESGPGLVKPSQTLSLTCTVSGFSLTSYGVYWVRQPPGKGLEWLGVIWAGGTTNYNSALMSRLTIS
KDTSKNQVSLKLSSVTAADTAVYYCARGPPHAMMKRGYAMDYWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNTKVDKK
VEPKSCDKT
SEQ ID NO: 38
IPI HC Fab
QVQLVESGGGVVQPGRSLRLSCAASGFTFSSYTMHWVRQAPGKGLEWVTFISYDGNNKYYADSVKGRFTISRDNS
KNTLYLQMNSLRAEDTAIYYCARTGWLGPFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKKVEPKSCDKT
SEQ ID NO: 39
121 LC
DIQMTQSPSSLSASVGDRVTITCSATSSITYMSWYQQKPGKAPKLLIYDTSNLASGVPSRESGSGSGTDYTLTIS
SLQPEDFATYYCQQWSSYPLTFGGGTKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGLSSPVTKSENRGEC
BioE2092 (anti-CTLA4 Tandem Fab) amino acid sequence
VL 121 CL (G4S)3 VH IPI CHI His tag (VH 121 CH1/VL IPI CL)
BioE2092 comprises 3 polypeptides with the sequence of SEQ ID NOs: 40-42.
See Wu et al. (2015) MAbs 7: 470-82
SEQ ID NO: 40
121 LC-IPI HC Fab
DIQMTQSPSSLSASVGDRVTITCSATSSITYMSWYQQKPGKAPKLLIYDTSNLASGVPSRFSGSGSGTDYTLTIS
SLQPEDFATYYCQQWSSYPLTFGGGTKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGLSSPVTKSENRGECGGGGSGGGGSGG
GGSQVQLVESGGGVVQPGRSLRLSCAASGFTFSSYTMHWVRQAPGKGLEWVTFISYDGNNKYYADSVKGRFTISR
DNSKNTLYLQMNSLRAEDTAIYYCARTGWLGPFDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQTYICNVNHKPSNTKVDKKVEPKSCD
KT
SEQ ID NO: 41
121 HC Fab
QVQLQESGPGLVKPSQTLSLTCTVSGESLTSYGVYWVRQPPGKGLEWLGVIWAGGTTNYNSALMSRLTISKDTSK
NQVSLKLSSVTAADTAVYYCARGPPHAMMKRGYAMDYWGQGTLVTVSSASTKGPSVFPLAPSSKSTSGGTAALGC
LVKDYFPEPVTVSWNSGALTSGVHTFPAVLQSSGLYSLSSVVTVPSSSLGTQTYICNVNHKPSNTKVDKKVEPKS
CDKT
SEQ ID NO: 42
IPI LC
EIVLTQSPGTLSLSPGERATLSCRASQSVGSSYLAWYQQKPGQAPRLLIYGAFSRATGIPDRESGSGSGTDETLT
ISRLEPEDFAVYYCQQYGSSPWTFGQGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSENRGEC
BioE2111 (anti-CTLA4 DART) amino acid sequence
BioE2111 comprises 2 polypeptides with the sequence of SEQ ID NOs: 43-44.
SEQ ID NO: 43
VL IPI G3SG4 VH 121GGCGGGEVAALEKEVAALEKEVAALEKEVAALEK
(SEQ ID NOS 101 and 99)
EIVLTQSPGTLSLSPGERATLSCRASQSVGSSYLAWYQQKPGQAPRLLIYGAFSRATGIPDRESGSGSGTDETLT
ISRLEPEDFAVYYCQQYGSSPWTFGQGTKVEIKGGGSGGGGQVQLQESGPGLVKPSQTLSLTCTVSGESLTSYGV
YWVRQPPGKGLEWLGVIWAGGTTNYNSALMSRLTISKDTSKNQVSLKLSSVTAADTAVYYCARGPPHAMMKRGYA
MDYWGQGTLVTVSSGGCGGGEVAALEKEVAALEKEVAALEKEVAALEK
*The underline represents the linker sequence.
SEQ ID NO: 44
VL 121 G3SG4 VH IPI GGCGGGKVAALKEKVAALKEKVAALKEKVAALKE (SEQ ID
NOS 101 and 100)
DIQMTQSPSSLSASVGDRVTITCSATSSITYMSWYQQKPGKAPKLLIYDTSNLASGVPSRESGSGSGTDYTLTIS
SLQPEDFATYYCQQWSSYPLTFGGGTKVEIKGGGSGGGGQVQLVESGGGVVQPGRSLRLSCAASGFTESSYTMHW
VRQAPGKGLEWVTFISYDGNNKYYADSVKGRFTISRDNSKNTLYLQMNSLRAEDTAIYYCARTGWLGPFDYWGQG
TLVTVSSGGCGGGKVAALKEKVAALKEKVAALKEKVAALKE
*The underline represents the linker sequence.
BioE2012 (anti-CTLA4 Half antibody-scFv)
BioE2012 comprises 2 polypeptides with the sequence of SEQ ID NOs: 45-46.
IPI Fab Fc 121 ScFv [VL 121 (G4S)3 VH 121]
*BioE2012 contains the Fc-null LALAPG mutation
SEQ ID NO: 45
IPI 1/2Ab-121 scFv
QVQLVESGGGVVQPGRSLRLSCAASGFTFSSYTMHWVRQAPGKGLEWVTFISYDGNNKYYADSVKGRFTISRDNS
KNTLYLQMNSLRAEDTAIYYCARTGWLGPFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKKVEPKSCDKTH
TSPPSPAPEAAGGPSVFLFPPKPKDTLMISRTPEVTCVVVDVSHEDPEVKENWYVDGVEVHNAKTKPREEQYNST
YRVVSVLTVLHQDWLNGKEYKCKVSNKALGAPIEKTISKAKGQPREPQVYTLPPSRDELTKNQVSLTCLVKGFYP
SDIAVEWESNGQPENNYKTTPPVLDSDGSFRLYSRLTVDKSRWQQGNVFSCSVMHEALHNHYTQKSLSLSPGGGG
GSGGGGSDIQMTQSPSSLSASVGDRVTITCSATSSITYMSWYQQKPGKAPKLLIYDTSNLASGVPSRFSGSGSGT
DYTLTISSLQPEDFATYYCQQWSSYPLTFGGGTKVEIKGGGGSGGGGSGGGGSQVQLQESGPGLVKPSQTLSLTC
TVSGFSLTSYGVYWVRQPPGKGLEWLGVIWAGGTTNYNSALMSRLTISKDTSKNQVSLKLSSVTAADTAVYYCAR
GPPHAMMKRGYAMDYWGQGTLVTVSS
SEQ ID NO: 46
IPI LC
EIVLTQSPGTLSLSPGERATLSCRASQSVGSSYLAWYQQKPGQAPRLLIYGAFSRATGIPDRESGSGSGTDETLT
ISRLEPEDFAVYYCQQYGSSPWTFGQGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSENRGEC
BioE2081 (anti-CTLA4 Heterodimeric Fab/scFv-Fc)
BioE2081 comprises 3 polypeptides with the sequence of SEQ ID NOs: 47-49.
121 ScFv Fc IPI Fab Fc (Fc silent) KiH Fc
*KiH stands for Knob in Hole. See e.g., Ridgway et al. (1996) Protein
Engineering 9: 617-621
*BioE2081 contains the Fc-silent LALAPG mutation
SEQ ID NO: 47
IPI Fab-knob Fc
QVQLVESGGGVVQPGRSLRLSCAASGFTESSYTMHWVRQAPGKGLEWVTFISYDGNNKYYADSVKGRFTISRDNS
KNTLYLQMNSLRAEDTAIYYCARTGWLGPFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKKVEPKSCDKTH
TCPPCPAPEAAGGPSVFLFPPKPKDTLMISRTPEVTCVVVDVSHEDPEVKENWYVDGVEVHNAKTKPREEQYNST
YRVVSVLTVLHQDWLNGKEYKCKVSNKALGAPIEKTISKAKGQPREPQVYTLPPCRDELTKNQVSLWCLVKGFYP
SDIAVEWESNGQPENNYKTTPPVLDSDGSFFLYSKLTVDKSRWQQGNVFSCSVMHEALHNHYTQKSLSLSPG
SEQ ID NO: 48
121 scFv-hole Fc
DIQMTQSPSSLSASVGDRVTITCSATSSITYMSWYQQKPGKAPKLLIYDTSNLASGVPSRESGSGSGTDYTLTIS
SLQPEDFATYYCQQWSSYPLTFGGGTKVEIKGGGGSGGGGSGGGGSQVQLQESGPGLVKPSQTLSLTCTVSGESL
TSYGVYWVRQPPGKGLEWLGVIWAGGTTNYNSALMSRLTISKDTSKNQVSLKLSSVTAADTAVYYCARGPPHAMM
KRGYAMDYWGQGTLVTVSSGGGGSEPKSQDKTHTCPPCPAPEAAGGPSVFLFPPKPKDTLMISRTPEVTCVVVDV
SHEDPEVKENWYVDGVEVHNAKTKPREEQYNSTYRVVSVLTVLHQDWLNGKEYKCKVSNKALGAPIEKTISKAKG
QPREPQVCTLPPSRDELTKNQVSLSCAVKGFYPSDIAVEWESNGQPENNYKTTPPVLDSDGSFFLVSKLTVDKSR
WQQGNVFSCSVMHEALHNHYTQKSLSLSPGK
SEQ ID NO: 49
IPI LC
EIVLTQSPGTLSLSPGERATLSCRASQSVGSSYLAWYQQKPGQAPRLLIYGAFSRATGIPDRFSGSGSGTDETLT
ISRLEPEDFAVYYCQQYGSSPWTFGQGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSENRGEC
BioE2082 (anti-CTLA4 Heterodimeric Fab/scFv-Fc)
BioE2082 comprises 3 polypeptides with the sequence of SEQ ID NOs: 50-52.
121 Fab Fc IPI ScFv Fc (Fc silent) KiH Fc
*KiH stands for Knob in Hole. See e.g., Ridgway et al. (1996) Protein
Engineering 9: 617-621
*BioE2082 contains the Fc-silent LALAPG mutation
SEQ ID NO: 50
121 Fab-knob Fc
QVQLQESGPGLVKPSQTLSLTCTVSGFSLTSYGVYWVRQPPGKGLEWLGVIWAGGTTNYNSALMSRLTISKDTSK
NQVSLKLSSVTAADTAVYYCARGPPHAMMKRGYAMDYWGQGTLVTVSSASTKGPSVFPLAPSSKSTSGGTAALGC
LVKDYFPEPVTVSWNSGALTSGVHTFPAVLQSSGLYSLSSVVTVPSSSLGTQTYICNVNHKPSNTKVDKKVEPKS
CDKTHTCPPCPAPEAAGGPSVFLFPPKPKDTLMISRTPEVTCVVVDVSHEDPEVKENWYVDGVEVHNAKTKPREE
QYNSTYRVVSVLTVLHQDWLNGKEYKCKVSNKALGAPIEKTISKAKGQPREPQVYTLPPCRDELTKNQVSLWCLV
KGFYPSDIAVEWESNGQPENNYKTTPPVLDSDGSFFLYSKLTVDKSRWQQGNVFSCSVMHEALHNHYTQKSLSLS
PG
SEQ ID NO: 51
IPI scFv-hole Fc
EIVLTQSPGTLSLSPGERATLSCRASQSVGSSYLAWYQQKPGQAPRLLIYGAFSRATGIPDRESGSGSGTDETLT
ISRLEPEDFAVYYCQQYGSSPWTFGQGTKVEIKGGGGSGGGGGGGGSQVQLVESGGGVVQPGRSLRLSCAASGF
TFSSYTMHWVRQAPGKGLEWVTFISYDGNNKYYADSVKGRFTISRDNSKNTLYLQMNSLRAEDTAIYYCARTGWL
GPFDYWGQGTLVTVSSGGGGSEPKSQDKTHTCPPCPAPEAAGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALGAPIEKTISKAKGQPR
EPQVCTLPPSRDELTKNQVSLSCAVKGFYPSDIAVEWESNGQPENNYKTTPPVLDSDGSFFLVSKLTVDKSRWQQ
GNVFSCSVMHEALHNHYTQKSLSLSPGK
SEQ ID NO: 52
121 LC
DIQMTQSPSSLSASVGDRVTITCSATSS ITYMSWYQQKPGKAPKLLIYDTSNLASGVPSRESGSGSGTDYTLTIS
SLOPEDFATYYCQQWSSYPLTFGGGTKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGLSSPVTKSENRGEC
BioE2121 (anti-CTLA4 Heterodimeric scFvs)
BioE2121 comprises 2 polypeptides with the sequence of SEQ ID NOs: 53-54.
IPI scFv-Fc/121 scFv-Fc (KiH)
*KiH stands for Knob in Hole. See e.g., Ridgway et al. (1996) Protein
Engineering 9: 617-621
*BioE2121 contains the Fc-silent LALAPG mutation
SEQ ID NO: 53
IPI scFv-knob Fc
EIVLTQSPGTLSLSPGERATLSCRASQSVGSSYLAWYQQKPGQAPRLLIYGAFSRATGIPDRESGSGSGTDFTLT
ISRLEPEDFAVYYCQQYGSSPWTFGQGTKVEIKGGGGSGGGGGGGGSQVQLVESGGGVVQPGRSLRLSCAASGE
TESSYTMHWVRQAPGKGLEWVTFISYDGNNKYYADSVKGRFTISRDNSKNTLYLQMNSLRAEDTAIYYCARTGWL
GPFDYWGQGTLVTVSSGGGGSEPKSCDKTHTCPPCPAPEAAGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALGAPIEKTISKAKGQPR
EPQVYTLPPCRDELTKNQVSLWCLVKGFYPSDIAVEWESNGQPENNYKTTPPVLDSDGSFFLYSKLTVDKSRWQQ
GNVFSCSVMHEALHNHYTQKSLSLSPG
SEQ ID NO: 54
121 scFv-hole Fc
DIQMTQSPSSLSASVGDRVTITCSATSSITYMSWYQQKPGKAPKLLIYDTSNLASGVPSRESGSGSGTDYTLTIS
SLQPEDFATYYCQQWSSYPLTFGGGTKVEIKGGGGSGGGGSGGGGSQVQLQESGPGLVKPSQTLSLTCTVSGESL
TSYGVYWVRQPPGKGLEWLGVIWAGGTTNYNSALMSRLTISKDTSKNQVSLKLSSVTAADTAVYYCARGPPHAMM
KRGYAMDYWGQGTLVTVSSGGGGSEPKSQDKTHTCPPCPAPEAAGGPSVFLFPPKPKDTLMISRTPEVTCVVVDV
SHEDPEVKENWYVDGVEVHNAKTKPREEQYNSTYRVVSVLTVLHQDWLNGKEYKCKVSNKALGAPIEKTISKAKG
QPREPQVCTLPPSRDELTKNQVSLSCAVKGFYPSDIAVEWESNGQPENNYKTTPPVLDSDGSFFLVSKLTVDKSR
WQQGNVESCSVMHEALHNHYTQKSLSLSPGK
SEQ ID NO: 59
BioE2410 amino acid sequence
QVQLVESGGGVVQPGRSLRLSCAASGFTFSSYTMHWVRQAPGKGLEWVTFISYDGNNKYYADSVKGRFTISRDNS
KNTLYLQMNSLRAEDTAIYYCARTGWLGPFDYWGQGTLVTVSSGGSGGSGGSDIQMTQSPSSLSASVGDRVTITC
SATSSITYMSWYQQKPGKAPKLLIYDTSNLASGVPSRESGSGSGTDYTLTISSLQPEDFATYYCQQWSSYPLTFG
GGTKVEIKGGSGGSGGSQVQLQESGPGLVKPSQTLSLTCTVSGFSLTSYGVYWVRQPPGKGLEWLGVIWAGGTTN
YNSALMSRLTISKDTSKNQVSLKLSSVTAADTAVYYCARGPPHAMMKRGYAMDYWGQGTLVTVSSGGSGGSGGSE
IVLTQSPGTLSLSPGERATLSCRASQSVGSSYLAWYQQKPGQAPRLLIYGAFSRATGIPDRESGSGSGTDETLTI
SRLEPEDFAVYYCQQYGSSPWTFGQGTKVEIKGGSGGDKTHTCPPCPAPELLGGPSVFLFPPKPKDTLMISRTPE
VTCVVVDVSHEDPEVKENWYVDGVEVHNAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEWESNGQPENNYKTTPPVLDSDGSFFLYS
KLTVDKSRWQQGNVFSCSVMHEALHNHYTQKSLSLSPGK
SEQ ID NO: 60
BioE2410 nucleic acid sequence
CAAGTGCAGCTGGTGGAGAGCGGTGGCGGCGTGGTGCAGCCCGGTAGAAGCCTGAGACTGAGCTGCGCCGCAAGC
GGCTTCACCTTCAGCAGCTACACCATGCACTGGGTGAGACAAGCACCCGGCAAGGGACTGGAATGGGTCACGTTC
ATCAGCTACGACGGCAACAACAAGTACTACGCCGACAGCGTGAAGGGCAGATTCACCATCAGCCGAGACAACAGC
AAGAACACCCTGTACCTGCAGATGAACAGCCTGAGAGCCGAGGACACCGCCATCTACTATTGCGCTAGAACGGGA
TGGCTGGGCCCCTTCGATTATTGGGGGCAAGGCACGTTGGTGACCGTCTCAAGCGGCGGAAGCGGAGGCAGCGGA
GGATCGGATATACAGATGACACAGTCGCCTAGCTCACTGAGCGCAAGCGTGGGCGACAGAGTGACCATCACCTGC
AGCGCCACAAGCAGCATCACCTACATGAGCTGGTATCAGCAAAAACCCGGCAAGGCCCCCAAGCTGCTGATCTAC
GATACAAGCAACCTGGCAAGCGGCGTGCCTAGCAGATTCTCTGGCAGCGGCAGCGGCACCGACTATACTCTCACC
ATAAGCAGTCTACAGCCTGAGGATTTCGCCACCTACTATTGTCAGCAGTGGTCGAGCTACCCCCTGACCTTCGGG
GGCGGCACCAAGGTAGAGATTAAAGGTGGGAGCGGCGGGAGTGGTGGCAGCCAAGTCCAACTGCAAGAGTCCGGA
CCCGGTCTTGTGAAGCCTTCTCAGACTCTTAGCTTAACATGCACCGTGAGCGGCTTCAGCCTTACAAGCTACGGC
GTCTACTGGGTACGTCAACCACCTGGTAAGGGTTTAGAATGGCTAGGGGTGATCTGGGCCGGCGGCACCACCAAC
TACAACAGCGCCCTGATGAGCAGACTGACGATCTCTAAGGACACTAGCAAAAACCAAGTGAGCCTCAAGCTGAGC
AGCGTGACCGCCGCCGACACCGCCGTATACTACTGTGCAAGAGGCCCCCCCCACGCCATGATGAAGAGAGGCTAC
GCCATGGATTACTGGGGGCAAGGCACTCTAGTGACCGTGAGTAGTGGCGGGAGCGGTGGGAGCGGCGGTTCCGAA
ATCGTGCTGACACAGAGCCCTGGCACTCTGTCTCTGAGTCCCGGCGAGAGAGCTACCCTGAGCTGCAGAGCATCT
CAGAGCGTGGGCAGCAGCTACCTGGCCTGGTATCAGCAAAAGCCCGGCCAAGCCCCTAGACTTCTGATCTACGGC
GCATTCAGCAGAGCCACCGGCATCCCCGACAGATTCAGTGGATCTGGCAGCGGAACGGACTTCACCCTAACAATC
AGCCGTCTGGAACCCGAAGACTTTGCGGTGTATTACTGTCAACAGTACGGTAGCAGCCCCTGGACCTTCGGCCAA
GGCACAAAGGTGGAGATCAAAGGCGGTTCCGGTGGCGACAAAACACACACCTGTCCCCCCTGCCCCGCCCCTGAG
CTGTTAGGTGGTCCTAGCGTGTTCCTGTTCCCCCCCAAGCCCAAGGACACCCTGATGATCAGCAGAACCCCTGAG
GTGACCTGCGTGGTGGTGGACGTGAGCCACGAGGACCCTGAGGTGAAGTTCAACTGGTACGTGGACGGCGTGGAG
GTGCACAACGCCAAGACCAAGCCTAGAGAGGAGCAGTACAACAGCACCTACAGAGTGGTGAGCGTGCTGACCGTG
CTGCACCAAGACTGGCTGAACGGCAAGGAGTACAAGTGCAAGGTGAGCAACAAGGCCCTGCCCGCCCCCATCGAG
AAGACCATCAGCAAGGCCAAGGGGCAGCCTAGAGAGCCCCAAGTGTACACCCTGCCCCCTAGCAGAGACGAGCTG
ACCAAGAACCAAGTGTCGCTAACCTGTTTGGTGAAGGGCTTCTACCCTAGCGACATCGCCGTGGAGTGGGAGAGC
AACGGGCAGCCTGAGAACAACTACAAGACCACCCCCCCCGTGCTGGACAGCGACGGCAGCTTCTTCCTGTACAGC
AAGCTGACCGTGGACAAGAGCAGATGGCAGCAAGGCAACGTGTTCAGCTGCAGCGTGATGCACGAGGCCCTGCAC
AACCACTACACACAGAAGAGCCTGTCGCTGAGCCCCGGAAAA
SEQ ID NO: 61
BioE2420 amino acid sequence
QVQLVESGGGVVQPGRSLRLSCAASGFTFSSYTMHWVRQAPGKGLEWVTFISYDGNNKYYADSVKGRFTISRDNS
KNTLYLQMNSLRAEDTAIYYCARTGWLGPFDYWGQGTLVTVSSGGSGGSGGSDIQMTQSPSSLSASVGDRVTITC
SATSSITYMSWYQQKPGKAPKLLIYDTSNLASGVPSRESGSGSGTDYTLTISSLQPEDFATYYCQQWSSYPLTFG
GGTKVEIKGGSGGSGGSQVQLQESGPGLVKPSQTLSLTCTVSGFSLTSYGVYWVRQPPGKGLEWLGVIWAGGTTN
YNSALMSRLTISKDTSKNQVSLKLSSVTAADTAVYYCARGPPHAMMKRGYAMDYWGQGTLVTVSSGGSGGSGGSE
IVLTQSPGTLSLSPGERATLSCRASQSVGSSYLAWYQQKPGQAPRLLIYGAFSRATGIPDRESGSGSGTDFTLTI
SRLEPEDFAVYYCQQYGSSPWTFGQGTKVEIKGGSGGDKTHTCPPCPAPEAAGGPSVFLFPPKPKDTLMISRTPE
VTCVVVDVSHEDPEVKFNWYVDGVEVHNAKTKPREEQYNSTYRVVSVLTVLHQDWLNGKEYKCKVSNKALGAPIE
KTISKAKGQPREPQVYTLPPSRDELTKNQVSLTCLVKGFYPSDIAVEWESNGQPENNYKTTPPVLDSDGSFFLYS
KLTVDKSRWQQGNVFSCSVMHEALHNHYTQKSLSLSPGK
SEQ ID NO: 62
BioE2420 nucleic acid sequence
CAAGTGCAGCTGGTGGAGAGCGGTGGCGGCGTGGTGCAGCCCGGTAGAAGCCTGAGACTGAGCTGCGCCGCAAGC
GGCTTCACCTTCAGCAGCTACACCATGCACTGGGTGAGACAAGCACCCGGCAAGGGACTGGAATGGGTCACGTTC
ATCAGCTACGACGGCAACAACAAGTACTACGCCGACAGCGTGAAGGGCAGATTCACCATCAGCCGAGACAACAGC
AAGAACACCCTGTACCTGCAGATGAACAGCCTGAGAGCCGAGGACACCGCCATCTACTATTGCGCTAGAACGGGA
TGGCTGGGCCCCTTCGATTATTGGGGGCAAGGCACGTTGGTGACCGTCTCAAGCGGCGGAAGCGGAGGCAGCGGA
GGATCGGATATACAGATGACACAGTCGCCTAGCTCACTGAGCGCAAGCGTGGGCGACAGAGTGACCATCACCTGC
AGCGCCACAAGCAGCATCACCTACATGAGCTGGTATCAGCAAAAACCCGGCAAGGCCCCCAAGCTGCTGATCTAC
GATACAAGCAACCTGGCAAGCGGCGTGCCTAGCAGATTCTCTGGCAGCGGCAGCGGCACCGACTATACTCTCACC
ATAAGCAGTCTACAGCCTGAGGATTTCGCCACCTACTATTGTCAGCAGTGGTCGAGCTACCCCCTGACCTTCGGG
GGCGGCACCAAGGTAGAGATTAAAGGTGGGAGCGGCGGGAGTGGTGGCAGCCAAGTCCAACTGCAAGAGTCCGGA
CCCGGTCTTGTGAAGCCTTCTCAGACTCTTAGCTTAACATGCACCGTGAGCGGCTTCAGCCTTACAAGCTACGGC
GTCTACTGGGTACGTCAACCACCTGGTAAGGGTTTAGAATGGCTAGGGGTGATCTGGGCCGGCGGCACCACCAAC
TACAACAGCGCCCTGATGAGCAGACTGACGATCTCTAAGGACACTAGCAAAAACCAAGTGAGCCTCAAGCTGAGC
AGCGTGACCGCCGCCGACACCGCCGTATACTACTGTGCAAGAGGCCCCCCCCACGCCATGATGAAGAGAGGCTAC
GCCATGGATTACTGGGGGCAAGGCACTCTAGTGACCGTGAGTAGTGGCGGGAGCGGTGGGAGCGGCGGTTCCGAA
ATCGTGCTGACACAGAGCCCTGGCACTCTGTCTCTGAGTCCCGGCGAGAGAGCTACCCTGAGCTGCAGAGCATCT
CAGAGCGTGGGCAGCAGCTACCTGGCCTGGTATCAGCAAAAGCCCGGCCAAGCCCCTAGACTTCTGATCTACGGC
GCATTCAGCAGAGCCACCGGCATCCCCGACAGATTCAGTGGATCTGGCAGCGGAACGGACTTCACCCTAACAATC
AGCCGTCTGGAACCCGAAGACTTTGCGGTGTATTACTGTCAACAGTACGGTAGCAGCCCCTGGACCTTCGGCCAA
GGCACAAAGGTGGAGATCAAAGGCGGTTCCGGTGGCGACAAAACACACACCTGTCCCCCCTGCCCCGCCCCTGAG
GCTGCCGGTGGTCCTAGCGTGTTCCTGTTCCCCCCCAAGCCCAAGGACACCCTGATGATCAGCAGAACCCCTGAG
GTGACCTGCGTGGTGGTGGACGTGAGCCACGAGGACCCTGAGGTGAAGTTCAACTGGTACGTGGACGGCGTGGAG
GTGCACAACGCCAAGACCAAGCCTAGAGAGGAGCAGTACAACAGCACCTACAGAGTGGTGAGCGTGCTGACCGTG
CTGCACCAAGACTGGCTGAACGGCAAGGAGTACAAGTGCAAGGTGAGCAACAAGGCCCTGGGCGCCCCCATCGAG
AAGACCATCAGCAAGGCCAAGGGGCAGCCTAGAGAGCCCCAAGTGTACACCCTGCCCCCTAGCAGAGACGAGCTG
ACCAAGAACCAAGTGTCGCTAACCTGTTTGGTGAAGGGCTTCTACCCTAGCGACATCGCCGTGGAGTGGGAGAGC
AACGGGCAGCCTGAGAACAACTACAAGACCACCCCCCCCGTGCTGGACAGCGACGGCAGCTTCTTCCTGTACAGC
AAGCTGACCGTGGACAAGAGCAGATGGCAGCAAGGCAACGTGTTCAGCTGCAGCGTGATGCACGAGGCCCTGCAC
AACCACTACACACAGAAGAGCCTGTCGCTGAGCCCCGGAAAA
SEQ ID NO: 63
BioE2430 amino acid sequence
QVQLVESGGGVVQPGRSLRLSCAASGFTFSSYTMHWVRQAPGKGLEWVTFISYDGNNKYYADSVKGRFTISRDNS
KNTLYLQMNSLRAEDTAIYYCARTGWLGPFDYWGQGTLVTVSSGGSGGSGGSDIQMTQSPSSLSASVGDRVTITC
SATSSITYMSWYQQKPGKAPKLLIYDTSNLASGVPSRFSGSGSGTDYTLTISSLQPEDFATYYCQQWSSYPLTFG
GGTKVEIKGGSGGSGGSQVQLQESGPGLVKPSQTLSLTCTVSGFSLTSYGVYWVRQPPGKGLEWLGVIWAGGTTN
YNSALMSRLTISKDTSKNQVSLKLSSVTAADTAVYYCARGPPHAMMKRGYAMDYWGQGTLVTVSSGGSGGSGGSE
IVLTQSPGTLSLSPGERATLSCRASQSVGSSYLAWYQQKPGQAPRLLIYGAFSRATGIPDRESGSGSGTDETLTI
SRLEPEDFAVYYCQQYGSSPWTFGQGTKVEIKGGSGGEAHKSEIAHRYNDLGEQHFKGLVLIAFSQYLQKCSYDE
HAKLVQEVTDFAKTCVADESAANCDKSLHTLFGDKLCAIPNLRENYGELADCCTKQEPERNECFLQHKDDNPSLP
PFERPEAEAMCTSFKENPTTEMGHYLHEVARRHPYFYAPELLYYAEQYNEILTQCCAEADKESCLTPKLDGVKEK
ALVSSVRQRMKCSSMQKFGERAFKAWAVARLSQTFPNADFAEITKLATDLTKVNKECCHGDLLECADDRAELAKY
MCENQATISSKLQTCCDKPLLKKAHCLSEVEHDTMPADLPAIAADFVEDQEVCKNYAEAKDVELGTFLYEYSRRH
PDYSVSLLLRLAKKYEATLEKCCAEANPPACYGTVLAEFQPLVEEPKNLVKTNCDLYEKLGEYGFQNAILVRYTQ
KAPQVSTPTLVEAARNLGRVGTKCCTLPEDQRLPCVEDYLSAILNRVCLLHEKTPVSEHVTKCCSGSLVERRPCE
SALTVDETYVPKEFKAETFTFHSDICTLPEKEKQIKKQTALAELVKHKPKATAEQLKTVMDDFAQFLDTCCKAAD
KDTCFSTEGPNLVTRCKDALAHHHHHHHHHH
SEQ ID NO: 64
BioE2430 nucleic acid sequence
CAAGTGCAATTGGTAGAGTCCGGCGGCGGCGTTGTGCAACCCGGCAGAAGCCTTAGACTGAGCTGCGCCGCAAGC
GGCTTCACCTTCAGCAGCTACACCATGCACTGGGTGAGACAAGCCCCCGGCAAGGGCCTGGAATGGGTGACCTTC
ATCAGCTACGACGGCAACAACAAGTACTACGCTGATAGCGTGAAGGGCAGATTCACTATCAGCCGTGACAACAGC
AAGAACACCCTGTACCTGCAGATGAACAGCCTGAGAGCCGAGGACACCGCCATCTACTACTGCGCAAGAACCGGC
TGGCTGGGCCCCTTCGACTACTGGGGCCAAGGGACATTAGTTACTGTTAGCAGCGGCGGGAGCGGGGGCAGCGGA
GGATCGGATATACAGATGACACAGAGCCCTAGCAGCCTGAGCGCAAGCGTGGGCGACAGAGTGACCATCACCTGC
AGCGCCACAAGCAGCATCACCTACATGAGCTGGTATCAGCAAAAACCCGGCAAGGCCCCCAAGCTGCTGATCTAC
GACACAAGCAACCTGGCAAGCGGCGTGCCTAGCAGATTCTCTGGCAGCGGCAGCGGCACAGATTATACTCTCACA
ATCAGCAGTCTGCAGCCTGAGGATTTCGCCACATACTACTGCCAACAGTGGAGCAGCTACCCCCTGACCTTCGGC
GGCGGCACTAAAGTAGAGATTAAAGGCGGCAGCGGTGGAAGTGGCGGGAGCCAAGTGCAACTGCAAGAGAGCGGT
CCCGGCCTGGTGAAGCCTTCTCAGACCTTGAGCCTTACCTGCACCGTGAGCGGCTTCAGCCTGACAAGCTACGGC
GTGTACTGGGTGCGGCAACCCCCCGGCAAAGGCTTGGAATGGCTGGGGGTGATCTGGGCCGGCGGCACCACCAAC
TACAACAGCGCCCTGATGAGCAGACTGACCATTAGCAAGGACACAAGCAAGAACCAAGTGAGCCTGAAGCTATCT
AGCGTGACCGCCGCCGACACCGCCGTCTATTACTGTGCAAGAGGCCCCCCCCACGCCATGATGAAGAGAGGCTAC
GCCATGGATTATTGGGGCCAAGGCACCCTGGTTACCGTCTCTTCCGGGGGTAGCGGCGGCAGCGGAGGTTCCGAG
ATCGTGTTAACTCAGAGCCCTGGCACCCTGTCGCTGAGCCCTGGTGAAAGGGCTACGCTAAGCTGCAGAGCATCT
CAGAGCGTGGGCAGCAGCTACCTGGCCTGGTATCAGCAGAAACCCGGCCAAGCCCCTAGACTGCTGATCTATGGT
GCCTTCAGCAGAGCCACCGGCATCCCCGACAGATTCAGCGGTTCCGGTAGTGGCACAGACTTCACTCTGACCATC
AGCCGGCTAGAACCTGAGGATTTTGCCGTTTACTACTGTCAGCAGTATGGCAGCAGCCCCTGGACCTTCGGCCAA
GGCACGAAGGTGGAGATAAAAGGTGGAAGCGGCGGGGAAGCCCACAAGAGCGAGATCGCCCACAGATACAACGAC
CTGGGCGAGCAGCACTTCAAGGGCCTGGTGCTGATCGCCTTCTCTCAGTACCTGCAGAAGTGCAGCTACGACGAG
CACGCCAAGCTGGTGCAAGAGGTGACCGACTTCGCCAAGACCTGCGTGGCCGACGAGAGCGCCGCCAACTGCGAC
AAGAGCCTGCACACCCTGTTCGGCGACAAGCTGTGCGCCATCCCCAACCTGAGAGAGAACTACGGCGAGCTGGCC
GACTGCTGCACCAAGCAAGAGCCGGAGAGAAATGAGTGCTTCCTGCAGCACAAGGACGACAACCCTAGCCTGCCC
CCCTTCGAGAGACCTGAGGCCGAGGCCATGTGCACTAGCTTCAAGGAGAACCCCACCACCTTCATGGGCCACTAC
CTGCACGAGGTGGCTAGACGTCATCCATATTTCTACGCCCCTGAGCTGCTGTACTATGCAGAGCAGTACAACGAG
ATCCTGACCCAATGTTGTGCCGAGGCGGACAAAGAGAGCTGCCTGACCCCCAAGCTGGACGGCGTGAAGGAGAAG
GCCCTGGTGAGCAGCGTCAGACAGAGAATGAAGTGCAGCAGCATGCAGAAGTTCGGGGAGAGAGCTTTCAAGGCC
TGGGCCGTGGCTAGACTGTCTCAGACCTTCCCCAACGCCGACTTCGCCGAGATCACCAAGCTGGCCACCGACCTG
ACCAAGGTGAACAAGGAGTGCTGCCACGGCGACCTGCTGGAGTGCGCCGACGACAGAGCCGAGCTGGCCAAGTAC
ATGTGCGAGAACCAAGCCACTATCTCATCCAAATTACAGACCTGCTGCGACAAGCCCCTGCTGAAGAAAGCTCAT
TGCCTCAGCGAGGTGGAGCATGACACCATGCCCGCCGATCTGCCCGCCATCGCCGCCGACTTCGTGGAGGACCAA
GAGGTGTGCAAGAACTACGCCGAGGCGAAGGATGTGTTCCTGGGCACCTTCCTGTACGAGTACAGCCGAAGGCAC
CCCGACTACAGCGTGAGCCTGCTGCTGAGACTGGCCAAGAAGTACGAGGCCACTTTGGAGAAATGCTGCGCTGAG
GCAAACCCCCCTGCCTGCTACGGCACCGTGCTGGCCGAGTTTCAGCCCCTGGTAGAGGAGCCGAAGAACCTGGTG
AAGACCAACTGCGACCTGTACGAGAAGCTGGGCGAGTACGGCTTTCAGAACGCCATCCTGGTGAGATACACACAG
AAGGCCCCCCAAGTGAGCACCCCCACCTTGGTGGAAGCCGCAAGAAACCTGGGCAGAGTGGGCACCAAATGTTGC
ACGCTGCCGGAGGACCAAAGACTGCCCTGCGTGGAGGACTACCTGAGCGCCATCCTGAACAGAGTGTGCCTGCTG
CACGAGAAGACCCCCGTGAGCGAGCACGTGACCAAGTGCTGCAGTGGCAGCCTGGTGGAGAGAAGACCCTGCTTC
AGCGCCCTGACCGTGGACGAGACCTACGTGCCCAAGGAGTTCAAGGCCGAGACCTTCACCTTCCACAGCGACATC
TGTACTCTCCCTGAGAAGGAGAAGCAGATCAAGAAGCAGACCGCCCTGGCCGAGCTGGTGAAGCACAAGCCCAAG
GCCACCGCCGAGCAGCTGAAGACCGTGATGGACGACTTCGCTCAGTTCCTGGACACCTGCTGCAAGGCCGCCGAC
AAGGACACCTGCTTCAGCACCGAGGGCCCCAACCTGGTGACAAGATGCAAGGACGCCCTGGCCCACCACCACCAC
CACCACCACCACCACCAC
SEQ ID NO: 65
BioE2440 VH amino acid sequence
DVQLQESGPGLVKPSQSLSLTCSVTGYSITSGYVWNWIRQFPGNKLEWMGYISHDGNTNYNPSLKNRISITRDTS
KNQFFLKLNSVTTEDSATYYCTRNYGYGGTMDYWGQGTAVSVSSAKTTAPSVYPLAPVCGDTTGSSVTLGCLVKG
YFPEPVTLTWNSGSLSSGVHTFPAVLQSDLYTLSSSVTVTSSTWPSQSITCNVAHPASSTKVDKKIEPRGPTIKP
CPPCKCPAPNAAGGPSVFIFPPKIKDVLMISLSPIVTCVVVDVSEDDPDVQISWFVNNVEVHTAQTQTHREDYNS
TLRVVSALPIQHQDWMSGKEFKCKVNNKDLGAPIERTISKPKGSVRAPQVYVLPPPEEEMTKKQVTLTCMVTDEM
PEDIYVEWTNNGKTELNYKNTEPVLDSDGSYFMYSKLRVEKKNWVERNSYSCSVVHEGLHNHHTTKSFSRTPGK
SEQ ID NO: 66
BioE2440 VH nucleic acid sequence
GACGTGCAGCTGCAAGAGAGCGGCCCCGGCCTGGTGAAGCCGAGCCAATCTCTGAGCCTGACCTGCAGTGTGACG
GGATACTCAATCACCTCTGGTTACGTGTGGAACTGGATCAGACAGTTCCCCGGCAACAAGCTGGAGTGGATGGGC
TACATCAGCCACGACGGCAACACCAACTACAACCCTAGCCTGAAGAACAGAATCAGCATAACAAGAGACACAAGC
AAGAATCAGTTCTTCCTGAAGCTGAACAGCGTGACCACCGAGGACAGCGCCACCTACTACTGCACAAGAAACTAC
GGCTACGGCGGCACCATGGACTACTGGGGCCAAGGCACCGCCGTGAGCGTAAGCAGCGCCAAGACAACAGCCCCT
AGCGTGTACCCGCTGGCCCCCGTTTGCGGCGACACCACCGGTAGCAGCGTGACCCTGGGGTGCCTGGTGAAGGGC
TACTTCCCTGAGCCCGTGACCCTGACTTGGAATAGCGGCAGCCTGAGCAGCGGCGTGCACACCTTCCCCGCCGTG
CTGCAGAGCGACCTGTACACCCTGAGCAGCAGCGTGACAGTGACGAGCAGCACCTGGCCTAGCCAAAGTATCACC
TGCAACGTGGCCCACCCCGCAAGCAGCACCAAGGTGGACAAGAAGATCGAACCAAGAGGCCCCACTATTAAACCT
TGCCCCCCCTGCAAGTGTCCCGCACCCAATGCAGCAGGCGGCCCTAGCGTGTTCATCTTCCCCCCCAAGATCAAG
GACGTGCTGATGATCAGCCTGAGCCCCATCGTGACCTGCGTGGTGGTGGACGTGAGCGAGGACGACCCCGACGTG
CAGATCAGCTGGTTCGTGAACAACGTGGAGGTGCACACCGCTCAGACACAGACCCACAGAGAGGACTACAACAGC
ACCCTGAGAGTGGTGAGCGCCCTGCCCATTCAGCACCAAGACTGGATGAGCGGCAAGGAGTTCAAGTGCAAGGTG
AACAACAAGGACCTGGGCGCCCCCATCGAGAGAACCATCAGCAAGCCCAAGGGCAGCGTGAGAGCCCCCCAAGTG
TACGTGCTGCCCCCCCCTGAGGAGGAGATGACCAAGAAGCAAGTGACCCTAACCTGTATGGTGACCGACTTCATG
CCTGAGGACATCTACGTGGAGTGGACCAACAACGGCAAGACCGAGCTGAACTACAAGAACACCGAGCCCGTGCTG
GACAGCGACGGCAGCTACTTCATGTACAGCAAGCTGAGAGTGGAGAAGAAGAACTGGGTGGAGAGAAACAGCTAC
AGCTGCAGCGTGGTGCACGAGGGCCTGCACAACCACCACACCACCAAGAGCTTCAGCAGAACCCCCGGCAAG
SEQ ID NO: 67
BioE2440 VL amino acid sequence
DIVMTQSQKFMSTSVGDRVSVTCKASQNVGTYVAWYEQKLGQSPKALIFSASYRYTGVPDRFTGSGSGTDETLTI
SNVQSEDLAEYFCQQYDSYPLTFGGGTKLEIKRADAAPTVSIFPPSSEQLTSGGASVVCFLNNFYPKDINVKWKI
DGSERQNGVLNSWTDQDSKDSTYSMSSTLTLTKDEYERHNSYTCEATHKTSTSPIVKSENRNEC
SEQ ID NO: 68
BioE2440 VL nucleic acid sequence
GACATCGTGATGACACAGTCTCAGAAGTTCATGAGCACAAGCGTGGGCGACAGAGTGAGCGTGACCTGCAAGGCA
TCTCAGAACGTGGGCACCTACGTGGCCTGGTACGAGCAGAAGCTGGGGCAGAGCCCCAAGGCCCTGATCTTCAGC
GCAAGCTACAGATATACCGGCGTGCCCGACAGATTCACCGGCAGCGGCAGCGGCACCGACTTCACCCTGACCATC
AGCAACGTGCAGAGCGAGGACCTGGCCGAGTACTTCTGTCAGCAGTACGACAGCTACCCCCTGACCTTCGGCGGC
GGCACCAAGCTCGAGATCAAGCGCGCAGATGCTGCTCCTACCGTGAGCATCTTCCCGCCGTCCAGCGAACAACTC
ACTAGCGGAGGCGCGTCAGTGGTCTGCTTCCTTAACAATTTCTACCCTAAGGACATCAACGTCAAGTGGAAGATT
GACGGATCGGAACGCCAGAACGGAGTGCTGAACTCATGGACTGATCAGGATTCCAAAGACTCGACTTACTCCATG
TCCAGCACCCTGACCCTGACCAAAGACGAGTACGAAAGGCACAACTCGTACACGTGCGAAGCCACCCACAAGACT
TCCACCTCGCCCATCGTGAAGTCCTTCAATCGCAATGAGTGC
SEQ ID NO: 69
BioE2450 VH amino acid sequence
QVQLVESGGGVVQPGRSLRLSCAASGFTFSSYTMHWVRQAPGKGLEWVTFISYDGNNKYYADSVKGRFTISRDNS
KNTLYLQMNSLRAEDTAIYYCARTGWLGPFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKKVEPKSCDKTH
TCPPCPAPEAAGGPSVFLFPPKPKDTLMISRTPEVTCVVVDVSHEDPEVKENWYVDGVEVHNAKTKPREEQYNST
YRVVSVLTVLHQDWLNGKEYKCKVSNKALGAPIEKTISKAKGQPREPQVYTLPPSRDELTKNQVSLTCLVKGFYP
SDIAVEWESNGQPENNYKTTPPVLDSDGSFFLYSKLTVDKSRWQQGNVFSCSVMHEALHNHYTQKSLSLSPGK
SEQ ID NO: 70
BioE2450 VH nucleic acid sequence
CAAGTGCAGCTAGTTGAGTCTGGAGGCGGCGTGGTGCAGCCCGGCAGAAGCCTGAGACTGAGCTGCGCCGCAAGC
GGCTTCACCTTCAGCAGCTACACCATGCACTGGGTGAGACAAGCCCCCGGCAAGGGCCTGGAGTGGGTGACCTTC
ATCAGCTACGACGGCAACAACAAGTACTACGCCGACAGCGTGAAGGGCAGATTCACCATCAGCAGAGACAACAGC
AAGAACACCCTGTACCTGCAGATGAACAGCCTGAGAGCCGAGGACACCGCCATCTACTACTGCGCTAGAACCGGC
TGGCTGGGCCCCTTCGACTACTGGGGCCAAGGCACCCTGGTAACGGTAAGCAGCGCTAGTACCAAAGGGCCTAGC
GTGTTTCCTTTAGCCCCTAGCAGCAAAAGCACAAGCGGAGGCACCGCCGCCCTGGGCTGTCTGGTGAAGGATTAC
TTTCCTGAACCCGTGACTGTATCATGGAATAGCGGGGCCCTGACTAGCGGAGTGCATACCTTCCCCGCCGTGCTG
CAGAGCAGCGGCCTGTACAGCCTGAGCAGCGTGGTGACCGTGCCTAGCAGCAGCCTGGGCACACAGACCTACATC
TGCAACGTGAACCACAAGCCTAGCAACACCAAGGTGGACAAGAAGGTGGAGCCCAAGAGCTGTGACAAAACGCAC
ACCTGTCCGCCGTGCCCCGCCCCTGAGGCTGCCGGCGGCCCTAGCGTGTTCCTGTTTCCCCCCAAGCCCAAGGAC
ACCCTGATGATCAGCAGAACCCCTGAGGTGACCTGCGTGGTGGTGGACGTGAGCCACGAGGACCCTGAGGTGAAG
TTCAACTGGTACGTGGACGGCGTGGAGGTGCACAACGCCAAGACCAAGCCTAGAGAGGAGCAGTACAACAGCACC
TACAGAGTGGTGAGCGTGCTGACCGTGCTGCACCAAGACTGGCTGAACGGCAAGGAGTACAAGTGCAAGGTGAGC
AACAAGGCCCTGGGCGCCCCCATCGAGAAGACCATCAGCAAGGCCAAGGGGCAGCCTAGAGAGCCCCAAGTGTAC
ACCCTGCCCCCTAGCAGAGACGAGCTGACCAAGAACCAAGTGAGCCTGACCTGCCTAGTGAAGGGTTTCTACCCT
AGCGACATCGCCGTGGAGTGGGAGAGCAACGGGCAGCCTGAGAACAACTACAAGACCACCCCCCCCGTGCTGGAC
AGCGACGGCAGCTTCTTCCTGTACAGCAAGCTGACCGTGGACAAGAGCAGATGGCAGCAAGGCAACGTGTTCAGC
TGCAGCGTGATGCACGAGGCCCTGCACAACCACTACACACAGAAGAGCCTGAGCCTGAGCCCCGGCAAG
SEQ ID NO: 71
BioE2450 VL amino acid sequence
EIVLTQSPGTLSLSPGERATLSCRASQSVGSSYLAWYQQKPGQAPRLLIYGAFSRATGIPDRESGSGSGTDETLT
ISRLEPEDFAVYYCQQYGSSPWTFGQGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSENRGEC
SEQ ID NO: 72
BioE2450 VL nucleic acid sequence
GAGATCGTGCTGACACAGAGCCCCGGCACACTGTCTCTGAGCCCCGGCGAGAGAGCCACACTGAGCTGCAGAGCC
AGCCAGAGCGTGGGCAGCAGCTATCTGGCTTGGTACCAGCAGAAGCCCGGCCAAGCCCCTAGACTGCTGATTTAC
GGAGCCTTTAGCAGAGCCACCGGCATCCCCGACAGATTCAGCGGAAGCGGCAGCGGCACCGATTTCACACTGACA
ATCTCTAGACTGGAGCCAGAGGACTTCGCCGTGTACTACTGCCAGCAGTACGGCAGCAGCCCTTGGACCTTTGGC
CAAGGCACCAAGGTGGAGATCAAGATCAAGAGAACTGTGGCCGCGCCGTCAGTGTTTATCTTCCCTCCATCGGAT
GAACAGCTTAAGTCCGGCACGGCGTCTGTGGTCTGCCTGCTCAATAACTTTTACCCTAGGGAAGCTAAAGTCCAA
TGGAAAGTGGATAACGCCCTGCAGTCAGGAAACAGCCAGGAATCGGTTACCGAACAGGACAGCAAGGACAGCACT
TACTCCTTGTCGTCGACTCTTACTCTGAGCAAGGCCGATTACGAGAAGCACAAGGTCTACGCCTGCGAGGTCACC
CATCAGGGACTCTCGTCCCCGGTGACCAAATCCTTCAATAGAGGCGAATGC
*Included in Table 3 are RNA nucleic acid molecules (e.g., thymidines replaced with uridines), nucleic acid molecules encoding orthologs of the encoded proteins, as well as DNA, cDNA, or RNA nucleic acid sequences comprising a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any SEQ ID NO listed in Table 3, or a portion thereof. Such nucleic acid molecules can have a function of the full-length nucleic acid.
*Included in Table 3 are proteins, as well as polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any SEQ ID NO listed in Table 3, or a portion thereof. Such polypeptides can have a function of the full-length polypeptide.
*The polypeptide molecules or proteins of the present disclosure may further comprise an optional tag (e.g., His tag, etc.) and/or a leader sequence. An exemplary leader sequence may comprise the following sequence: MDFQVQIFSFLLISASVILSRG (SEQ ID NO: 55)
```

TABLE 4
Exemplary antigen-binding proteins that bind CTLA4

| Name | Construct |
|---|---|
| BioE2012 | Half antibody-ScFv |
| | IPI Fab Fc 121 ScFv [VL121 (G4S)3 VH 121] |
| BioE2021 | Ipi scFv |
| | [VL Ipi]-(Gly4Ser)3-[VH Ipi]-His10 tag |
| BioE2022 | Ipi F(ab′)2. |
| | BioE2022 also refers to the VH and CH1 domains of ipi that |
| | form ipi F(ab′)2 with BioE2023. See FIGS. 10D and 10H. |
| BioE2023 | Ipi VL and CL domains |
| BioE2031 | 121 scFv |
| | [VL 121]-(Gly4Ser)3-[VH 121]-His10 tag |
| BioE2032 | 121 HuIgG1 |
| BioE2033 | 121 F(ab′)2. |
| | BioE2033 also refers to the VH and CH1 domains of 121 that |
| | form 121 F(ab′)2 with BioE2034. See FIGS. 10F and 10I. |
| BioE2034 | 121 VL and CL domains |
| BioE2041 | Tandem Biparatopic ScFv (TBS); self-dimerizes |
| | VLIPI (G4S)3 VHIPI (KEA)6 VL121 (G4S)3 VH121 His6 tag |
| BioE2042 | Tandem Biparatopic ScFv (TBS); self-dimerizes |
| | VL121 (G4S)3 VH 121 (KEA)6 VLIPI (G4S)3 VH IPI His6 tag |
| BioE2051 | Anti-CTLA4 diabody (single-chain diabody; self-dimerizes; |
| | Tandem Diabody (TandAb)) comprising: |
| | [VH 121]-(GlyGlySer)3-[VL Ipi]-(GlyGlySer)3-[VH Ipi]- |
| | (GlyGlySer)3-[VL121]-His10 Tag |
| | (See FIG. 10A and FIG. 10C) |
| BioE2052 | Anti-CTLA4 diabody (single-chain diabody; self-dimerizes; |
| | Tandem Diabody (TandAb)) comprising: |
| | [VH Ipi]-(GlyGlySer)3-[VL 121]-(GlyGlySer)3-[VH 121]- |
| | (GlyGlySer)3-[VL Ipi]-His10 Tag |
| | (see FIG. 10A and FIG. 10B) |
| BioE2061 | Tandem Fab |
| | VH121--CH -(G4S)3 --VH IPI-CH-His tag (VL 121--CL/VL IPI--CL) |
| | BioE2061 comprises 3 polypeptides with the sequence of |
| | SEQ ID NOs: 31-33. See Wu et al. (2015) MAbs 7: 470-82 |
| BioE2062 | Tandem Fab |
| | VH IPI-CH-(G4S)3-VH 121--CH-His tag (VL IPI--CL/VL 121--CL) |
| | BioE2062 comprises 3 polypeptides with the sequence of |
| | SEQ ID NOs: 34-36. See Wu et al. (2015) MAbs 7: 470-82 |
| BioE2081 | Heterodimeric Fab/ScFv-Fc |
| | 121ScFv Fc IPI Fab Fc (Fc silent) KiH Fc |
| | BioE2081 comprises 3 polypeptides with the sequence of |
| | SEQ ID NOs: 47-49. BioE2081 contains the Fc-silent |
| | LALAPG mutation. |
| | *KiH stands for Knob in Hole. See e.g., Ridgway et al. |
| | (1996) Protein Engineering 9: 617-621 |
| BioE2082 | Heterodimeric Fab/ScFv-Fc |
| | 121 Fab Fc IPI ScFv Fc (Fc silent) KiH Fc |
| | BioE2082 comprises 3 polypeptides with the sequence of |
| | SEQ ID NOs: 50-52. BioE2082 contains the Fc-silent |
| | LALAPG mutation. |
| | *KiH stands for Knob in Hole. See e.g., Ridgway et al. |
| | (1996) Protein Engineering 9: 617-621 |
| BioE2091 | Tandem Fab |
| | VL IPI CL (G4S)3 VH 121 CH1 His tag (VH IPI CH1/VL 121 CL) |
| | BioE2091 comprises 3 polypeptides with the sequence of |
| | SEQ ID NOs: 37-39. See Wu et al. (2015) MAbs 7: 470-82 |
| BioE2092 | Tandem Fab |
| | VL 121 CL (G4S)3 VH IPI CH1 His tag (VH 121 CH1/VL IPI CL) |
| | BioE2092 comprises 3 polypeptides with the sequence of |
| | SEQ ID NOs: 40-42. See Wu et al. (2015) MAbs 7: 470-82 |
| BioE2111 | DART |
| | BioE2111 comprises 2 polypeptides with the sequence of |
| | SEQ ID NOs: 43-44. |
| BioE2201 | Ipi Fab′. BioE2201 also refers to the VH and CH1 domains |
| | of ipi that form ipi Fab′ with BioE2023. |
| | See FIGS. 10E and 10H. |
| BioE2202 | 121 Fab′. BioE2202 also refers to the VH and CH1 domains |
| | of 121 that form 121 Fab′ with BioE2034. |
| | See FIGS. 10G and 10I. |
| BioE2121 | Heterodimeric scFvs |
| | IPI scFv-Fc/121 scFv-Fc (KiH) |
| | BioE2121 comprises 2 polypeptides with the sequence of |
| | SEQ ID NOs: 53-54. BioE2121 contains the Fc-silent |
| | LALAPG mutation. |
| | *KiH stands for Knob in Hole. See e.g., Ridgway et al. |
| | (1996) Protein Engineering 9: 617-621 |

*Ipi: ipilimumab (see U.S. Pat. No. 6,984,720 B1)
*121: the humanized antibody disclosed in U.S. Pat. No. 7,034,121 B2

### Nucleic Acids and Vectors

A further object of the invention relates to nucleic acid sequences encoding monoclonal antibodies and fragments thereof, immunoglobulins, and polypeptides of the present invention.

For example, in certain embodiments, the present invention relates, in part, to a nucleic acid sequence encoding the VH domain of mAbs 9A3, 14C3, 8H3, or 4A3; or the VL domain of mAbs 9A3, 14C3, 8H3, or 4A3.

Typically, said nucleic acid is a DNA or RNA molecule, which may be included in any suitable vector, such as a plasmid, cosmid, episome, artificial chromosome, phage or a viral vector.

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. Thus, a further object of the invention relates to a vector comprising a nucleic acid of the present invention.

Such vectors may comprise regulatory elements, such as a promoter, enhancer, terminator and the like, to cause or direct expression of said polypeptide upon administration to a subject. Examples of promoters and enhancers used in the expression vector for animal cell include early promoter and enhancer of SV40 (Mizukami T. et al. 1987), LTR promoter and enhancer of Moloney mouse leukemia virus (Kuwana Y et al. 1987), promoter (Mason J O et al. 1985) and enhancer (Gillies S D et al. 1983) of immunoglobulin H chain and the like.

Any expression vector for animal cell can be used. Examples of suitable vectors include pAGE107 (Miyaji H et al. 1990), pAGE103 (Mizukami T et al. 1987), pHSG274 (Brady G et al. 1984), pKCR (O'Hare K et al. 1981), pSG1 beta d2-4-(Miyaji H et al. 1990) and the like. Other representative examples of plasmids include replicating plasmids comprising an origin of replication, or integrative plasmids, such as for instance pUC, pcDNA, pBR, and the like. Representative examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv-positive cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

Accordingly, the nucleic acids of the present disclosure in some aspects are incorporated into a vector. In this regard, the present disclosure provides vectors comprising any of the presently disclosed nucleic acids. In various aspects, the vector is a recombinant expression vector. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the present disclosure are not naturally-occurring as a whole. However, parts of the vectors can be naturally-occurring. The presently disclosed vectors can comprise any type of nucleotides, including, but not limited to DNA and RNA, which can be single-stranded or double-stranded, synthesized or obtained in part from natural sources, and which can contain natural, non-natural or altered nucleotides. The vectors can comprise naturally-occurring or non-naturally-occurring internucleotide linkages, or both types of linkages. In some aspects, the altered nucleotides or non-naturally occurring internucleotide linkages do not hinder the transcription or replication of the vector.

The vector of the present disclosure can be any suitable vector, and can be used to transduce, transform or transfect any suitable host. Suitable vectors include those designed for propagation and expansion or for expression or both, such as plasmids and viruses. The vector can be a plasmid based expression vector. In various aspects, the vector is selected from the group consisting of the pUC series (Fermentas Life Sciences), the pBluescript series (Stratagene, LaJolla, CA), the pET series (Novagen, Madison, WI), the pGEX series (Pharmacia Biotech, Uppsala, Sweden), and the pEX series (Clontech, Palo Alto, CA). Bacteriophage vectors, such as λGTIO, λGTl 1, λZapII (Stratagene), λEMBL4, and λNMl 149, also can be used. Examples of plant expression vectors include pBIOl, pBI101.2, pBI101.3, pBI121 and pBIN19 (Clontech). Examples of animal expression vectors include pEUK-Cl, pMAM and pMAMneo (Clontech). In some aspects, the vector is a viral vector, e.g., a retroviral vector. In various aspects, the vector is an adenovirus vector, an adeno-associated virus (AAV) vector, a Herpes Simplex Virus (HSV) vector, a Vesicular stomatitis virus (VSV) vector, vaccinia virus vector, or lentivirus vector. See, e.g., Howarth et al., Cell Biol. Toxicol. 26 (1): 1-20 (2010). In various aspects, the vector is a baculovirus vector which infects arthropods, e.g., insects. In various aspects, the baculovirus vector is an Autographacalifornica multiple nuclear virus (AcMNPV) or a Bombyxmorinuclear polyhedrosis (BmNPV). See, e.g., Khan, Adv Pharm Bull 3 (2): 257-263 (2013); Miller, Bioessays 11 (4): 91-96 (1989); Atkinson et al., Pestic Sci 28:215-224 (1990).

The vectors of the present disclosure can be prepared using standard recombinant DNA techniques described in, for example, Sambrook et al., supra, and Ausubel et al., supra. Constructs of expression vectors, which are circular or linear, can be prepared to contain a replication system functional in a prokaryotic or eukaryotic host cell. Replication systems can be derived, e.g., from CoIEl, 2μ plasmid, λ, SV40, bovine papilloma virus, and the like. In some aspects, the vector comprises regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA-based.

The vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the presently disclosed expression vectors include, for instance, neomycin/G418 resistance genes, hygromycin resistance genes, histidinol resistance genes, tetracycline resistance genes, and ampicillin resistance genes.

The vector can comprise a native or normative promoter operably linked to the nucleotide sequence encoding the polypeptide (including functional portions and functional variants thereof), or to the nucleotide sequence which is complementary to or which hybridizes to the nucleotide sequence encoding the polypeptide. The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, and a promoter found in the long-terminal repeat of the murine stem cell virus.

In another aspect, the present invention provides isolated nucleic acids that hybridize under selective hybridization conditions to a polynucleotide disclosed herein. Thus, the polynucleotides of this embodiment can be used for isolating, detecting, and/or quantifying nucleic acids comprising such polynucleotides. For example, polynucleotides of the present invention can be used to identify, isolate, or amplify partial or full-length clones in a deposited library. In some embodiments, the polynucleotides are genomic or cDNA sequences isolated, or otherwise complementary to, a cDNA from a human or mammalian nucleic acid library. Preferably, the cDNA library comprises at least 80% full-length sequences, preferably, at least 85% or 90% full-length sequences, and, preferably, at least 95% full-length sequences. The cDNA libraries can be normalized to increase the representation of rare sequences. Low or moderate stringency hybridization conditions are typically, but not exclusively, employed with sequences having a reduced sequence identity relative to complementary sequences. Moderate and high stringency conditions can optionally be employed for sequences of greater identity. Low stringency conditions allow selective hybridization of sequences having about 70% sequence identity and can be employed to identify orthologous or paralogous sequences. Optionally, polynucleotides of this invention will encode at least a portion of an antibody encoded by the polynucleotides described herein. The polynucleotides of this invention embrace nucleic acid sequences that can be employed for selective hybridization to a polynucleotide encoding an antibody of the present invention. See, e.g., Ausubel, supra; Colligan, supra, each entirely incorporated herein by reference.

### Host Cells

Provided herein are host cells comprising a nucleic acid or vector of the present disclosure. A further object of the present invention relates to a cell which has been transfected, infected or transformed by a nucleic acid and/or a vector according to the invention. The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed.”

The nucleic acids of the present invention may be used to produce a recombinant polypeptide of the invention in a suitable expression system. The term “expression system” means a host cell and compatible vector under suitable conditions, e.g. for the expression of a protein coded for by foreign DNA carried by the vector and introduced to the host cell.

Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene (hereinafter referred to as “DHFR gene”) is defective (Urlaub G et al; 1980), rat YB2/3HL.P2.G11.16Ag.20 cell (ATCC CRL 1662, hereinafter referred to as “YB2/0 cell”), and the like. The YB2/0 cell is preferred, since ADCC activity of chimeric or humanized antibodies is enhanced when expressed in this cell.

The present invention also relates to a method of producing a recombinant host cell expressing an antibody or a polypeptide of the invention according to the invention, said method comprising the steps consisting of (i) introducing in vitro or ex vivo a recombinant nucleic acid or a vector as described herein into a competent host cell, (ii) culturing in vitro or ex vivo the recombinant host cell obtained and (iii), optionally, selecting the cells which express and/or secrete said antibody or polypeptide. Such recombinant host cells can be used for the production of antibodies and polypeptides of the invention.

As used herein, the term “host cell” refers to any type of cell that can contain the presently disclosed vector and is capable of producing an expression product encoded by the nucleic acid (e.g., mRNA, protein). The host cell in some aspects is an adherent cell or a suspended cell, i.e., a cell that grows in suspension. The host cell in various aspects is a cultured cell or a primary cell, i.e., isolated directly from an organism, e.g., a human. The host cell can be of any cell type, can originate from any type of tissue, and can be of any developmental stage.

In certain aspects, the antigen-binding protein is a glycosylated protein and the host cell is a glycosylation-competent cell. In various aspects, the glycosylation-competent cell is an eukaryotic cell, including, but not limited to, a yeast cell, filamentous fungi cell, protozoa cell, algae cell, insect cell, or mammalian cell. Such host cells are described in the art. See, e.g., Frenzel, et al., Front Immunol 4:217 (2013). In various aspects, the eukaryotic cells are mammalian cells. In various aspects, the mammalian cells are non-human mammalian cells. In some aspects, the cells are Chinese Hamster Ovary (CHO) cells and derivatives thereof (e.g., CHO-K1, CHO pro-3), mouse myeloma cells (e.g., NS0, GS-NS0, Sp2/0), cells engineered to be deficient in dihydrofolatereductase (DHFR) activity (e.g., DUKX-X11, DG44), human embryonic kidney 293 (HEK293) cells or derivatives thereof (e.g., HEK293T, HEK293-EBNA), green African monkey kidney cells (e.g., COS cells, VERO cells), human cervical cancer cells (e.g., HeLa), human bone osteosarcoma epithelial cells U2-OS, adenocarcinoma human alveolar basal epithelial cells A549, human fibrosarcoma cells HT1080, mouse brain tumor cells CAD, embryonic carcinoma cells P19, mouse embryo fibroblast cells NIH 3T3, mouse fibroblast cells L929, mouse neuroblastoma cells N2a, human breast cancer cells MCF-7, retinoblastoma cells Y79, human retinoblastoma cells SO-Rb50, human liver cancer cells Hep G2, mouse B myeloma cells J558L, or baby hamster kidney (BHK) cells (Gaillet et al. 2007; Khan, Adv Pharm Bull 3 (2): 257-263 (2013)).

For purposes of amplifying or replicating the vector, the host cell is in some aspects is a prokaryotic cell, e.g., a bacterial cell.

Also provided by the present disclosure is a population of cells comprising at least one host cell described herein. The population of cells in some aspects is a heterogeneous population comprising the host cell comprising vectors described, in addition to at least one other cell, which does not comprise any of the vectors. Alternatively, in some aspects, the population of cells is a substantially homogeneous population, in which the population comprises mainly host cells (e.g., consisting essentially of) comprising the vector. The population in some aspects is a clonal population of cells, in which all cells of the population are clones of a single host cell comprising a vector, such that all cells of the population comprise the vector. In various embodiments of the present disclosure, the population of cells is a clonal population comprising host cells comprising a vector as described herein.

In certain aspects the host cell is a human cell that is autologous or allogeneic to the subject. In some embodiments, a nucleic acid of the present invention is transduced via a viral vector or transformed in other suitable methods (e.g., electroporation, etc.). Such host cells are transferred (e.g., grafted, implanted, etc.) to the subject for a prolonged treatment of the disease or condition, e.g., cancer.

### Manufacturing Methods

Also provided herein are methods of producing an antigen-binding protein which binds to CTLA4. In various embodiments, the method comprises culturing a host cell comprising a nucleic acid comprising a nucleotide sequence encoding the antigen-binding protein as described herein in a cell culture medium and harvesting the antigen-binding protein from the cell culture medium. The host cell can be any of the host cells described herein. In various aspects, the host cell is selected from the group consisting of: CHO cells, NS0 cells, COS cells, VERO cells, and BHK cells. In various aspects, the step of culturing a host cell comprises culturing the host cell in a growth medium to support the growth and expansion of the host cell. In various aspects, the growth medium increases cell density, culture viability and productivity in a timely manner. In various aspects, the growth medium comprises amino acids, vitamins, inorganic salts, glucose, and serum as a source of growth factors, hormones, and attachment factors. In various aspects, the growth medium is a fully chemically defined media consisting of amino acids, vitamins, trace elements, inorganic salts, lipids and insulin or insulin-like growth factors. In addition to nutrients, the growth medium also helps maintain pH and osmolality. Several growth media are commercially available and are described in the art. See, e.g., Arora, “Cell Culture Media: A Review” Mater Methods 3:175 (2013).

In various aspects, the method comprises culturing the host cell in a feed medium. In various aspects, the method comprises culturing in a feed medium in a fed-batch mode. Methods of recombinant protein production are known in the art. See, e.g., Li et al., “Cell culture processes for monoclonal antibody production” MAbs 2 (5): 466-477 (2010).

The method making an antigen-binding protein can comprise one or more steps for purifying the protein from a cell culture or the supernatant thereof and preferably recovering the purified protein. In various aspects, the method comprises one or more chromatography steps, e.g., affinity chromatography (e.g., protein A affinity chromatography, nickel resin for Histidine (His) tags), ion exchange chromatography, hydrophobic interaction chromatography. In various aspects, the method comprises purifying the protein using a Protein A affinity chromatography resin.

In various embodiments, the method further comprises steps for formulating the purified protein, etc., thereby obtaining a formulation comprising the purified protein. Such steps are described in Formulation and Process Development Strategies for Manufacturing, eds. Jameel and Hershenson, John Wiley & Sons, Inc. (Hoboken, NJ), 2010.

In various aspects, the antigen-binding protein linked to a polypeptide and the antigen-binding protein is part of a fusion protein. Thus, the present disclosure further provides methods of producing a fusion protein comprising an antigen-binding protein which binds to CTLA4. In various embodiments, the method comprises culturing a host cell comprising a nucleic acid comprising a nucleotide sequence encoding the fusion protein as described herein in a cell culture medium and harvesting the fusion protein from the cell culture medium.

Accordingly, the engineered antigen-binding protein of the present invention may be produced by any technique known in the art, such as, without limitation, any chemical, biological, genetic or enzymatic technique, either alone or in combination.

Knowing the amino acid sequence of the desired sequence, one skilled in the art can readily produce said antibodies or polypeptides, by standard techniques for production of polypeptides. For instance, they can be synthesized using well-known solid phase method, preferably using a commercially available peptide synthesis apparatus (such as that made by Applied Biosystems, Foster City, Calif.) and following the manufacturer's instructions. Alternatively, antibodies and other polypeptides of the present invention can be synthesized by recombinant DNA techniques as is well-known in the art. For example, these fragments can be obtained as DNA expression products after incorporation of DNA sequences encoding the desired (poly) peptide into expression vectors and introduction of such vectors into suitable eukaryotic or prokaryotic hosts that will express the desired polypeptide, from which they can be later isolated using well-known techniques.

In particular, the present invention further relates to a method of producing an antibody or a polypeptide of the invention, which method comprises the steps consisting of: (i) culturing a transformed host cell according to the invention under conditions suitable to allow expression of said antibody or polypeptide; and (ii) recovering the expressed antibody or polypeptide.

Antibodies and other polypeptides of the present invention are suitably separated from the culture medium by conventional immunoglobulin purification procedures such as, for example, protein A-Sepharose, hydroxylapatite chromatography, gel electrophoresis, dialysis, affinity chromatography, ammonium sulfate or ethanol precipitation, acid extraction, anion or cation exchange chromatography, phosphocellulose chromatography, hydrophobic interaction chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, N.Y., (1997-2001), e.g., Chapters 1, 4, 6, 8, 9, 10, each entirely incorporated herein by reference.

Chimeric antibodies (e.g., mouse-human chimeras or non-rodent-human chimeras) of the present invention can be produced by obtaining nucleic sequences encoding VL and VH domains as previously described, constructing a human chimeric antibody expression vector by inserting them into an expression vector for animal cell having genes encoding human antibody CH and human antibody CL, and expressing the coding sequence by introducing the expression vector into an animal cell. The CH domain of a human chimeric antibody can be any region which belongs to human immunoglobulin, such as the IgG class or a subclass thereof, such as IgG1, IgG2, IgG3 and IgG4. Similarly, the CL of a human chimeric antibody can be any region which belongs to Ig, such as the kappa class or lambda class. The chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Patent Publication PCT/US86/02269; Akira et al. European Patent Application 184,187; Taniguchi, M. European Patent Application 171,496; Morrison et al. European Patent Application 173,494; Neuberger et al. PCT Application WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. 84:214-218; Nishimura et al. (1987) Cancer Res. 47:999-1005; Wood et al. (1985) Nature 314:446-449; Shaw et al. (1988) J. Natl. Cancer Inst. 80:1553-1559); Morrison, S. L. (1985) Science 229:1202-1207; Oi et al. (1986) Biotechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

In addition, humanized antibodies can be made according to standard protocols such as those disclosed in U.S. Pat. No. 5,565,332. In another embodiment, antibody chains or specific binding pair members can be produced by recombination between vectors comprising nucleic acid molecules encoding a fusion of a polypeptide chain of a specific binding pair member and a component of a replicable generic display package and vectors containing nucleic acid molecules encoding a second polypeptide chain of a single binding pair member using techniques known in the art, e.g., as described in U.S. Pat. Nos. 5,565,332, 5,871,907, or 5,733,743. Humanized antibodies of the present invention can be produced by obtaining nucleic acid sequences encoding CDR domains, as previously described, constructing a humanized antibody expression vector by inserting them into an expression vector for animal cell having genes encoding (i) a heavy chain constant region identical to that of a human antibody and (ii) a light chain constant region identical to that of a human antibody, and expressing the genes by introducing the expression vector into an animal cell.

The humanized antibody expression vector may be either of a type in which a gene encoding an antibody heavy chain and a gene encoding an antibody light chain exists on separate vectors or of a type in which both genes exist on the same vector (tandem type).

Methods for producing humanized antibodies based on conventional recombinant DNA and gene transfection techniques are well-known in the art (See, e.g., Riechmann L. et al. 1988; Neuberger M S. et al. 1985). Antibodies can be humanized using a variety of techniques known in the art including, for example, CDR-grafting (EP 239,400; PCT publication WO91/09967; U.S. Pat. Nos. 5,225,539; 5,530,101; and 5,585,089), veneering or resurfacing (EP 592,106; EP 519,596; Padlan E A (1991); Studnicka G M et al. (1994); Roguska M A. et al. (1994)), and chain shuffling (U.S. Pat. No. 5,565,332). The general recombinant DNA technology for preparation of such antibodies is also known (see European Patent Application EP 125023 and International Patent Application WO 96/02576).

In addition, methods for producing antibody fragments are well-known. For example, Fab fragments of the present invention can be obtained by treating an antibody which specifically reacts with a ganglioside with a protease such as papain. Also, Fabs can be produced by inserting DNA encoding Fabs of the antibody into a vector for prokaryotic expression system, or for eukaryotic expression system, and introducing the vector into a prokaryote or eukaryote (as appropriate) to express the Fabs.

Similarly, F(ab′)2 fragments of the present invention can be obtained treating an antibody which specifically reacts with a ganglioside with a protease, pepsin. Also, the F(ab′)2 fragment can be produced by binding Fab′ described below via a thioether bond or a disulfide bond.

Fab′ fragments of the present invention can be obtained treating F(ab′)2 which specifically reacts with a ganglioside with a reducing agent, dithiothreitol. Also, the Fab′ fragments can be produced by inserting DNA encoding a Fab′ fragment of the antibody into an expression vector for prokaryote, or an expression vector for eukaryote, and introducing the vector into a prokaryote or eukaryote (as appropriate) to perform its expression.

In addition, scFvs of the present invention can be produced by obtaining cDNA encoding the VH and VL domains as previously described, constructing DNA encoding scFv, inserting the DNA into an expression vector for prokaryote, or an expression vector for eukaryote, and then introducing the expression vector into a prokaryote or eukaryote (as appropriate) to express the scFv. To generate a humanized scFv fragment, a well-known technology called CDR grafting may be used, which involves selecting the complementary determining regions (CDRs) from a donor scFv fragment, and grafting them onto a human scFv fragment framework of known three dimensional structure (see, e.g., WO98/45322; WO 87/02671; U.S. Pat. Nos. 5,859,205; 5,585,089; 4,816,567; EP0173494).

The diabody molecules of the present invention can be produced using a variety of methods well known in the art, including de novo protein synthesis and recombinant expression of nucleic acids encoding the binding proteins. The desired nucleic acid sequences can be produced by recombinant methods or by solid-phase DNA synthesis. Exemplary methods of producing a diabody are known in the art (see e.g., U.S. Pat. Nos. 5,637,481A, 9,017,687B1, US20180194840A1).

### Modification of the Antigen-Binding Proteins

Amino acid sequence modification(s) of the antigen-binding proteins (e.g., antibody or fragments thereof, e.g., diabody, F(ab′)2), described herein are contemplated. For example, it may be desirable to improve the binding affinity and/or other biological properties of the (e.g., antibody or fragments thereof, e.g., diabody, F(ab′)2). It is known that when a humanized antibody is produced by simply grafting only CDRs in VH and VL of an antibody derived from a non-human animal in FRs of the VH and VL of a human antibody, the antigen binding activity is reduced in comparison with that of the original antibody derived from a non-human animal. It is considered that several amino acid residues of the VH and VL of the non-human antibody, not only in CDRs but also in FRs, are directly or indirectly associated with the antigen binding activity. Hence, substitution of these amino acid residues with different amino acid residues derived from FRs of the VH and VL of the human antibody would reduce binding activity and can be corrected by replacing the amino acids with amino acid residues of the original antibody derived from a non-human animal.

Modifications and changes may be made in the structure of the antibodies of the present invention, and in the DNA sequences encoding them, and still obtain a functional molecule that encodes an antibody and polypeptide with desirable characteristics. For example, certain amino acids may be substituted by other amino acids in a protein structure without appreciable loss of activity. Since the interactive capacity and nature of a protein define the protein's biological functional activity, certain amino acid substitutions can be made in a protein sequence, and, of course, in its DNA encoding sequence, while nevertheless obtaining a protein with like properties. It is thus contemplated that various changes may be made in the antibodies sequences of the invention, or corresponding DNA sequences that encode said polypeptides, without appreciable loss of their biological activity.

In one embodiment, amino acid changes may be achieved by changing codons in the DNA sequence to encode conservative substitutions based on conservation of the genetic code. Specifically, there is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code (see genetic code chart above).

As described above, an important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In making the changes in the amino sequences of polypeptide, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamate (−3.5); glutamine (−3.5); aspartate (<RTI 3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well-known to those of skill in the art and include: arginine and lysine; glutamate and aspartate; serine and threonine; glutamine and asparagine; and valine, leucine and isoleucine.

Another type of amino acid modification of the antibody of the invention may be useful for altering the original glycosylation pattern of the antibody to, for example, increase stability. By “altering” is meant deleting one or more carbohydrate moieties found in the antibody, and/or adding one or more glycosylation sites that are not present in the antibody. Glycosylation of antibodies is typically N-linked. “N-linked” refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. Addition of glycosylation sites to the antibody is conveniently accomplished by altering the amino acid sequence such that it contains one or more of the above-described tripeptide sequences (for N-linked glycosylation sites). Another type of covalent modification involves chemically or enzymatically coupling glycosides to the antibody. These procedures are advantageous in that they do not require production of the antibody in a host cell that has glycosylation capabilities for N- or O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. For example, such methods are described in WO87/05330.

Similarly, removal of any carbohydrate moieties present on the antibody may be accomplished chemically or enzymatically. Chemical deglycosylation requires exposure of the antibody to the compound trifluoromethanesulfonic acid, or an equivalent compound. This treatment results in the cleavage of most or all sugars except the linking sugar (N-acetylglucosamine or N-acetyl galactosamine), while leaving the antibody intact. Chemical deglycosylation is described by Sojahr H. et al. (1987) and by Edge, A S. et al. (1981). Enzymatic cleavage of carbohydrate moieties on antibodies can be achieved by the use of a variety of endo- and exo-glycosidases as described by Thotakura, N R. et al. (1987).

Other modifications can involve the formation of immunoconjugates. For example, in one type of covalent modification, antibodies or proteins are covalently linked to one of a variety of non proteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

Conjugation of antibodies or other proteins of the present invention with heterologous agents can be made using a variety of bifunctional protein coupling agents including but not limited to N-succinimidyl (2-pyridyldithio) propionate (SPDP), succinimidyl (N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6 diisocyanate), and bis-active fluorine compounds (such as 1,5-difluoro-2,4-dinitrobenzene). For example, carbon labeled 1-isothiocyanatobenzyl methyldiethylene triaminepentaacetic acid (MX-DTPA) is an exemplary chelating agent for conjugation of radionucleotide to the antibody (WO 94/11026).

In another aspect, the present invention features an antigen-binding protein (e.g., antibody or fragments thereof, e.g., diabody, F(ab′)2) that specifically bind CTLA4 conjugated to a moiety that allows detection in vivo or in vitro. Conjugated antigen-binding protein can be used to monitor its presence in blood or tissues as part of a clinical testing procedure. Examples of detectable moieties include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate (FITC), rhodamine, dichlorotriazinylamine fluorescein, dansyl chloride or phycoerythrin (PE); an example of a luminescent material includes luminol; examples of bioluminescent materials include luciferase, luciferin, and aequorin, and examples of suitable radioactive material include ¹²⁵I, ¹³¹I, ³⁵S, or ³H. As used herein, the term “labeled”, with regard to the antibody, is intended to encompass direct labeling of the antibody by coupling (i.e., physically linking) a detectable substance, such as a radioactive agent or a fluorophore (e.g. fluorescein isothiocyanate (FITC) or phycoerythrin (PE) or indocyanine (Cy5)) to the antibody, as well as indirect labeling of the antibody by reactivity with a detectable substance. For example, an antibody may be labeled with a nucleic acid sequence that may be amplified and detected, or an antisense oligonucleotide to reduce expression of a particular gene, such that expression can then be detected and measured.

Techniques for conjugating such therapeutic moiety to an antigen-binding protein (e.g., antibody or fragments thereof) are well-known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243 56 (Alan R. Liss, Inc. 1985); Hellstrom et al., “Antibodies For Drug Delivery”, in Controlled Drug Delivery (2nd Ed.), Robinson et al. (eds.), pp. 623 53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475 506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303 16 (Academic Press 1985), and Thorpe et al., “The Preparation And Cytotoxic Properties Of Antibody-Toxin Conjugates”, Immunol. Rev., 62:119 58 (1982).

### Conjugates

The present disclosure also provides antigen-binding proteins attached, linked or conjugated to a second moiety (e.g., a heterologous moiety, a conjugate moiety). Accordingly, the present disclosure provides a conjugate comprising an antigen-binding protein and a heterologous moiety. As used herein, the term “heterologous moiety” is synonymous with “conjugate moiety” and refers to any molecule (chemical or biochemical, naturally-occurring or non-coded) which is different from the antigen-binding proteins of the present disclosure. Various heterologous moieties include, but are not limited to, a polymer, a carbohydrate, a lipid, a nucleic acid, an oligonucleotide, a DNA or RNA, an amino acid, peptide, polypeptide, protein, therapeutic agent, (e.g., a cytotoxic agent, cytokine), or a diagnostic agent.

In some embodiments, the heterologous moiety is a polymer. The polymer can be branched or unbranched. The polymer can be of any molecular weight. The polymer in some embodiments has an average molecular weight of between about 2 kDa to about 100 kDa (the term “about” indicating that in preparations of a water soluble polymer, some molecules will weigh more, some less, than the stated molecular weight). The average molecular weight of the polymer is in some aspect between about 5 kDa and about 50 kDa, between about 12 kDa to about 40 kDa or between about 20 kDa to about 35 kDa.

In some embodiments, the polymer is modified to have a single reactive group, such as an active ester for acylation or an aldehyde for alkylation, so that the degree of polymerization can be controlled. The polymer in some embodiments is water soluble so that the protein to which it is attached does not precipitate in an aqueous environment, such as a physiological environment. In some embodiments, when, for example, the composition is used for therapeutic use, the polymer is pharmaceutically acceptable. Additionally, in some aspects, the polymer is a mixture of polymers, e.g., a co-polymer, a block co-polymer.

In some embodiments, the polymer is selected from the group consisting of: polyamides, polycarbonates, polyalkylenes and derivatives thereof including, polyalkylene glycols, polyalkylene oxides, polyalkylene terepthalates, polymers of acrylic and methacrylic esters, including poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate), polyvinyl polymers including polyvinyl alcohols, polyvinyl ethers, polyvinyl esters, polyvinyl halides, poly(vinyl acetate), and polyvinylpyrrolidone, polyglycolides, polysiloxanes, polyurethanes and co-polymers thereof, celluloses including alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt, polypropylene, polyethylenes including poly(ethylene glycol), poly(ethylene oxide), and poly(ethylene terephthalate), and polystyrene.

A particularly preferred water-soluble polymer for use herein is polyethylene glycol (PEG). As used herein, polyethylene glycol is meant to encompass any of the forms of PEG that can be used to derivatize other proteins, such as mono-(C1-C10) alkoxy- or aryloxy-polyethylene glycol. PEG is a linear or branched neutral polyether, available in a broad range of molecular weights, and is soluble in water and most organic solvents.

In some embodiments, the heterologous moiety is a carbohydrate. In some embodiments, the carbohydrate is a monosaccharide (e.g., glucose, galactose, fructose), a disaccharide (e.g., sucrose, lactose, maltose), an oligosaccharide (e.g., raffinose, stachyose), a polysaccharide (a starch, amylase, amylopectin, cellulose, chitin, callose, laminarin, xylan, mannan, fucoidan, galactomannan.

In some embodiments, the heterologous moiety is a lipid. The lipid, in some embodiments, is a fatty acid, eicosanoid, prostaglandin, leukotriene, thromboxane, N-acyl ethanolamine), glycerolipid (e.g., mono-, di-, tri-substituted glycerols), glycerophospholipid (e.g., phosphatidylcholine, phosphatidylinositol, phosphatidylethanolamine, phosphatidylserine), sphingolipid (e.g., sphingosine, ceramide), sterol lipid (e.g., steroid, cholesterol), prenol lipid, saccharolipid, or a polyketide, oil, wax, cholesterol, sterol, fat-soluble vitamin, monoglyceride, diglyceride, triglyceride, a phospholipid.

In some embodiments, the heterologous moiety is a therapeutic agent. The therapeutic agent can be any of those known in the art. Examples of therapeutic agents that are contemplated herein include, but are not limited to, natural enzymes, proteins derived from natural sources, recombinant proteins, natural peptides, synthetic peptides, cyclic peptides, antibodies, receptor agonists, cytotoxic agents, immunoglobins, beta-adrenergic blocking agents, calcium channel blockers, coronary vasodilators, cardiac glycosides, antiarrhythmics, cardiac sympathomemetics, angiotensin converting enzyme (ACE) inhibitors, diuretics, inotropes, cholesterol and triglyceride reducers, bile acid sequestrants, fibrates, 3-hydroxy-3-methylgluteryl (HMG)-CoA reductase inhibitors, niacin derivatives, antiadrenergic agents, alpha-adrenergic blocking agents, centrally acting antiadrenergic agents, vasodilators, potassium-sparing agents, thiazides and related agents, angiotensin II receptor antagonists, peripheral vasodilators, antiandrogens, estrogens, antibiotics, retinoids, insulins and analogs, alpha-glucosidase inhibitors, biguanides, meglitinides, sulfonylureas, thizaolidinediones, androgens, progestogens, bone metabolism regulators, anterior pituitary hormones, hypothalamic hormones, posterior pituitary hormones, gonadotropins, gonadotropin-releasing hormone antagonists, ovulation stimulants, selective estrogen receptor modulators, antithyroid agents, thyroid hormones, bulk forming agents, laxatives, antiperistaltics, flora modifiers, intestinal adsorbents, intestinal anti-infectives, antianorexic, anticachexic, antibulimics, appetite suppressants, antiobesity agents, antacids, upper gastrointestinal tract agents, anticholinergic agents, aminosalicylic acid derivatives, biological response modifiers, corticosteroids, antispasmodics, 5-HT4 partial agonists, antihistamines, cannabinoids, dopamine antagonists, serotonin antagonists, cytoprotectives, histamine H2-receptor antagonists, mucosal protective agent, proton pump inhibitors, H. pylori eradication therapy, erythropoieses stimulants, hematopoietic agents, anemia agents, heparins, antifibrinolytics, hemostatics, blood coagulation factors, adenosine diphosphate inhibitors, glycoprotein receptor inhibitors, fibrinogen-platelet binding inhibitors, thromboxane-A2 inhibitors, plasminogen activators, antithrombotic agents, glucocorticoids, mineralcorticoids, corticosteroids, selective immunosuppressive agents, antifungals, drugs involved in prophylactic therapy, AIDS-associated infections, cytomegalovirus, non-nucleoside reverse transcriptase inhibitors, nucleoside analog reverse transcriptse inhibitors, protease inhibitors, anemia, Kaposi's sarcoma, aminoglycosides, carbapenems, cephalosporins, glycopoptides, lincosamides, macrolies, oxazolidinones, penicillins, streptogramins, sulfonamides, trimethoprim and derivatives, tetracyclines, anthelmintics, amebicies, biguanides, cinchona alkaloids, folic acid antagonists, quinoline derivatives, Pneumocystis carinii therapy, hydrazides, imidazoles, triazoles, nitroimidzaoles, cyclic amines, neuraminidase inhibitors, nucleosides, phosphate binders, cholinesterase inhibitors, adjunctive therapy, barbiturates and derivatives, benzodiazepines, gamma aminobutyric acid derivatives, hydantoin derivatives, iminostilbene derivatives, succinimide derivatives, anticonvulsants, ergot alkaloids, antimigrane preparations, biological response modifiers, carbamic acid eaters, tricyclic derivatives, depolarizing agents, nondepolarizing agents, neuromuscular paralytic agents, CNS stimulants, dopaminergic reagents, monoamine oxidase inhibitors, COMT inhibitors, alkyl sulphonates, ethylenimines, imidazotetrazines, nitrogen mustard analogs, nitrosoureas, platinum-containing compounds, antimetabolites, purine analogs, pyrimidine analogs, urea derivatives, antracyclines, actinomycinds, camptothecin derivatives, epipodophyllotoxins, taxanes, vinca alkaloids and analogs, antiandrogens, antiestrogens, nonsteroidal aromatase inhibitors, protein kinase inhibitor antineoplastics, azaspirodecanedione derivatives, anxiolytics, stimulants, monoamind reuptake inhibitors, selective serotonin reuptake inhibitors, antidepressants, benzisooxazole derivatives, butyrophenone derivatives, dibenzodiazepine derivatives, dibenzothiazepine derivatives, diphenylbutylpiperidine derivatives, phenothiazines, thienobenzodiazepine derivatives, thioxanthene derivatives, allergenic extracts, nonsteroidal agents, leukotriene receptor antagonists, xanthines, endothelin receptor antagonist, prostaglandins, lung surfactants, mucolytics, antimitotics, uricosurics, xanthine oxidase inhibitors, phosphodiesterase inhibitors, metheamine salts, nitrofuran derivatives, quinolones, smooth muscle relaxants, parasympathomimetic agents, halogenated hydrocarbons, esters of amino benzoic acid, amides (e.g. lidocaine, articaine hydrochloride, bupivacaine hydrochloride), antipyretics, hynotics and sedatives, cyclopyrrolones, pyrazolopyrimidines, nonsteroidal anti-inflammatory drugs, opioids, para-aminophenol derivatives, alcohol dehydrogenase inhibitor, heparin antagonists, adsorbents, emetics, opoid antagonists, cholinesterase reactivators, nicotine replacement therapy, vitamin A analogs and antagonists, vitamin B analogs and antagonists, vitamin C analogs and antagonists, vitamin D analogs and antagonists, vitamin E analogs and antagonists, vitamin K analogs and antagonists.

The antigen-binding proteins of the present disclosure can be conjugated to one or more cytokines and growth factors that are effective in inhibiting tumor metastasis, and wherein the cytokine or growth factor has been shown to have an antiproliferative effect on at least one cell population. Such cytokines, lymphokines, growth factors, or other hematopoietic factors include, but are not limited to: M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IFN, TNFα, TNF1, TNF2, G-CSF, Meg-CSF, GM-CSF, thrombopoietin, stem cell factor, and erythropoietin. Additional growth factors for use herein include angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor α, cytokine-induced neutrophil chemotactic factor 1, cytokine-induced neutrophil, chemotactic factor 2 α, cytokine-induced neutrophil chemotactic factor 2 β, β endothelial cell growth factor, endothelin 1, epithelial-derived neutrophil attractant, glial cell line-derived neutrophic factor receptor α 1, glial cell line-derived neutrophic factor receptor α 2, growth related protein, growth related protein α, growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor nerve growth factor receptor, neurotrophin-3, neurotrophin-4, pre-B cell growth stimulating factor, stem cell factor, stem cell factor receptor, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor β binding protein III, tumor necrosis factor receptor type I, tumor necrosis factor receptor type II, urokinase-type plasminogen activator receptor, and chimeric proteins and biologically or immunologically active fragments thereof.

The present disclosure also provides conjugates comprising an antigen-binding protein of the present disclosure linked to a polypeptide, such that the conjugate is a fusion protein. Therefore, the present disclosure provides fusion proteins comprising an antigen-binding protein of the present disclosure linked to a polypeptide. In various embodiments, the polypeptide is a diagnostic label, e.g., a fluorescent protein, such as green fluorescent protein, or other tag, e.g., Myc tag. In various aspects, the polypeptide is one of the cytokines, lymphokines, growth factors, or other hematopoietic factors listed above.

### Compositions, Pharmaceutical Compositions, and Formulations

Compositions comprising an antigen-binding protein, a nucleic acid, a vector, a host cell, or a conjugate as presently disclosed are provided herein. The compositions in some aspects comprise the antigen-binding proteins in isolated and/or purified form. In some aspects, the composition comprises a single type (e.g., structure) of an antigen-binding protein of the present disclosure or comprises a combination of two or more antigen-binding proteins of the present disclosure, wherein the combination comprises two or more antigen-binding proteins of different types (e.g., structures).

In some aspects, the composition comprises agents which enhance the chemico-physico features of the antigen-binding protein, e.g., via stabilizing the antigen-binding protein at certain temperatures, e.g., room temperature, increasing shelf life, reducing degradation, e.g., oxidation protease mediated degradation, increasing half-life of the antigen-binding protein, etc. In some aspects, the composition comprises any of the agents disclosed herein as a heterologous moiety or conjugate moiety, optionally in admixture with the antigen-binding proteins of the present disclosure or conjugated to the antigen-binding proteins.

In various aspects of the present disclosure, the composition additionally comprises a pharmaceutically acceptable carrier, diluents, or excipient. In some embodiments, the antigen-binding protein, a nucleic acid, a vector, a host cell, or a conjugate as presently disclosed (hereinafter referred to as “active agents”) is formulated into a pharmaceutical composition comprising the active agent, along with a pharmaceutically acceptable carrier, diluent, or excipient. In this regard, the present disclosure further provides pharmaceutical compositions comprising an active agent which is intended for administration to a subject, e.g., a mammal.

In some embodiments, the active agent is present in the pharmaceutical composition at a purity level suitable for administration to a patient. In some embodiments, the active agent has a purity level of at least about 90%, about 91%, about 92%, about 93%, about 94%, about 95%, about 96%, about 97%, about 98% or about 99%, and a pharmaceutically acceptable diluent, carrier or excipient. In some embodiments, the compositions contain an active agent at a concentration of about 0.001 to about 30.0 mg/ml.

In various aspects, the pharmaceutical compositions comprise a pharmaceutically acceptable carrier. As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

The pharmaceutical composition can comprise any pharmaceutically acceptable ingredients, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, U K, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety.

In various aspects, the pharmaceutical composition comprises formulation materials that are nontoxic to recipients at the dosages and concentrations employed. In specific embodiments, pharmaceutical compositions comprising an active agent and one or more pharmaceutically acceptable salts; polyols; surfactants; osmotic balancing agents; tonicity agents; anti-oxidants; antibiotics; antimycotics; bulking agents; lyoprotectants; anti-foaming agents; chelating agents; preservatives; colorants; analgesics; or additional pharmaceutical agents. In various aspects, the pharmaceutical composition comprises one or more polyols and/or one or more surfactants, optionally, in addition to one or more excipients, including but not limited to, pharmaceutically acceptable salts; osmotic balancing agents (tonicity agents); anti-oxidants; antibiotics; antimycotics; bulking agents; lyoprotectants; anti-foaming agents; chelating agents; preservatives; colorants; and analgesics.

In certain embodiments, the pharmaceutical composition can contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, Remington's Pharmaceutical Sciences, 18″ Edition, (A. R. Genrmo, ed.), 1990, Mack Publishing Company.

The pharmaceutical compositions can be formulated to achieve a physiologically compatible pH. In some embodiments, the pH of the pharmaceutical composition can be for example between about 4 or about 5 and about 8.0 or about 4.5 and about 7.5 or about 5.0 to about 7.5. In various embodiments, the pH of the pharmaceutical composition is between 5.5 and 7.5.

The present disclosure provides methods of producing a pharmaceutical composition. In various aspects, the method comprises combining the antigen-binding protein, conjugate, fusion protein, nucleic acid, vector, host cell, or a combination thereof, with a pharmaceutically acceptable carrier, diluent, or excipient.

### Administration

With regard to the present disclosure, the active agent, or pharmaceutical composition comprising the same, can be administered to the subject via any suitable route of administration. For example, the active agent can be administered to a subject via parenteral, nasal, oral, pulmonary, topical, vaginal, or rectal administration. The following discussion on routes of administration is merely provided to illustrate various embodiments and should not be construed as limiting the scope in any way.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The term, “parenteral” means not through the alimentary canal but by some other route such as subcutaneous, intramuscular, intraspinal, or intravenous. The active agent of the present disclosure can be administered with a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-153-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and (e) mixtures thereof.

The parenteral formulations in some embodiments contain from about 0.5% to about 25% by weight of the active agent of the present disclosure in solution. Preservatives and buffers can be used. In order to minimize or eliminate irritation at the site of injection, such compositions can contain one or more nonionic surfactants having a hydrophile-lipophile balance (HLB) of from about 12 to about 17. The quantity of surfactant in such formulations will typically range from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations in some aspects are presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions in some aspects are prepared from sterile powders, granules, and tablets of the kind previously described.

Injectable formulations are in accordance with the present disclosure. The requirements for effective pharmaceutical carriers for injectable compositions are well-known to those of ordinary skill in the art (see, e.g., Pharmaceutics and Pharmacy Practice, J. B. Lippincott Company, Philadelphia, PA, Banker and Chalmers, eds., pages 238-250 (1982), and ASHP Handbook on Injectable Drugs, Toissel, 4th ed., pages 622-630 (1986)).

### Dosages

The active agents of the disclosure are believed to be useful in methods of inhibiting tumor growth, as well as other methods, as further described herein, including methods of treating or preventing cancer. For purposes of the disclosure, the amount or dose of the active agent administered should be sufficient to effect, e.g., a therapeutic or prophylactic response, in the subject or animal over a reasonable time frame. For example, the dose of the active agent of the present disclosure should be sufficient to treat cancer as described herein in a period of from about 1 to 4 minutes, 1 to 4 hours or 1 to 4 weeks or longer, e.g., 5 to 20 or more weeks, from the time of administration. In certain embodiments, the time period could be even longer. The dose will be determined by the efficacy of the particular active agent and the condition of the animal (e.g., human), as well as the body weight of the animal (e.g., human) to be treated.

Many assays for determining an administered dose are known in the art. For purposes herein, an assay, which comprises comparing the extent to which cancer is treated upon administration of a given dose of the active agent of the present disclosure to a mammal among a set of mammals, each set of which is given a different dose of the active agent, could be used to determine a starting dose to be administered to a mammal. The extent to which cancer is treated upon administration of a certain dose can be represented by, for example, the extent of tumor regression achieved with the active agent in a mouse xenograft model. Methods of assaying tumor regression are known in the art and described herein in the Examples.

The dose of the active agent of the present disclosure also will be determined by the existence, nature and extent of any adverse side effects that might accompany the administration of a particular active agent of the present disclosure. Typically, the attending physician will decide the dosage of the active agent of the present disclosure with which to treat each individual patient, taking into consideration a variety of factors, such as age, body weight, general health, diet, sex, active agent of the present disclosure to be administered, route of administration, and the severity of the condition being treated. By way of example and not intending to limit the present disclosure, the dose of the active agent of the present disclosure can be about 0.0001 to about 1 g/kg body weight of the subject being treated/day, from about 0.0001 to about 0.001 g/kg body weight/day, or about 0.01 mg to about 1 g/kg body weight/day.

### Controlled Release Formulations

In some embodiments, the active agents described herein can be modified into a depot form, such that the manner in which the active agent of the present disclosure is released into the body to which it is administered is controlled with respect to time and location within the body (see, for example, U.S. Pat. No. 4,450,150). Depot forms of active agents of the present disclosure can be, for example, an implantable composition comprising the active agents and a porous or non-porous material, such as a polymer, wherein the active agent is encapsulated by or diffused throughout the material and/or degradation of the non-porous material. The depot is then implanted into the desired location within the body of the subject and the active agent is released from the implant at a predetermined rate.

The pharmaceutical composition comprising the active agent in certain aspects is modified to have any type of in vivo release profile. In some aspects, the pharmaceutical composition is an immediate release, controlled release, sustained release, extended release, delayed release, or bi-phasic release formulation. Methods of formulating peptides for controlled release are known in the art. See, for example, Qian et al., J Pharm 374:46-52 (2009) and International Patent Application Publication Nos. WO 2008/130158; WO2004/033036; WO2000/032218; and WO 1999/040942.

The instant compositions can further comprise, for example, micelles or liposomes, or some other encapsulated form, or can be administered in an extended release form to provide a prolonged storage and/or delivery effect.

### Method of Preventing or Treating a Disease

The antigen-binding proteins of the present disclosure are useful for inhibiting tumor growth. Without being bound to a particular theory, the inhibiting action of the antigen-binding proteins provided herein allow such entities to be useful in methods of treating cancer.

In certain aspects, provided herein is a method of preventing or treating a subject afflicted with cancer, the method comprising administering to the subject at least one engineered antigen-binding protein of the present disclosure or a pharmaceutical composition comprising same.

In certain aspects, also provided herein is a method of reducing proliferation of a cancer cell in a subject in need thereof, the method comprising administering to the subject at least one engineered antigen-binding protein of the present disclosure or a pharmaceutical composition comprising same.

Further provided herein are methods of inhibiting tumor growth in a subject and methods of reducing tumor size in a subject. In various embodiments, the methods comprise administering to the subject the pharmaceutical composition of the present disclosure in an amount effective for inhibiting tumor growth or reducing tumor size in the subject.

In certain embodiments, the therapeutically effective amount of an engineered antigen-binding protein or pharmaceutical composition is administered to a subject in need thereof. In other embodiments, the cells that are autologous or allogeneic to the subject are obtained and transduced (e.g., via a viral vector, such as AAV) or otherwise transformed with a nucleic acid (or a vector comprising same) that encodes any one of the engineered antigen-binding protein of the present disclosure. In preferred embodiments, such nucleic acid is stably integrated into the host cell genome. Upon confirming transformation of the nucleic acid, the cells are introduced to the subject (e.g., grafted or implanted) to supply a continued source of the antigen-binding proteins (i.e., expressed by the grafted cells and secreted into blood).

In various aspects, the growth of melanoma (e.g., unresectable or metastatic melanoma), renal cell carcinoma (RCC), colorectal cancer, hepatocellular carcinoma, non-small cell lung cancer (NSCLC), malignant pleural mesothelioma, small cell lung cancer (SCLC), breast cancer, head and neck cancer, bladder cancer, urothelial carcinoma, Merkel cell cancer, cervical cancer, hepatocellular carcinoma, gastric cancer, cutaneous squamous cell cancer, Hodgkin's lymphoma, or B-cell lymphoma is inhibited. In various aspects, the size of melanoma (e.g., unresectable or metastatic melanoma), renal cell carcinoma (RCC), colorectal cancer, hepatocellular carcinoma, non-small cell lung cancer (NSCLC), malignant pleural mesothelioma, small cell lung cancer (SCLC), breast cancer, head and neck cancer, bladder cancer, urothelial carcinoma, Merkel cell cancer, cervical cancer, hepatocellular carcinoma, gastric cancer, cutaneous squamous cell cancer, Hodgkin's lymphoma, or B-cell lymphoma is reduced.

As used herein, the term “inhibit” or “reduce” and words stemming therefrom may not be a 100% or complete inhibition or reduction. Rather, there are varying degrees of inhibition or reduction of which one of ordinary skill in the art recognizes as having a potential benefit or therapeutic effect. In this respect, the antigen-binding proteins of the present disclosure may inhibit tumor growth or reduce tumor size to any amount or level. In various embodiments, the inhibition provided by the methods of the present disclosure is at least or about a 10% inhibition (e.g., at least or about a 20% inhibition, at least or about a 30% inhibition, at least or about a 40% inhibition, at least or about a 50% inhibition, at least or about a 60% inhibition, at least or about a 70% inhibition, at least or about a 80% inhibition, at least or about a 90% inhibition, at least or about a 95% inhibition, at least or about a 98% inhibition). In various embodiments, the reduction provided by the methods of the present disclosure is at least or about a 10% reduction (e.g., at least or about a 20% reduction, at least or about a 30% reduction, at least or about a 40% reduction, at least or about a 50% reduction, at least or about a 60% reduction, at least or about a 70% reduction, at least or about a 80% reduction, at least or about a 90% reduction, at least or about a 95% reduction, at least or about a 98% reduction).

As used herein, the term “treat,” as well as words related thereto, do not necessarily imply 100% or complete treatment. Rather, there are varying degrees of treatment of which one of ordinary skill in the art recognizes as having a potential benefit or therapeutic effect. In this respect, the methods of treating cancer of the present disclosure can provide any amount or any level of treatment. Furthermore, the treatment provided by the method of the present disclosure can include treatment of one or more conditions or symptoms or signs of the cancer being treated. Also, the treatment provided by the methods of the present disclosure can encompass slowing the progression of the cancer. For example, the methods can treat cancer by virtue of enhancing the T cell activity or an immune response against the cancer, reducing tumor or cancer growth, reducing metastasis of tumor cells, increasing cell death of tumor or cancer cells, and the like. In various aspects, the methods treat by way of delaying the onset or recurrence of the cancer by at least 1 day, 2 days, 4 days, 6 days, 8 days, 10 days, 15 days, 30 days, two months, 3 months, 4 months, 6 months, 1 year, 2 years, 3 years, 4 years, or more. In various aspects, the methods treat by way increasing the survival of the subject.

Numerous embodiments are further provided that can be applied to any aspect of the present invention and/or combined with any other embodiment described herein. For example, in some embodiments, the at least one engineered antigen-binding protein or the pharmaceutical composition (a) reduces the number of proliferating cancer cells in the cancer; (b) reduces the volume or size of a tumor of the cancer; (c) increases the immune response against the cancer; and/or (d) activates the T cell.

In some embodiments, the method further comprises administering to the subject an additional cancer therapy. In some embodiments, the additional cancer therapy is selected from the group consisting of immunotherapy, checkpoint blockade, cancer vaccines, chimeric antigen receptors, chemotherapy, radiation, target therapy, and surgery, optionally wherein the additional cancer therapy is nivolumab.

In certain aspects, provided herein is a method of increasing an immune response in a subject, the method comprising administering to the subject at least one engineered antigen-binding protein of the present disclosure or a pharmaceutical composition comprising same.

In certain aspects, provided herein is a method of activating a T cell, the method comprising contacting the T cells with at least one engineered antigen-binding protein of the present disclosure or a pharmaceutical composition comprising same. Such method may be used in vivo, in vitro, or ex vivo.

In certain aspects, provided herein is a method of preventing or treating a disease or a condition characterized by aberrant expression or activity of a CTLA4 protein in a subject in need thereof, the method comprising administering to the subject at least one engineered antigen-binding protein of the present disclosure or a pharmaceutical composition comprising same. In some embodiments, the disease or condition is a cancer, autoimmune disease, infection, or inflammatory disease.

### Cancer

Cancer, tumor, or hyperproliferative disorder refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells may exist alone within an animal, or may be a non-tumorigenic cancer cell, such as a leukemia cell. Cancers include, but are not limited to, B cell cancer, e.g., multiple myeloma, Waldenström's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma (SCLC), bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithlelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

In some embodiments, the cancer is selected from pancreatic cancer, lung cancer, non-small cell lung cancer (NSCLC), malignant pleural mesothelioma, small cell lung cancer (SCLC), renal cell carcinoma (RCC), breast cancer, liver cancer, hepatocellular carcinoma, kidney cancer, skin cancer, melanoma, thyroid cancer, gall bladder cancer, head-and-neck (squamous) cancer, stomach (gastric) cancer, head and neck cancer, bladder cancer, urothelial carcinoma, Merkel cell cancer, colon cancer, colorectal cancer, intestinal cancer, ovarian cancer, cervical cancer, testicular cancer, esophageal cancer, buccal cancer, brain cancer, blood cancers, lymphomas (B and T cell lymphomas), mesothelioma, cutaneous squamous cell cancer, Hodgkin's lymphoma, B-cell lymphoma, and a malignant or metastatic form thereof.

### Cancer Therapy

The therapeutic agents of the present invention can be used alone or can be administered in combination therapy with, e.g., chemotherapeutic agents, hormones, antiangiogens, radiolabelled, compounds, or with surgery, cryotherapy, immunotherapy, cancer vaccine, immune cell engineering (e.g., CAR-T), and/or radiotherapy. The preceding treatment methods can be administered in conjunction with other forms of conventional therapy (e.g., standard-of-care treatments for cancer well-known to the skilled artisan), either consecutively with, pre- or post-conventional therapy. For example, agents of the present invention can be administered with a therapeutically effective dose of chemotherapeutic agent. In other embodiments, agents of the present invention are administered in conjunction with chemotherapy to enhance the activity and efficacy of the chemotherapeutic agent. The Physicians' Desk Reference (PDR) discloses dosages of chemotherapeutic agents that have been used in the treatment of various cancers. The dosing regimen and dosages of these aforementioned chemotherapeutic drugs that are therapeutically effective will depend on the particular cancer being treated, the extent of the disease and other factors familiar to the physician of skill in the art, and can be determined by the physician.

Immunotherapy is a targeted therapy that may comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). For example, anti-VEGF is known to be effective in treating renal cell carcinoma. Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

Immunotherapy also encompasses immune checkpoint modulators. Immune checkpoints are a group of molecules on the cell surface of CD4+ and/or CD8+ T cells that fine-tune immune responses by down-modulating or inhibiting an anti-tumor immune response. Immune checkpoint proteins are well-known in the art and include, without limitation, CTLA4, PD-1, VISTA, B7-H2, B7-H3, PD-L1, B7-H4, B7-H6, 2B4, ICOS, HVEM, PD-L2, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, HHLA2, TMIDG2, KIR3DL3, and A2aR (see, for example, WO 2012/177624). Inhibition of one or more immune checkpoint inhibitors can block or otherwise neutralize inhibitory signaling to thereby upregulate an immune response in order to more efficaciously treat cancer. In some embodiments, the cancer therapy one or more inhibitors of immune checkpoints (immune checkpoint inhibition therapy), such as PD1, PD-L1, and/or CD47 inhibitors. In some embodiments, the cancer therapy is nivolumab.

Adoptive cell-based immunotherapies can be combined with the therapies of the present invention. Well-known adoptive cell-based immunotherapeutic modalities, including, without limitation, irradiated autologous or allogeneic tumor cells, tumor lysates or apoptotic tumor cells, antigen-presenting cell-based immunotherapy, dendritic cell-based immunotherapy, adoptive T cell transfer, adoptive CAR T cell therapy, autologous immune enhancement therapy (AIET), cancer vaccines, and/or antigen presenting cells. Such cell-based immunotherapies can be further modified to express one or more gene products to further modulate immune responses, such as expressing cytokines like GM-CSF, and/or to express tumor-associated antigen (TAA) antigens, such as Mage-1, gp-100, and the like.

The term “chimeric antigen receptor” or “CAR” refers to engineered T cell receptors (TCR) having a desired antigen specificity. T lymphocytes recognize specific antigens through interaction of the T cell receptor (TCR) with short peptides presented by major histocompatibility complex (MHC) class I or II molecules. For initial activation and clonal expansion, naive T cells are dependent on professional antigen-presenting cells (APCs) that provide additional co-stimulatory signals. TCR activation in the absence of co-stimulation can result in unresponsiveness and clonal anergy. To bypass immunization, different approaches for the derivation of cytotoxic effector cells with grafted recognition specificity have been developed. CARs have been constructed that consist of binding domains derived from natural ligands or antibodies specific for cell-surface components of the TCR-associated CD3 complex. Upon antigen binding, such chimeric antigen receptors link to endogenous signaling pathways in the effector cell and generate activating signals similar to those initiated by the TCR complex. Since the first reports on chimeric antigen receptors, this concept has steadily been refined and the molecular design of chimeric receptors has been optimized and routinely use any number of well-known binding domains, such as scFV and another protein binding fragments described herein.

In other embodiments, immunotherapy comprises non-cell-based immunotherapies. In some embodiments, compositions comprising antigens with or without vaccine-enhancing adjuvants are used. Such compositions exist in many well-known forms, such as peptide compositions, oncolytic viruses, recombinant antigen comprising fusion proteins, and the like. In some embodiments, immunomodulatory cytokines, such as interferons, G-CSF, imiquimod, TNFalpha, and the like, as well as modulators thereof (e.g., blocking antibodies or more potent or longer lasting forms) are used. In some embodiments, immunomodulatory interleukins, such as IL-2, IL-6, IL-7, IL-12, IL-17, IL-23, and the like, as well as modulators thereof (e.g., blocking antibodies or more potent or longer lasting forms) are used. In some embodiments, immunomodulatory chemokines, such as CCL3, CCL26, and CXCL7, and the like, as well as modulators thereof (e.g., blocking antibodies or more potent or longer lasting forms) are used. In some embodiments, immunomodulatory molecules targeting immunosuppression, such as STAT3 signaling modulators, NFkappaB signaling modulators, and immune checkpoint modulators, are used.

In still other embodiments, immunomodulatory drugs, such as immunocytostatic drugs, glucocorticoids, cytostatics, immunophilins and modulators thereof (e.g., rapamycin, a calcineurin inhibitor, tacrolimus, ciclosporin (cyclosporin), pimecrolimus, abetimus, gusperimus, ridaforolimus, everolimus, temsirolimus, zotarolimus, etc.), hydrocortisone (cortisol), cortisone acetate, prednisone, prednisolone, methylprednisolone, dexamethasone, betamethasone, triamcinolone, beclometasone, fludrocortisone acetate, deoxycorticosterone acetate (doca) aldosterone, a non-glucocorticoid steroid, a pyrimidine synthesis inhibitor, leflunomide, teriflunomide, a folic acid analog, methotrexate, anti-thymocyte globulin, anti-lymphocyte globulin, thalidomide, lenalidomide, pentoxifylline, bupropion, curcumin, catechin, an opioid, an IMPDH inhibitor, mycophenolic acid, myriocin, fingolimod, an NF-xB inhibitor, raloxifene, drotrecogin alfa, denosumab, an NF-xB signaling cascade inhibitor, disulfiram, olmesartan, dithiocarbamate, a proteasome inhibitor, bortezomib, MG132, Prol, NPI-0052, curcumin, genistein, resveratrol, parthenolide, thalidomide, lenalidomide, flavopiridol, non-steroidal anti-inflammatory drugs (NSAIDs), arsenic trioxide, dehydroxymethylepoxyquinomycin (DHMEQ), I3C (indole-3-carbinol)/DIM (di-indolmethane) (13C/DIM), Bay 11-7082, luteolin, cell permeable peptide SN-50, IKBa.-super repressor overexpression, NFKB decoy oligodeoxynucleotide (ODN), or a derivative or analog of any thereo, are used. In yet other embodiments, immunomodulatory antibodies or protein are used. For example, antibodies that bind to CD40, Toll-like receptor (TLR), OX40, GITR, CD27, or to 4-1BB, T-cell bispecific antibodies, an anti-IL-2 receptor antibody, an anti-CD3 antibody, OKT3 (muromonab), otelixizumab, teplizumab, visilizumab, an anti-CD4 antibody, clenoliximab, keliximab, zanolimumab, an anti-CD11 a antibody, efalizumab, an anti-CD18 antibody, erlizumab, rovelizumab, an anti-CD20 antibody, afutuzumab, ocrelizumab, ofatumumab, pascolizumab, rituximab, an anti-CD23 antibody, lumiliximab, an anti-CD40 antibody, teneliximab, toralizumab, an anti-CD40L antibody, ruplizumab, an anti-CD62L antibody, aselizumab, an anti-CD80 antibody, galiximab, an anti-CD147 antibody, gavilimomab, a B-Lymphocyte stimulator (BLyS) inhibiting antibody, belimumab, an CTLA4-Ig fusion protein, abatacept, belatacept, an anti-CTLA4 antibody, ipilimumab, tremelimumab, an anti-eotaxin 1 antibody, bertilimumab, an anti-α4-integrin antibody, natalizumab, an anti-IL-6R antibody, tocilizumab, an anti-LFA-1 antibody, odulimomab, an anti-CD25 antibody, basiliximab, daclizumab, inolimomab, an anti-CD5 antibody, zolimomab, an anti-CD2 antibody, siplizumab, nerelimomab, faralimomab, atlizumab, atorolimumab, cedelizumab, dorlimomab aritox, dorlixizumab, fontolizumab, gantenerumab, gomiliximab, lebrilizumab, maslimomab, morolimumab, pexelizumab, reslizumab, rovelizumab, talizumab, telimomab aritox, vapaliximab, vepalimomab, aflibercept, alefacept, rilonacept, an IL-1 receptor antagonist, anakinra, an anti-IL-5 antibody, mepolizumab, an IgE inhibitor, omalizumab, talizumab, an IL12 inhibitor, an IL23 inhibitor, ustekinumab, and the like.

Nutritional supplements that enhance immune responses, such as vitamin A, vitamin E, vitamin C, and the like, are well-known in the art (see, for example, U.S. Pat. Nos. 4,981,844 and 5,230,902 and PCT Publ. No. WO 2004/004483) can be used in the methods described herein.

Similarly, various agents or a combination thereof can be used to treat a cancer. For example, chemotherapy, radiation, epigenetic modifiers (e.g., histone deacetylase (HDAC) modifiers, methylation modifiers, phosphorylation modifiers, and the like), targeted therapy, and the like are well-known in the art.

In some embodiments, chemotherapy is used. Chemotherapy includes the administration of a chemotherapeutic agent. Such a chemotherapeutic agent may be, but is not limited to, those selected from among the following groups of compounds: platinum compounds, cytotoxic antibiotics, antimetabolites, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiments, PARP (e.g., PARP-1 and/or PARP-2) inhibitors are used and such inhibitors are well-known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6 (5H)-phenanthridinone (Trevigen); benzamide (U.S. Pat. Re. 36,397); and NU1025 (Bowman et al.). The mechanism of action is generally related to the ability of PARP inhibitors to bind PARP and decrease its activity. PARP catalyzes the conversion of beta.-nicotinamide adenine dinucleotide (NAD+) into nicotinamide and poly-ADP-ribose (PAR). Both poly(ADP-ribose) and PARP have been linked to regulation of transcription, cell proliferation, genomic stability, and carcinogenesis (Bouchard V. J. et. al. Experimental Hematology, Volume 31, Number 6, June 2003, pp. 446-454 (9); Herceg Z.; Wang Z.-Q. Mutation Research/Fundamental and Molecular Mechanisms of Mutagenesis, Volume 477, Number 1, 2 Jun. 2001, pp. 97-110 (14)). Poly(ADP-ribose) polymerase 1 (PARP1) is a key molecule in the repair of DNA single-strand breaks (SSBs) (de Murcia J. et al. 1997. Proc Natl Acad Sci USA 94:7303-7307; Schreiber V, Dantzer F, Ame J C, de Murcia G (2006) Nat Rev Mol Cell Biol 7:517-528; Wang Z Q, et al. (1997) Genes Dev 11:2347-2358). Knockout of SSB repair by inhibition of PARP1 function induces DNA double-strand breaks (DSBs) that can trigger synthetic lethality in cancer cells with defective homology-directed DSB repair (Bryant H E, et al. (2005) Nature 434:913-917; Farmer H, et al. (2005) Nature 434:917-921). The foregoing examples of chemotherapeutic agents are illustrative, and are not intended to be limiting.

In other embodiments, radiation therapy is used. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In other embodiments, hormone therapy is used. Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In other embodiments, photodynamic therapy (also called PDT, photoradiation therapy, phototherapy, or photochemotherapy) is used for the treatment of some types of cancer. It is based on the discovery that certain chemicals known as photosensitizing agents can kill one-celled organisms when the organisms are exposed to a particular type of light.

In yet other embodiments, laser therapy is used to harness high-intensity light to destroy cancer cells. This technique is often used to relieve symptoms of cancer such as bleeding or obstruction, especially when the cancer cannot be cured by other treatments. It may also be used to treat cancer by shrinking or destroying tumors.

### Inflammatory Disorders

The engineered antigen-binding proteins and/or pharmaceutical compositions described herein can be used, for example, for preventing or treating (reducing, partially or completely, the adverse effects of) an inflammatory disease, such as chronic inflammatory bowel disease, systemic lupus erythematosus, psoriasis, muckle-wells syndrome, rheumatoid arthritis, multiple sclerosis, or Hashimoto's disease, an allergic disease, asthma; an infectious disease; an inflammatory disease such as a TNF-mediated inflammatory disease (e.g., an inflammatory disease of the gastrointestinal tract, such as pouchitis, a cardiovascular inflammatory condition, such as atherosclerosis, or an inflammatory lung disease. The engineered antigen-binding proteins and/or pharmaceutical compositions can be used for suppressing rejection in organ transplantation or other situations in which tissue rejection might occur; for improving immune functions; or for suppressing the proliferation or function of immune cells.

In certain embodiments, the inflammatory disorders include inflammation of any tissue and organs of the body, including musculoskeletal inflammation, vascular inflammation, neural inflammation, digestive system inflammation, ocular inflammation, inflammation of the reproductive system, and other inflammation.

In some embodiments, the musculoskeletal inflammation include conditions affecting skeletal joints, including joints of the hand, wrist, elbow, shoulder, jaw, spine, neck, hip, knew, ankle, and foot, and conditions affecting tissues connecting muscles to bones such as tendons. Examples of such immune disorders, which may be treated with the methods and compositions described herein include, but are not limited to, arthritis (including, for example, osteoarthritis, rheumatoid arthritis, psoriatic arthritis, ankylosing spondylitis, acute and chronic infectious arthritis, arthritis associated with gout and pseudogout, and juvenile idiopathic arthritis), tendonitis, synovitis, tenosynovitis, bursitis, fibrositis (fibromyalgia), epicondylitis, myositis, and osteitis (including, for example, Paget's disease, osteitis pubis, and osteitis fibrosa cystic).

In some embodiments, the ocular immune disorders refers to an immune disorder that affects any structure of the eye, including the eye lids. Examples of ocular immune disorders which may be treated with the methods and compositions described herein include, but are not limited to, blepharitis, blepharochalasis, conjunctivitis, dacryoadenitis, keratitis, keratoconjunctivitis sicca (dry eye), scleritis, trichiasis, and uveitis

In some embodiments, the nervous system immune disorders which may be treated with the methods and compositions described herein include, but are not limited to, encephalitis, Guillain-Barre syndrome, meningitis, neuromyotonia, narcolepsy, multiple sclerosis, myelitis and schizophrenia. Examples of inflammation of the vasculature or lymphatic system which may be treated with the methods and compositions described herein include, but are not limited to, arthrosclerosis, arthritis, phlebitis, vasculitis, and lymphangitis.

In some embodiments, the digestive system immune disorders which may be treated with the methods and pharmaceutical compositions described herein include cholangitis, cholecystitis, enteritis, enterocolitis, gastritis, gastroenteritis, inflammatory bowel disease, ileitis, and proctitis. Inflammatory bowel diseases include, for example, certain art-recognized forms of a group of related conditions. Several major forms of inflammatory bowel diseases are known, with Crohn's disease (regional bowel disease, e.g., inactive and active forms) and ulcerative colitis (e.g., inactive and active forms) the most common of these disorders. In addition, the inflammatory bowel disease encompasses irritable bowel syndrome, microscopic colitis, lymphocytic-plasmocytic enteritis, coeliac disease, collagenous colitis, lymphocytic colitis and eosinophilic enterocolitis. Other less common forms of IBD include indeterminate colitis, pseudomembranous colitis (necrotizing colitis), ischemic inflammatory bowel disease, Behcet's disease, sarcoidosis, scleroderma, IBD-associated dysplasia, dysplasia associated masses or lesions, and primary sclerosing cholangitis.

In some embodiments, the reproductive system immune disorders which may be treated with the methods and pharmaceutical compositions described herein include, but are not limited to, cervicitis, chorioamnionitis, endometritis, epididymitis, omphalitis, oophoritis, orchitis, salpingitis, tubo-ovarian abscess, urethritis, vaginitis, vulvitis, and vulvodynia.

In some embodiments, the inflammatory disorders include acute disseminated alopecia universalise, Behcet's disease, Chagas' disease, chronic fatigue syndrome, dysautonomia, encephalomyelitis, ankylosing spondylitis, aplastic anemia, hidradenitis suppurativa, autoimmune hepatitis, autoimmune oophoritis, celiac disease, Crohn's disease, diabetes mellitus type 1, type 2 diabetes, giant cell arteritis, Goodpasture's syndrome, Graves' disease, Guillain-Barre syndrome, Hashimoto's disease, Henoch-Schonlein purpura, Kawasaki's disease, lupus erythematosus, microscopic colitis, microscopic polyarteritis, mixed connective tissue disease, Muckle-Wells syndrome, multiple sclerosis, myasthenia gravis, opsoclonus myoclonus syndrome, optic neuritis, ord's thyroiditis, pemphigus, polyarteritis nodosa, polymyalgia, rheumatoid arthritis, Reiter's syndrome, Sjogren's syndrome, temporal arteritis, Wegener's granulomatosis, warm autoimmune haemolytic anemia, interstitial cystitis, Lyme disease, morphea, psoriasis, sarcoidosis, scleroderma, ulcerative colitis, and vitiligo.

The methods and compositions described herein may be used to treat T-cell mediated hypersensitivity diseases having an inflammatory component. Such conditions include contact hypersensitivity, contact dermatitis (including that due to poison ivy), uticaria, skin allergies, respiratory allergies (hay fever, allergic rhinitis, house dust mite allergy) and gluten-sensitive enteropathy (Celiac disease).

Other immune disorders which may be treated with the methods and pharmaceutical compositions include, for example, appendicitis, dermatitis, dermatomyositis, endocarditis, fibrositis, gingivitis, glossitis, hepatitis, hidradenitis suppurativa, iritis, laryngitis, mastitis, myocarditis, nephritis, otitis, pancreatitis, parotitis, pericarditis, peritonitis, pharyngitis, pleuritis, pneumonitis, prostatitis, pyelonephritis, and stomatitis, transplant rejection (involving organs such as kidney, liver, heart, lung, pancreas (e.g., islet cells), bone marrow, cornea, small bowel, skin allografts, skin homografts, and heart valve xenografts, serum sickness, and graft vs host disease), acute pancreatitis, chronic pancreatitis, acute respiratory distress syndrome, Sexary's syndrome, congenital adrenal hyperplasis, nonsuppurative thyroiditis, hypercalcemia associated with cancer, pemphigus, bullous dermatitis herpetiformis, severe erythema multiforme, exfoliative dermatitis, seborrheic dermatitis, seasonal or perennial allergic rhinitis, bronchial asthma, contact dermatitis, atopic dermatitis, drug hypersensitivity reactions, allergic conjunctivitis, keratitis, herpes zoster ophthalmicus, iritis and oiridocyclitis, chorioretinitis, optic neuritis, symptomatic sarcoidosis, fulminating or disseminated pulmonary tuberculosis chemotherapy, idiopathic thrombocytopenia purpura in adults, secondary thrombocytopenia in adults, acquired (autoimmune) haemolytic anemia, regional enteritis, autoimmune vasculitis, multiple sclerosis, chronic obstructive pulmonary disease, solid organ transplant rejection, sepsis. Preferred treatments include treatment of transplant rejection, rheumatoid arthritis, psoriatic arthritis, multiple sclerosis, Type 1 diabetes, asthma, inflammatory bowel disease, systemic lupus erythematosus, psoriasis, chronic obstructive pulmonary disease, and inflammation accompanying infectious conditions (e.g., sepsis).

### Clinical Efficacy/Response to a Therapy

Clinical efficacy can be measured by any method known in the art. For example, the response to a therapy relates to any response of the cancer, e.g., a tumor, to the therapy, preferably to a change in tumor mass and/or volume after initiation of neoadjuvant or adjuvant chemotherapy. Tumor response may be assessed in a neoadjuvant or adjuvant situation where the size of a tumor after systemic intervention can be compared to the initial size and dimensions as measured by CT, PET, mammogram, ultrasound or palpation and the cellularity of a tumor can be estimated histologically and compared to the cellularity of a tumor biopsy taken before initiation of treatment. Response may also be assessed by caliper measurement or pathological examination of the tumor after biopsy or surgical resection. Response may be recorded in a quantitative fashion like percentage change in tumor volume or cellularity or using a semi-quantitative scoring system such as residual cancer burden (Symmans et al., J. Clin. Oncol. (2007) 25:4414-4422) or Miller-Payne score (Ogston et al., (2003) Breast (Edinburgh, Scotland) 12:320-327) in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of tumor response may be performed early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed.

In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR-CR+PR+SD over 6 months. In some embodiments, the CBR for a particular anti-immune checkpoint therapeutic regimen is at least 25%, 30%, 35%, 40%, 45%, 50%, 55%, 60%, 65%, 70%, 75%, 80%, 85%, or more.

Additional criteria for evaluating the response to a cancer therapy are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include probability of survival, probability of metastasis within a given time period, and probability of tumor recurrence.

For example, in order to determine appropriate threshold values, a particular anti-cancer therapeutic regimen can be administered to a population of subjects and the outcome can be correlated to biomarker measurements that were determined prior to administration of any cancer therapy. The outcome measurement may be pathologic response to therapy given in the neoadjuvant setting. Alternatively, outcome measures, such as overall survival and disease-free survival can be monitored over a period of time for subjects following the cancer therapy for whom biomarker measurement values are known. In certain embodiments, the same doses of anti-cancer agents are administered to each subject. In related embodiments, the doses administered are standard doses known in the art for anti-cancer agents. The period of time for which subjects are monitored can vary. For example, subjects may be monitored for at least 2, 4, 6, 8, 10, 12, 14, 16, 18, 20, 25, 30, 35, 40, 45, 50, 55, or 60 months. Biomarker measurement threshold values that correlate to outcome of a cancer therapy can be determined using methods such as those described in the Examples section.

### Kits

In some embodiments, the antigen-binding proteins of the present disclosure are provided in a kit. In various aspects, the kit comprises the antigen-binding protein(s) as a unit dose. For purposes herein “unit dose” refers to a discrete amount dispersed in a suitable carrier. In various aspects, the unit dose is the amount sufficient to provide a subject with a desired effect, e.g., inhibition of tumor growth, reduction of tumor size, treatment of cancer. Accordingly, provided herein are kits comprising an antigen-binding protein of the present disclosure optionally provided in unit doses. In various aspects, the kit comprises several unit doses, e.g., a week or month supply of unit doses, optionally, each of which is individually packaged or otherwise separated from other unit doses. In some embodiments, the components of the kit/unit dose are packaged with instructions for administration to a patient. In some embodiments, the kit comprises one or more devices for administration to a patient, e.g., a needle and syringe, and the like. In some aspects, the antigen-binding protein of the present disclosure, a pharmaceutically acceptable salt thereof, a conjugate comprising the antigen-binding protein, or a multimer or dimer comprising the antigen-binding protein, is pre-packaged in a ready to use form, e.g., a syringe, an intravenous bag, etc. In some aspects, the kit further comprises other therapeutic or diagnostic agents or pharmaceutically acceptable carriers (e.g., solvents, buffers, diluents, etc.), including any of those described herein. In particular aspects, the kit comprises an antigen-binding protein of the present disclosure, along with an agent, e.g., a therapeutic agent, used in chemotherapy or radiation therapy.

The following examples are given merely to illustrate the present disclosure and not in any way to limit its scope.

### EXAMPLES

### Example 1: CTLA4 Blockade Assay

CTLA4 blockade assays described herein used the commercially available kit, CTLA4 blockade bioassay (cat. #JA3001 and JA3005; Promega Corporation, Madison, WI). CTLA4, also known as CD152, is an immune inhibitory receptor constitutively expressed on regulatory T cells (Tregs) and upregulated in activated T cells. CTLA4 plays a critical role in regulating immune responses to tumor antigens and autoantigens. CTLA4 is the counterpart of the co-stimulatory B7-CD28 pathway. When CTLA4 expression is upregulated on the surface of T cells, the T cells bind B7 with a higher avidity, and thus out-compete the positive co-stimulatory signal from CD28. In addition, engagement of CTLA4 by either of its ligands, CD80 (B7-1) or CD86 (B7-2) on an adjacent antigen presenting cell (APC) inhibits CD28 co-stimulation of T cell activation, cell proliferation and cytokine production.

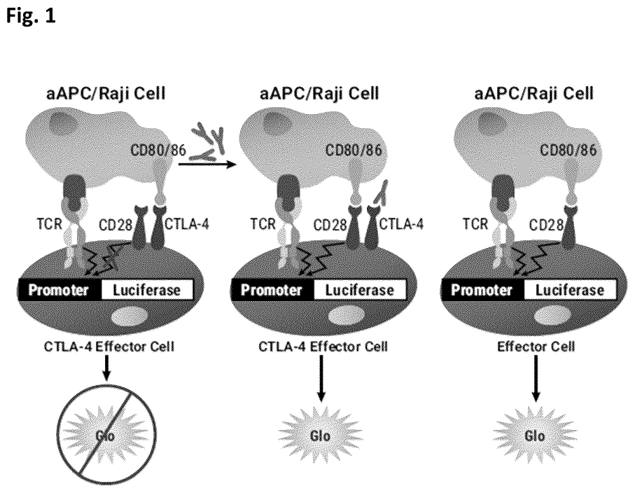

The CTLA4 blockade assay involved two cell lines: Jurkat T cells (immortalized lymphocytic leukemia T cells, also referred to as the CTLA4 effector cells) and Raji cells (also referred to as the antigen-presenting cells (APC)). Jurkat T cells express human CTLA4 and a luciferase reporter driven by a native promoter which responds to TCR/CD28 activation. Raji cells express an engineered cell surface protein designed to activate cognate TCRs in an antigen-independent manner and endogenously expressing CTLA4 ligands CD80 (B7-1) and CD86 (B7-2), collectively called the B7 ligands. When the two cell types are co-cultured, CTLA4 competes with CD28 for their shared ligands, CD80 and CD86, and thus inhibits CD28 pathway activation and promoter-mediated luminescence. Addition of an anti-CTLA4 antibody blocks the interaction of CTLA4 with its ligands CD80 and CD86 and results in promoter-mediated luminescence (FIG. 1).

Jurkat cells can also be activated by anti-CD3, anti-CD28, or anti-CD3 with cell surface B7 ligands to express IL-2. Thus, in addition to the luminescence, the expression level of IL-2 protein is a metric of activation. Accordingly, when Jurkat cells are co-cultured with Raji cells that endogenously express B7 ligands and now engineered to express anti-CD3, the Jurkat cells are activated and express both IL-2 and luciferase. The expression levels of IL-2 and luciferase are linearly correlated with each other and with activation. However, the presence of CTLA4 extracellular domain, which binds the B7 ligands and blocks the Jurkat cell activation, abrogates both IL-2 and luciferase expression. Agents that block CTLA4 function, e.g., blocking the CTLA4's binding of B7 ligands, enable the Jurkat cell expression of IL-2 and luciferase. There is a linear correlation between IL-2 expression, luciferase expression/activity, and blocking efficacy of the anti-CTLA4 agent. The correlation between the CTLA4 blockade as measured by this assay and the primary T cell activation has been well documented, further validating the applicability of this assay (Waight et al. (2018) Cancer Cell 33:1033-1047).

While the activity curves in both assays are expected to have a similar profile, there is an effect from Fc receptors in T cell activation (Waight et al. (2018) Cancer Cell 33:1033-1047). Specifically, the anti-CTLA4 antibodies in certain in vitro T cell activation settings with antigen presenting cells show a biological effect dependent on Fc receptor interactions. Raji cells express FcγRII (CD32), and blockade of anti-CTLA4 antibody Fc interactions with FcγRII through either an anti-CD32 antibody or through deletion of Fc domains leads to an attenuation of an anti-CTLA4 antibody's activity as well as a decrease in the absolute level of response. This attenuation of an anti-CTLA4 antibody's activity has been reported to extend to antigen stimulation of primary T cell activation, where the FcγRIIIA (CD16) receptor was essential and not FcγRI, FcγRIIA/B, or FcγRIIB (Waight et al. (2018) Cancer Cell 33:1033-1047). Raji cells used in the Promega assay do not express FcγRIIIA (CD16) and no negative activity dampening effect is seen with the anti-CD16 antibodies although the anti-CD32 antibodies are strong attenuators of anti-CTLA biological action. Thus, it is widely accepted that the interaction between the Fc region of a CTLA4 antibody with the Fc receptors is important for its CTLA4-blocking activity (Bulliard et al. (2013) J of Exp Medicine 9:1685-1693; Waight et al. (2018) Cancer Cell 33:1033-1047; Ingram et al. (2018) Proc Natl Acad Sci USA 115:3912-3917; Vargas et al. (2018) Cancer Cell 33:1-15).

The CTLA4 blockade assay described herein involved 120 samples on 2 plates of the 96-well plate. The assay was performed with samples in duplicate unless otherwise noted, in a titration curve of the protein concentration or the concentration of binding valency (number of antigen-binding sites). The standard control anti-CTLA4 antibodies were also assayed in duplicate in titration and within the test sample assay plate. For exceptional situations, the control anti-CTLA4 antibody titration curves from another plate, but within in the same assay kit, were used. The Promega CTLA4 Blockade Bioassay is designed and fitted for a Relative Luciferase Activity (RLU) readout. The activities of samples were reported herein as the ratio of Test Agent Relative Luciferase Units/No Test Agent Relative Luciferase Units (background luciferase units representative of basal levels of T cell activation). The assays were incubated from 12 to 16 hours. While variation in Relative Luciferase Units occurred among experiments, but the ratios of the CTLA4-blocking activity remained constant. In some experiments, rather than measuring luciferase activity, the assay well media was assayed for human IL-2 levels. Conventional Human IL-2 ELISA assays were used. Generally, the entire contents of the assay well were analyzed directly for IL-2.

The control anti-Human CTLA4 antibodies used were: Cat. No. JA1020 from Promega, ipilimumab from SelleckChem, and L3D10 from BioLegends.

### Example 2: Importance of Fc Receptor in CTLA4 Blocking Activity of the Anti-CTLA4 Antibodies

In order to develop novel CTLA4-binding proteins with increased potency and safety profiles, it was hypothesized that there are sites spanning the extracellular domain of CTLA4 that are amenable to binding proteins which can allosterically block CTLA4 ligand binding and that affect the biological signaling of the CTLA4 receptor. It was also hypothesized that such signaling can be the basis of novel potent immune onco-therapeutics.

Experimental antibodies and their derivatives gene sequences, sequence confirmation, expression, purification and analytical protein analysis were produced by either Proteos (Kalamazoo, MI) or Absolute Antibody™ (Oxford, UK). The antibodies' and derivatives' gene sequences were synthesized, inserted into proprietary expression vectors, expressed in 293 cells by standard transient transfection protocols and were purified by either protein A columns, or Ni columns when a His6 tag (SEQ ID NO: 78) was used. All experimental antibodies had less than 0.1 EU per mg.

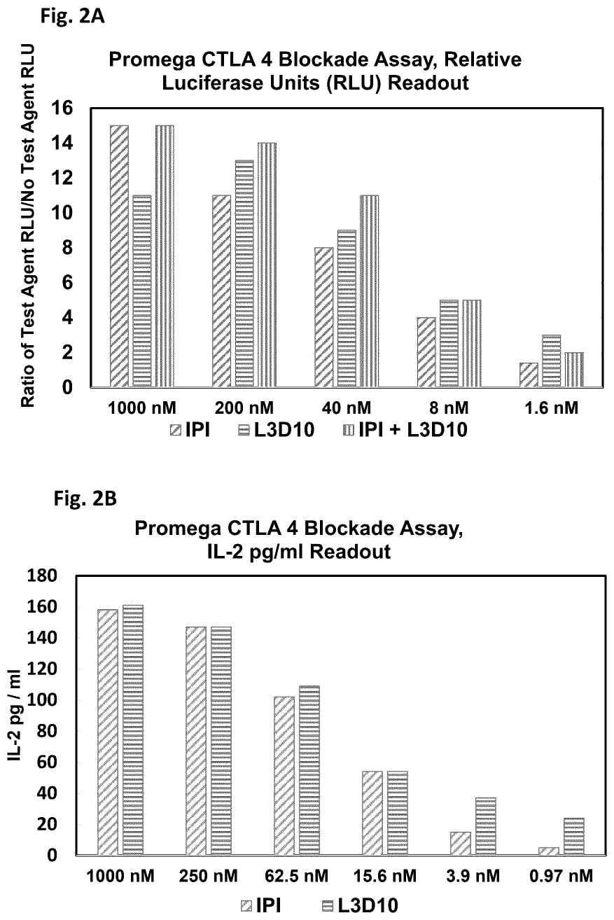

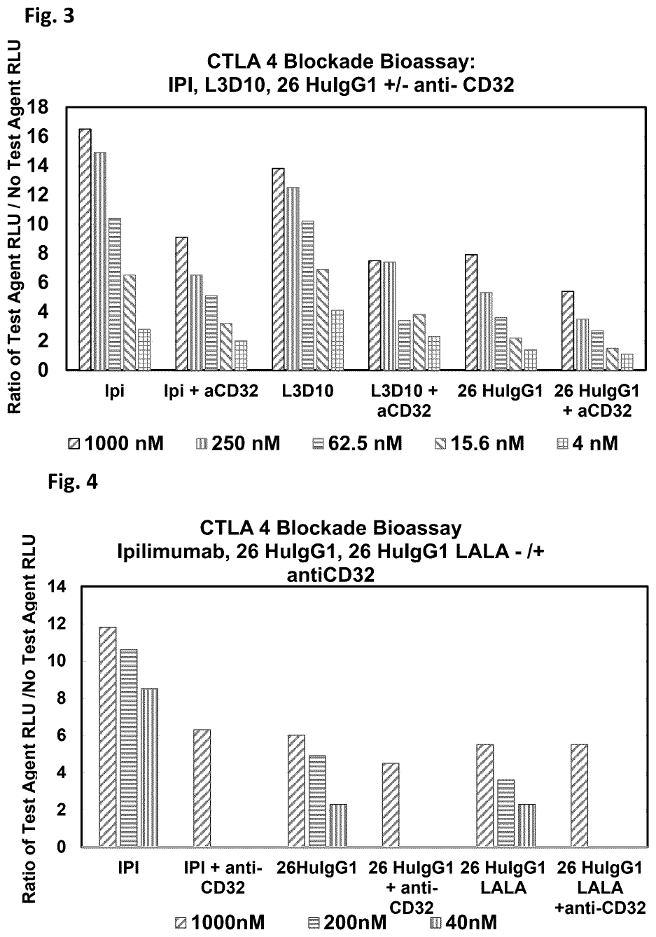

FIG. 2A and FIG. 2B show the Promega CTLA4 Blockade Bioassay of titration curve dose response of ipilimumab (IPI) (Selleckchem, Cat. No. A2001) and L3D10 (BioLegends) executed on 2 separate assays where one assay set was readout in the standard luciferase activity assay (FIG. 2A) and the other was readout as levels of IL-2 protein expressed (FIG. 2B). The RLU ratios (10⁻¹⁵) were consistent with those reported in the Promega technical manual and consistent with a similar activity titration curve for Promega's anti-CTLA4 antibody, JA1020 (data not shown). RLU ratios of 30+over background have been observed when Jurkat effector cells alone are incubated with anti-CD3, anti-CD28 and an anti-Fc crosslinking antibody. The titration profiles of ipilimumab and L3D10 each showed similar half max activity concentrations of approximately 50 nM for either luciferase or IL-2 readouts.

Ipilimumab and L3D10 (May et al. (2005), Du et al. (2018)) both bind close to the ligand binding site of CTLA4 for B7s, ¹³⁴MYPPPY¹³⁹ (SEQ ID NO: 1), however both ipilimumab and L3D10 have different biochemical and biological properties (Du et al. (2018a), Du et al. (2018b)) that likely arise from their respective unique sequence structure. As shown in FIG. 2B, the blockade activities of both ipilimumab and L3D10 are likely mechanistically similar as their activities are not additive when the antibody titration series are added together. Concentrations of ipilimumab as high as 5000 nM and also concentrations of ipilimumab and L3D10 admixture of 2000 nM each have been tested and their titration curves appear to plateau in the range of 1000 nM and the activity ratios stayed in the 10⁻¹⁵ range.

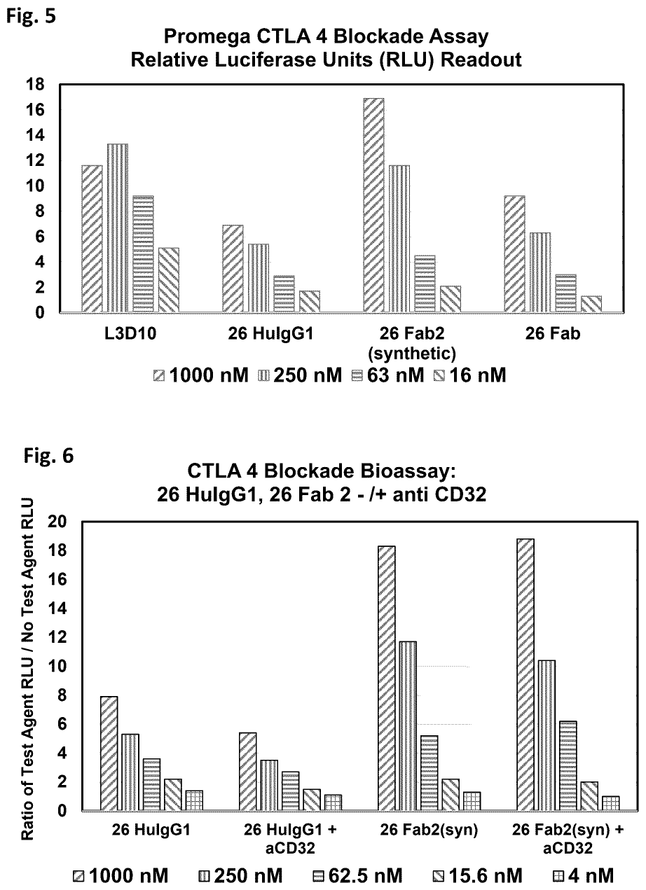

It is identified herein that the binding sites along CTLA4 distal to the CTLA4's ligand binding sites have novel properties. One such site is ⁶⁵SICT⁶⁸ (SEQ ID NO: 2). An antibody (hereinafter “antibody 26” or “26 antibody”), an anti-human CTLA4 mouse antibody described in patent U.S. Pat. No. 7,034,121B2 (which is incorporated herein by reference) binds this site and allosterically blocks CTLA4 binding to B7s. A chimeric antibody was generated by fusing the VH and VL domains of the antibody 26 to a human IgG1 scaffold, essentially the same IgG1 framework of ipilimumab. This chimeric antibody is referred to as 26 HuIgG1. As shown in FIG. 3, 26 HuIgG1 was active in the CTLA4 blockade assay, however the activity maximum observed was only half that of ipilimumab or L3D10. Additionally, the half maximal activity for 26 HuIgG1 was about 100 nM compared to approximately 50 nM for ipilimumab. When the anti-CD32 antibody was present at 10 μg per ml and blocked FcγRII binding, the entire activity titration curve for ipilimumab, L3D10, and 26 HuIgG1 was displaced about 50% to a lower RLU ratio (FIG. 3). The action of anti-CD32 antibodies demonstrated that the Fc region of the anti-CTLA4 antibodies is essential to activity although it may not be as simple as Fc binding to receptor.

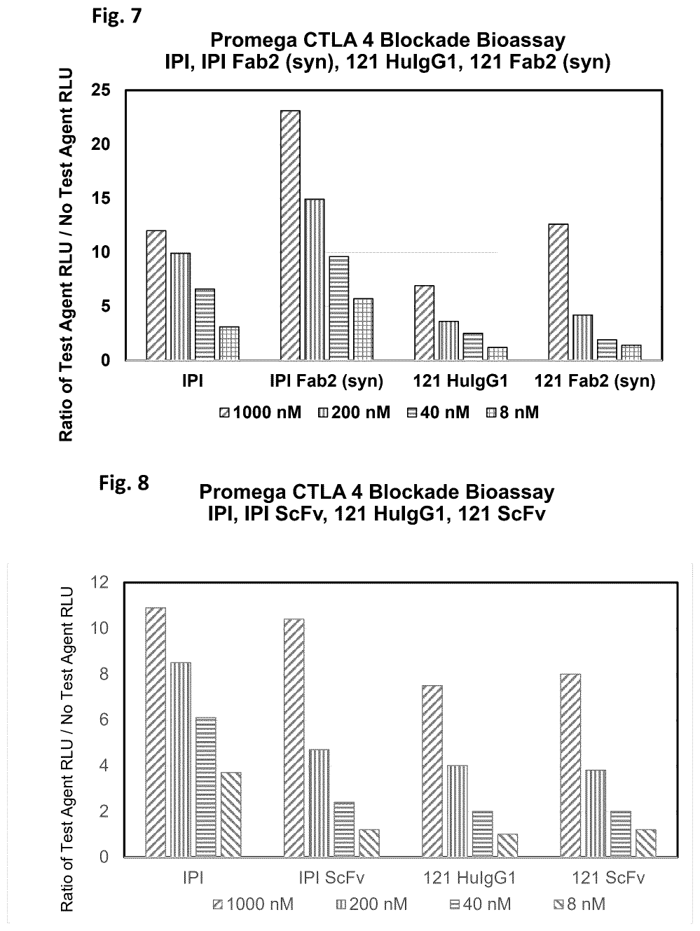

FIG. 4 shows a comparison of the activities of 26 HuIgG1 with 26 HuIgG1 with a LALA mutation, which abolishes the FcγR and C1q interaction (Schlothauer et al. (2016) Protein Engineering, Design & Selection 10:457-466). Within the experimental resolution of this assay, there is not substantial effect for eliminating Fc receptor binding by this mutation nor does adding an anti-CD32 antibody have any detectable effect. Notably for ipilimumab, the addition of anti-CD32 reduced its activity by almost 50%, consistent with the results observed in FIG. 3, and by Promega and Waight et al. (2018) Cancer Cell 33:1033-1047. Thus, the interaction between FcγR and the anti-CTLA4-antibody, which occurs via the Fc region of the antibody, is required for full CTLA4-blocking activity.

### Example 3: Surprising and Unexpected Activity of the CTLA4-Binding Proteins that Lack the Fc Region

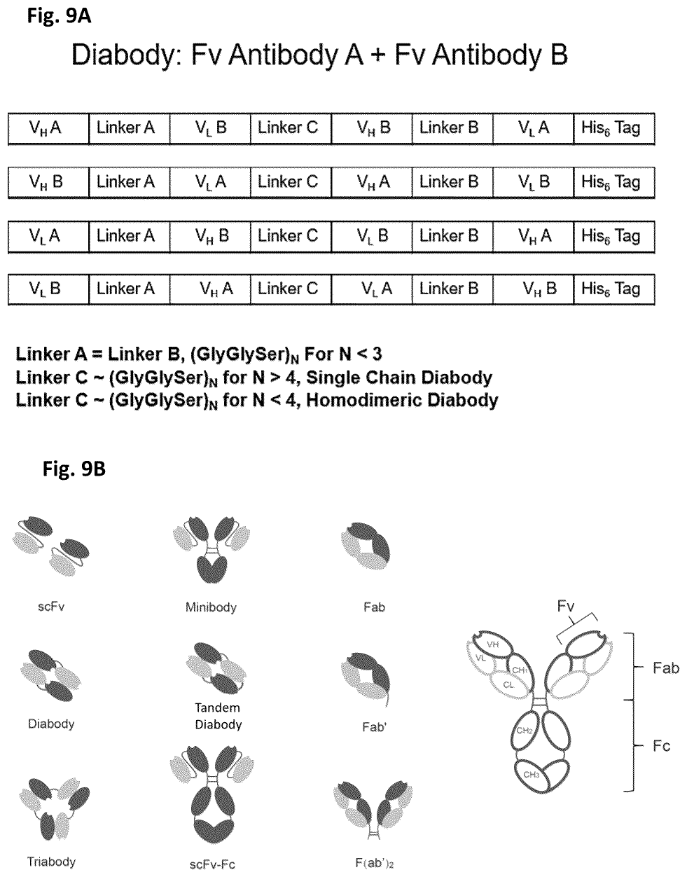

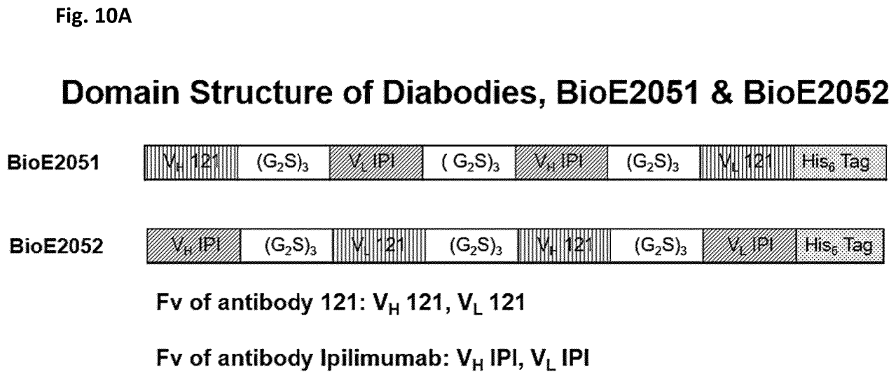

26 antibody derivatives with no C_{H}2 C_{H}3 domains and thereby lacking any means to bind to FcγR. 26 F(ab′)2 (synthetic) were engineered by using a fungal protein dimerization domain (a proprietary technology of Absolute Antibody™) to synthetically construct a dimer of two 26 Fabs, where each 26 Fab′ is a molecularly engineered 26 V_{H} C_{H}1 and 26 V_{L}C_{L}. The experiment of engineered antibody derivatives of 26 HuIgG1 without the Fc region, specifically 26 F(ab′)2 (syn) and 26 Fab′, unexpectedly showed the activity curves that were more active (FIG. 5) than either 26 HuIgG1 or 26 HuIgG1+anti-CD32. Strikingly, FIG. 5 showed that the F(ab′)2 form of 26 has its activity titration curve shifted to a higher level of activity, more than 2× of 26 HuIgG1 and about 10+% more active than L3D10. However, the displacement of 26 F(ab′)2 to a higher activity was not uniform through the titration but is evident at high concentrations. The addition of the anti-CD32 antibody at 10 μg per ml to the titrations of 26 F(ab′)2 (syn) had no effect on the activity titration profile (FIG. 6). 26 Fab′ had an activity titration curve similar to 26 HuIgG1. Also notable is that 26 Fab′ is a monovalent binder while 26HuIgG1 is a bivalent and the activity titration curve of 26 Fab′ is upshifted compared to 26 HuIgG1.

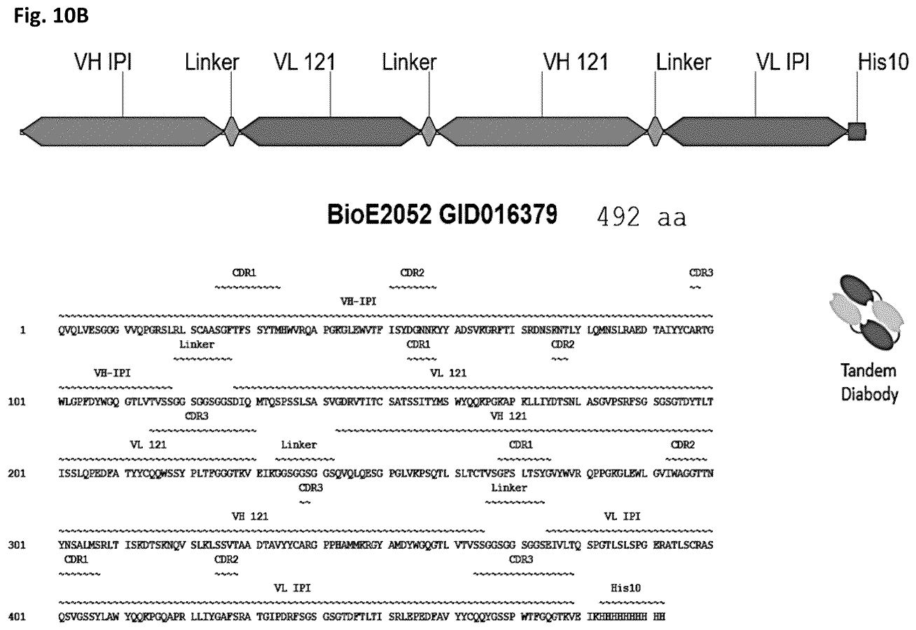

The structural effect observed for 26 Fv and Fc domains was not case specific only for the derivatives of the 26 antibody. As demonstrated herein, the ipilimumab activity in the CTLA4 blockade assay was decreased by anti-CD32 antibodies. Thus, binding to FcγRII is essential for the full ipilimumab activity. Nonetheless, the Ipi F(ab′)2 and Ipi ScFv constructs showed that eliminating the Fc region was not equivalent to blocking its interaction with FcγRII via anti-CD32 antibody. FIG. 7 shows the activity titration curves of Ipi F(ab′)2 (constructed as a synthetic dimer of Ipi Fabs with Absolute Antibody™) and a conventional scFv of the Ipi Fv regions in Ipi V_{L} (Gly₄Ser)₃ Ipi V_{H} (“(Gly4Ser)3” disclosed as SEQ ID NO: 98). As shown in FIG. 7, the activity titration curve for Ipi F(ab′)2 (syn) was twice the magnitude of ipilimumab for the high concentrations of the curve, but less so, albeit still more active at the lower concentrations. At the highest concentration of 1000 nM, the activity of Ipi ScFv was equivalent to ipilimumab but less at the lower concentrations. The activities of Ipi ScFv at the 200, 40, and 8 nM concentrations were about 50% of that seen for the ipilimumab control in this assay, similar to that observed for the combination of ipilimumab+anti-CD32 antibody.

The addition of an Fc region to an anti-CTLA4 binding domain may “bridge” through Fc receptor binding to accessory immune cells (Waight et al. (2018) Cancer Cell 33:1033-1047), in this assay Raji cells, which can affect the resulting biology (T cell activation) of blocking CTLA4 ligand binding. Waight et al. (2018) have shown that this bridging is a structural process, since mutations that degrade or enhance Fc binding to the receptor consequently affect the activity titration curve seen for an anti-CTLA4 antibody; reducing Fc binding affinity downshifts the entire titration curve. However, in therapeutic applications of anti-CTLA4 biologics, the addition of an Fc region could be adding positive or negative modulation of T cell activation and other inadvertent activities such as Fc-directed depletion of CTLA4 expressing cells, e.g., TREG cells. These biological activities are additional to the basic action of blocking CTLA4 from binding its ligand, but a more functionally focused anti-CTLA4. As shown in FIG. 7, Ipi F(ab′)2 (syn) can be almost twice as active as ipilimumab in this CTLA4 blockade assay. Note that the activity titration curves start at concentrations as high as 5000 nM, and ipilimumab plateaus at 1000 nM; the addition of an Fc region affects and limits a conventional anti-CTLA4 antibody's blockade potency.

For the F(ab′)2, Fab′, and ScFv forms of 26 antibody and ipilimumab, there appears to be a concentration dependent effect on activity, evident on close examination of their activity titration curves. In comparing the activity curves of ipilimumab and 26 HuIgG1 with their sub-antibody derivatives F(ab′)2, Fab′, and ScFv forms, there is a subtle inflection, a decrease in activity generally below 200 nM.

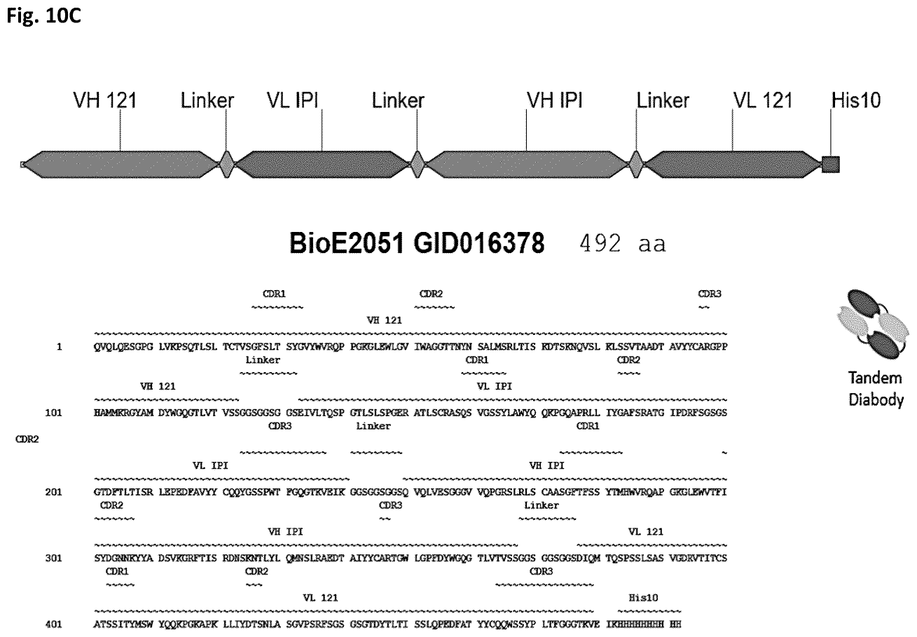

121 Hu IgG1 is the human form of mouse antibody 26. Table 3 shows the human Vₖ light chain for the humanized form of antibody 26. Table 3 also shows the human V_{H} heavy chain of the humanized antibody 26. The 121 HuIgG1 is the assembly of these into a full antibody. Exclusive of the V_{L} and V_{H} sequences are the identical sequences for IgG1 (C_{L} C_{H}1, C_{H}2, C_{H}3) found in ipilimumab. Thus, ipilimumab and 121 HuIgG1 differ only in the V_{L} and V_{H} variable domains. FIG. 7 and FIG. 8 show the activity titration curve for 121 HuIgG1, which is displaced to a lower activity curve compared to ipilimumab and consistent for it being a humanized form of 26 HuIgG1.

121 ScFv is a single chain form containing 121 VL and 121 VH: 121VL-(Gly₄Ser)₃-121VH-His₆ (“(Gly4Ser) 3” disclosed as SEQ ID NO: 98).

FIG. 7 and FIG. 8 show the activity titration curve for 121 ScFv and 121 HuIgG1.

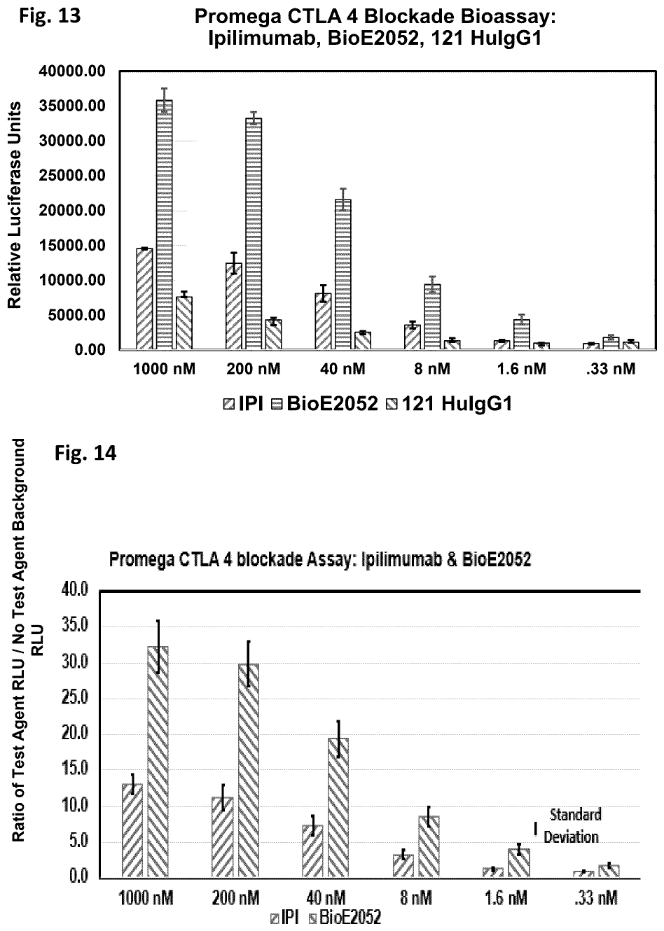

FIG. 16 shows the ratios of RLU for ipilimumab, Ipi ScFv, and Ipi F(ab′)2. Ipi ScFv showed the lower activity, but F(ab′)2 showed higher activity. As described above, this was contrary to the lowered CTLA4-blocking activity observed when the anti-CD32 antibody was added to ipilimumab to block the FcγRII receptor from interacting with the ipilimumab Fc region. As also described above, this was contrary to the published work by Waight et al. (2018) and others listed above as well as Promega. Thus, this increased activity for F(ab′)2 was surprising and unexpected.

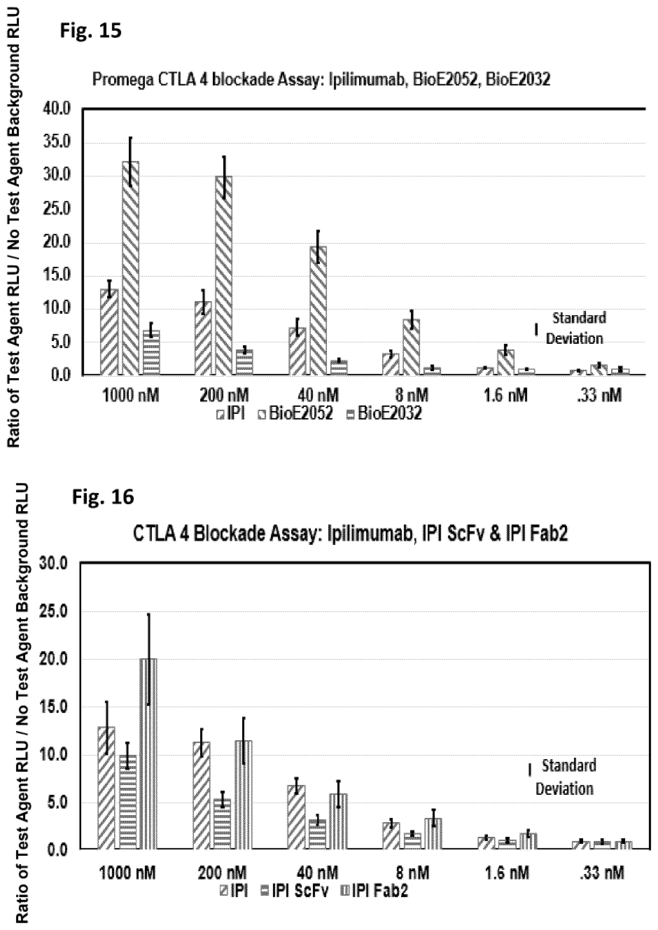

FIG. 17 shows the CTLA4-blocking activity of BioE2021 (Ipi scFv), BioE2031 (121 scFv), BioE2022 (Ipi F(ab′)2), BioE2033 (121 F(ab′)2), and ipilimumab. The experiment was done in triplicate, except the ipilimumab control which is only assayed in duplicate. As described above, the assay is formatted such that only 60 samples can be assay per plate and therefore limits the number of samples formally assayed under similar experimental conditions. Each pre-packed Promega assay allows for 120 samples, 2 plates of 60. BioE2021=Ipi ScFv, BioE2031=121 ScFv, BioE2022=Ipi F(ab′)2, BioE2033=121 F(ab′)2.

### Example 4: Biparatopic CTLA4-Binding Proteins that Modulate CTLA4 and Activate T Cells

The observations described herein suggested that engineering better avidity, thereby increasing the local molecular concentration of an anti-CTLA4 binding protein, could lead to much greater potency. In addition, with the antibody designs that do not include the Fc region, a more pristine CTLA4 therapeutic may be possible without the consequences of Fc-driven immune accessory cell activity, notably TREG depletion. With this aim, biparatopic anti-CTLA4 constructs that could bind 2 different epitopes on CTLA4 were designed.

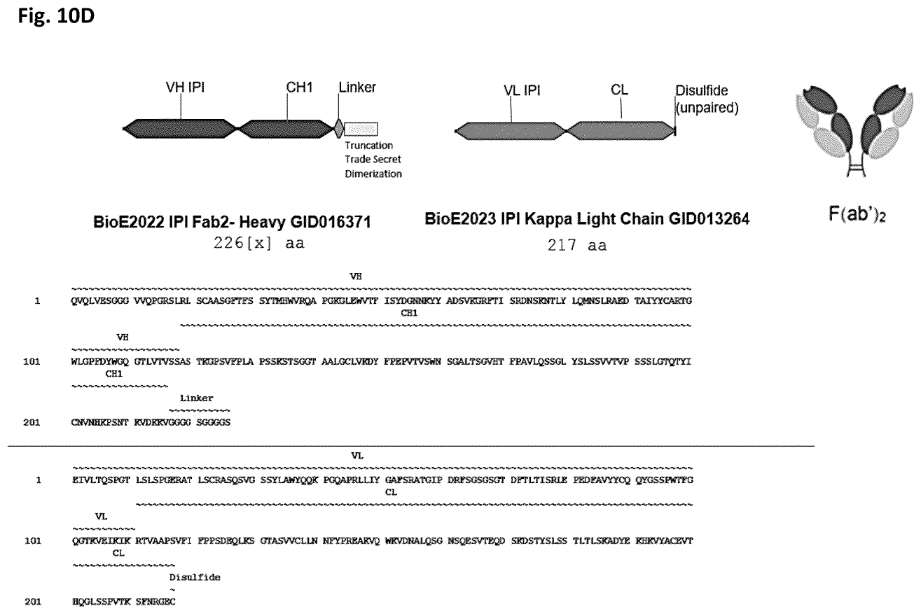

FIG. 9A shows a schematic diagram of the exemplary diabody designs (see Kipriyanov et al. (1999), Volkel et al. (2001), Gall et al. (2004), and Reusch et al. (2014). The basic diabody comprises just the VL and VH domains arrange to fold either upon itself, monomeric form, or dimerize with itself, homodimeric diabody. The linkers between the VL and VH domains in conjunction with the amino acid sequence unique to each VL VH domains determine whether the diabody is monomeric or dimeric. FIG. 9A shows 4 forms with exemplary linker variations. These forms are a mere guide for construction and the optimal constructs cannot be predicted.

FIG. 10A shows a schematic diagram of the exemplary 2 constructs implemented; BioE2051 and BioE2052. BioE2051 and BioE2052 gene constructs were synthesized by conventional means, inserted in proprietary mammalian expression vectors of Absolute Antibody™, expressed in 293 cells. Protein product was purified by Ni column and analyzed by size exclusion chromatography (SEC). BioE2051 production yield was low, approximately 0.5 mg per 250 liter 293 cells, and revealed 2 peaks on SEC, labelled as BioE2051 P1, BioE2051 P2. BioE2052's final yield was 4 mg per 250 ml 293 cells and revealed a single peak on SEC. BioE2051 P1, BioE2051 P2 and BioE2052 had endotoxin levels of <0.1 Endotoxin Unit per mg.

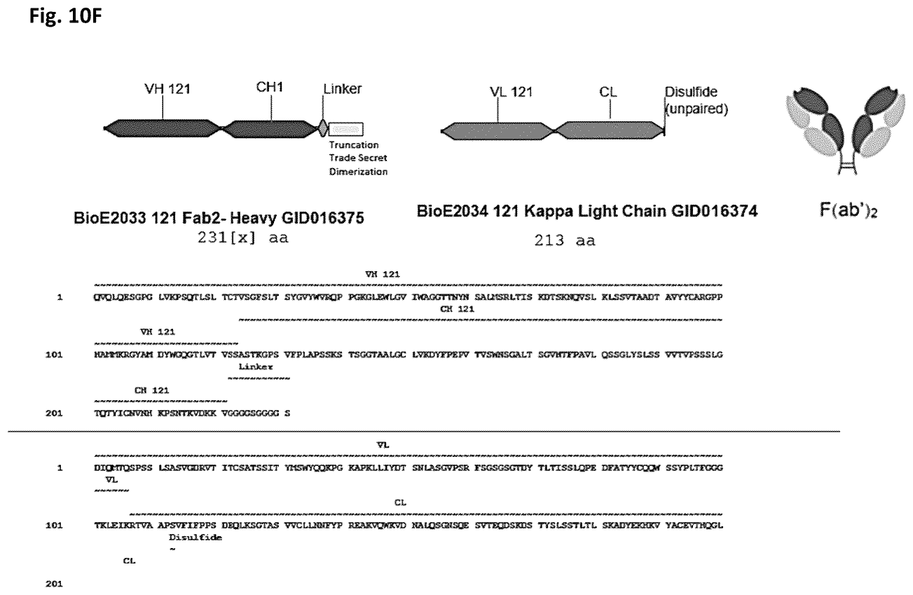

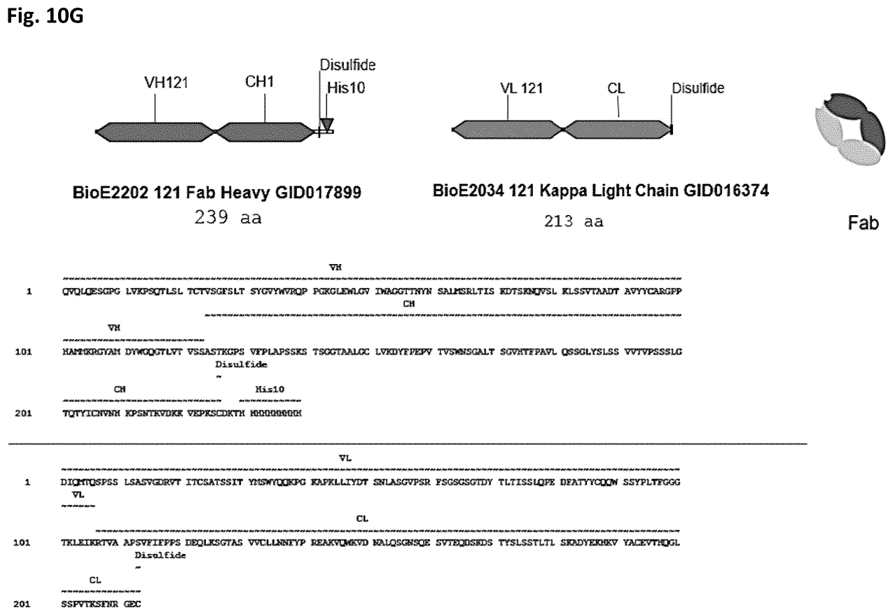

BioE2051 P1, BioE2052 P2, and BioE2052 were assayed in the Promega CTLA4 blockade assay and all three diabody constructs were significantly more active throughout an activity titration curve than either ipilimumab or 121 HuIgG1 (FIG. 11). BioE2052 was further analyzed and compared to a mixture of Ipi ScFv and 121ScFv (FIG. 12). Ipi ScFv and 121 ScFv activity curves were additive but considerably less active than their covalent combination in a diabody form. BioE2052's activity is plateaued as, the lower concentration portion of the curve, after the first dilution point, showed activities that are 3 plus times greater than the corresponding concentration mixture of Ipi ScFv and 121 ScFv.

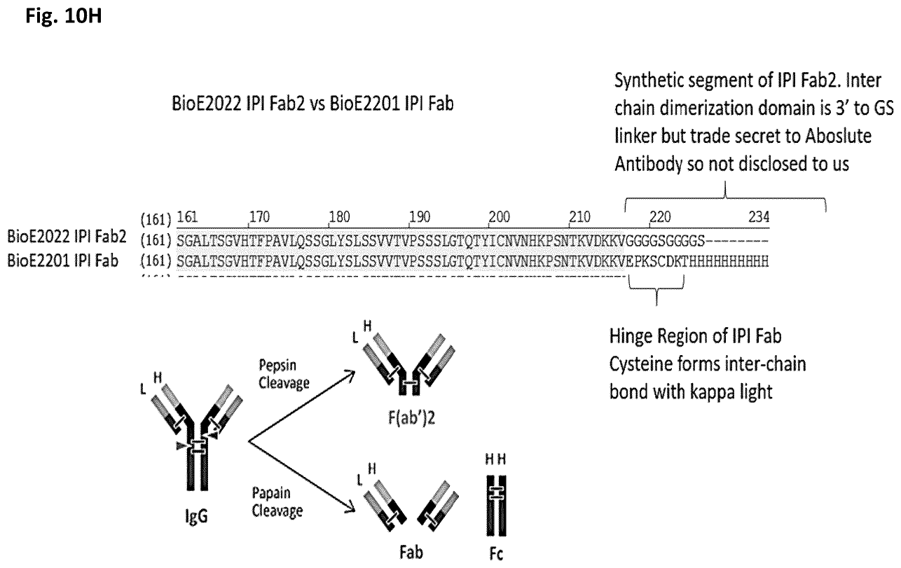

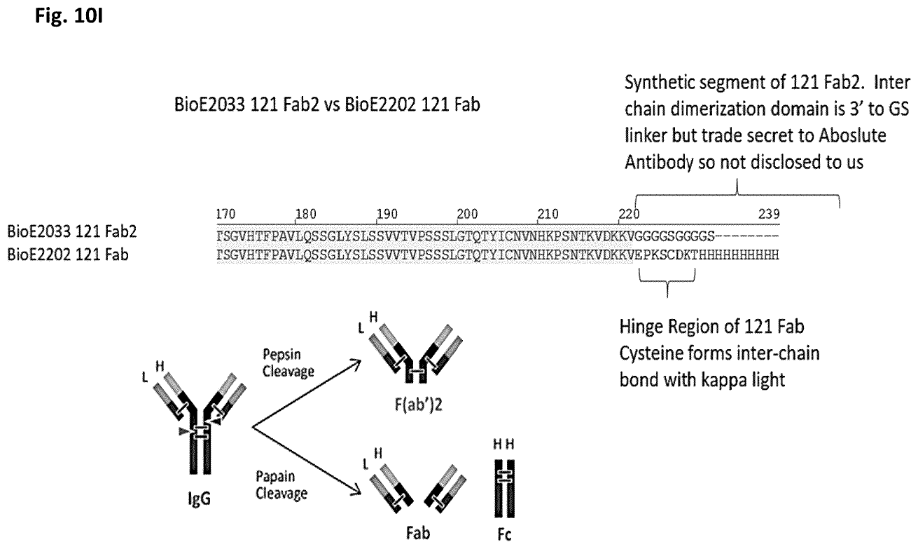

FIG. 13 depicts the results of the Promega CTLA4 Blockade bioassay done in triplicate and the read out is shown in Relative Luciferase Units, measured on a Glow-Max Navigator. Error bars shown are standard deviations. FIG. 14 depicts the readings rendered into ratios for ipilimumab and BioE2052. The activity titration curve for BioE2052 is displaced to 3× higher levels throughout the titration curve. The half-max activity concentration is between 8 and 40 nM. Arranging the V_{L} and V_{H} domains into a diabody configuration has led to both an unpredicted increase in the absolute activity in the Promega CTLA4 blockade assay and a decrease in the half max concentration. Such significant increase in the potency and specific activity was unpredicted, and exceeds the effect of mere avidity.

The covalent arrangement of Ipi and 121 VH and VL domains resulted in a diabody with unpredicted and unprecedented biological activity in the CTLA4 blockade assay. As described above, FIG. 14 shows the results expressed as the ratio of the relative luciferase units divided by the background no test agent relative luciferase unit. This ratio measurement allowed comparisons between and within the assays. The error bars are standard deviations. The assays were done in triplicate.

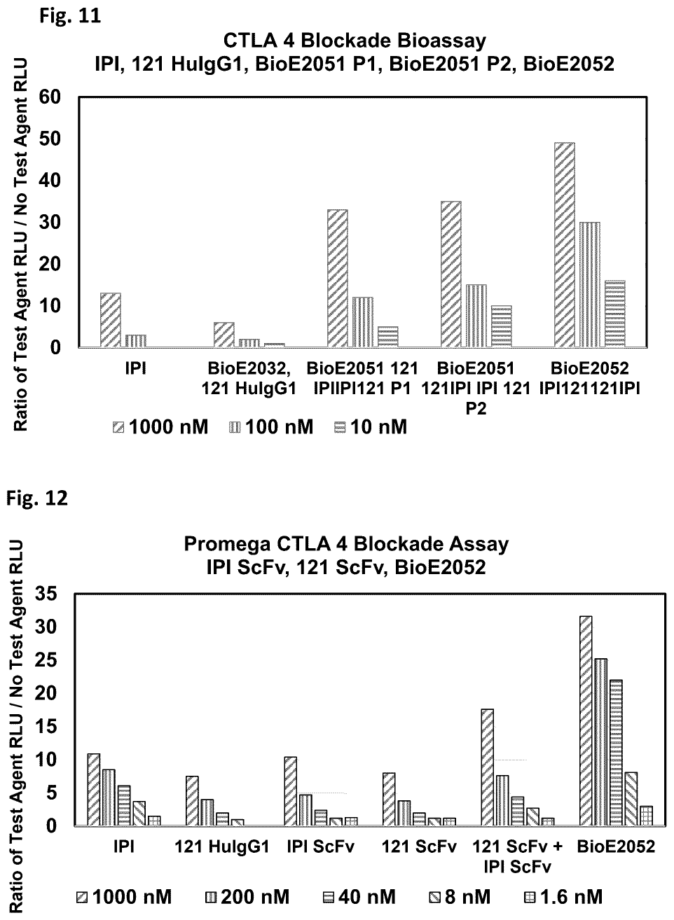

FIG. 15 is same as FIG. 13 but expressed as the ratios with the error bars. This is similar to FIG. 14, but with BioE2032 (BioE2032=121 HuIgG1) included.

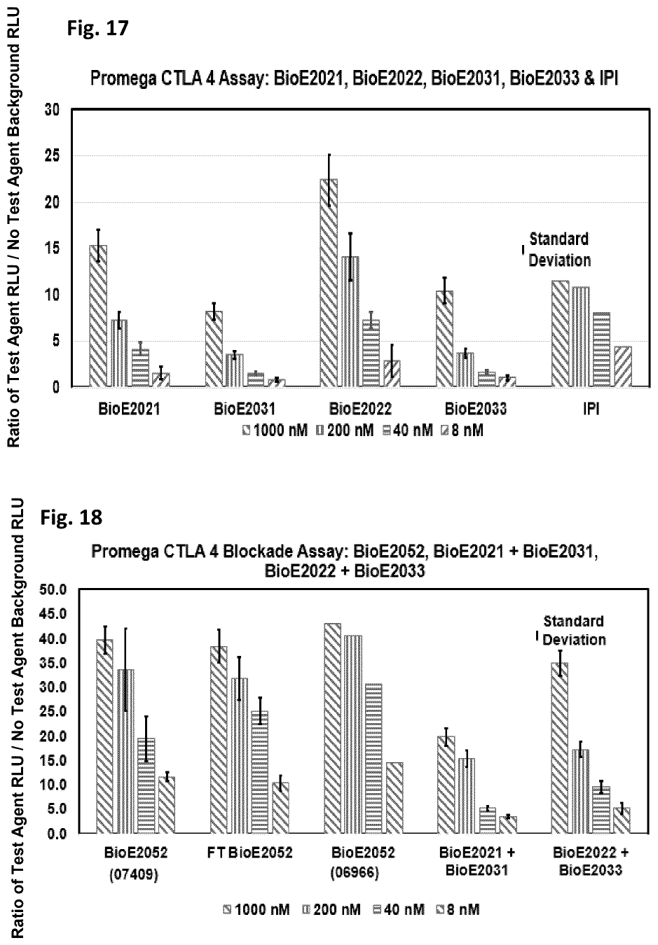

FIG. 18 shows the CTLA4-blocking activity of the anti-CTLA4 diabody (BioE2052), a combination of BioE2021 (Ipi scFv)+BioE2031 (121 scFv), and a combination of BioE2022 (Ipi F(ab′)2)+BioE2033 (121 F(ab′)2). The two samples on the right of the graph are: BioE2021+BioE2031 (mixture of Ipi ScFv+121 ScFv, each at 1000 nM, 200 nM, 40 nM and 8 nM) and BioE2022+BioE2033 (mixture of Ipi F(ab′)2+121 F(ab′)2). This assay was done in triplicate. The three BioE2052 samples on the left are BioE2052 (07409), which is a new lot of BioE2052, expressed and purified by Absolute Antibody™. It is from the Absolute Antibody™ project 07409. FT BioE2052 is a frozen thawed sample from the 07409 lot, demonstrating that freezing the recombinant protein at −80° C. and then thawing had marginal effects on its activity. Sample BioE2052 (06966) is the original material (Absolute Antibody™ project 06966). This has been stored at 4° C. and has maintained activity for at least 4 months. This material was assayed in duplicate.

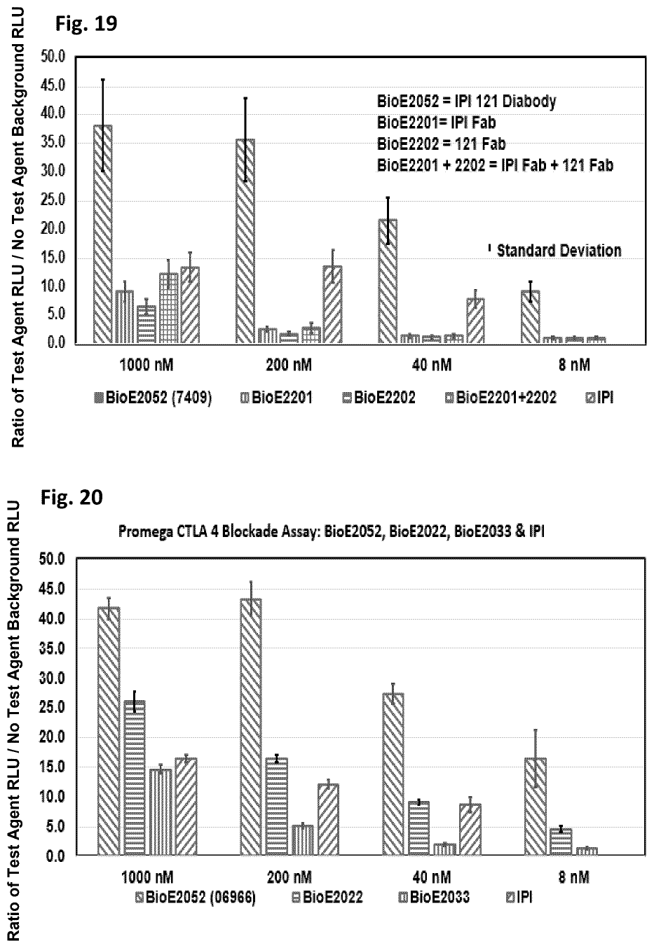

FIG. 19 shows the CTLA4-blocking activity of the anti-CTLA4 diabody (BioE2052), BioE2201 (Ipi Fab′), BioE2202 (121 Fab′), a combination of BioE2201 (Ipi Fab′)+BioE2202 (121 Fab′), and ipilimumab. Note the inferior level of activity of the mixture, which at best were additive of each component's activity as compared with the diabody BioE2052, which demonstrated synergism in activity.

In summary, activity is BioE2052>Ipi F(ab′)2+121 F(ab′)2>Ipi F(ab′)2>ipilimumab˜Ipi ScFv+121 ScFv>>Ipi Fab′+121 Fab′ (except at 1000 nM)>Ipi ScFv, 121 ScFv>Ipi Fab′, 121 Fab′.

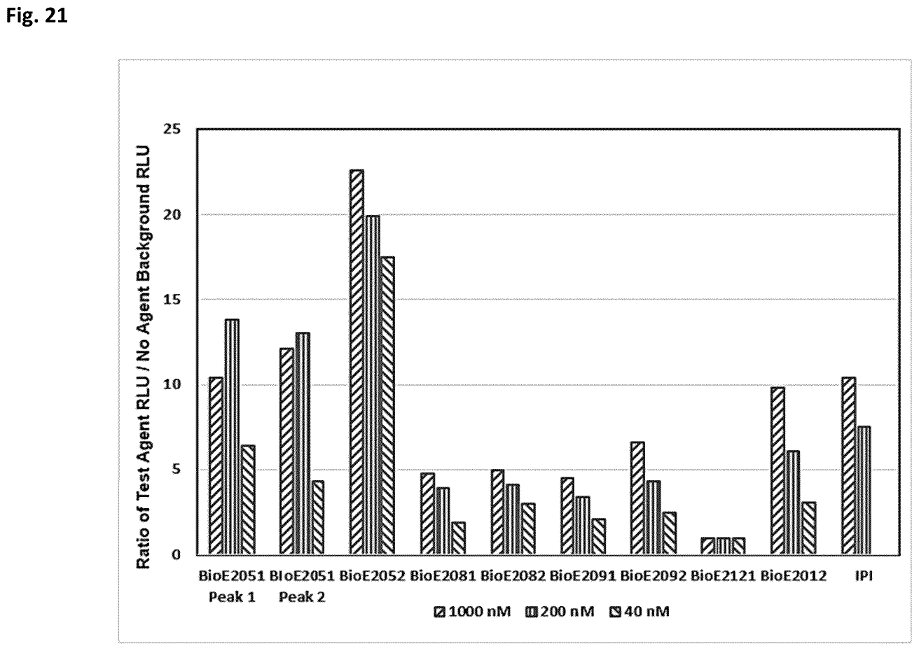

FIG. 20 shows the CTLA4-blocking activity of the anti-CTLA4 diabody (BioE2052 (06966)), BioE2022 (Ipi F(ab′)2), BioE2033 (121 F(ab′)2), and ipilimumab done in triplicate and data rendered in ratios.

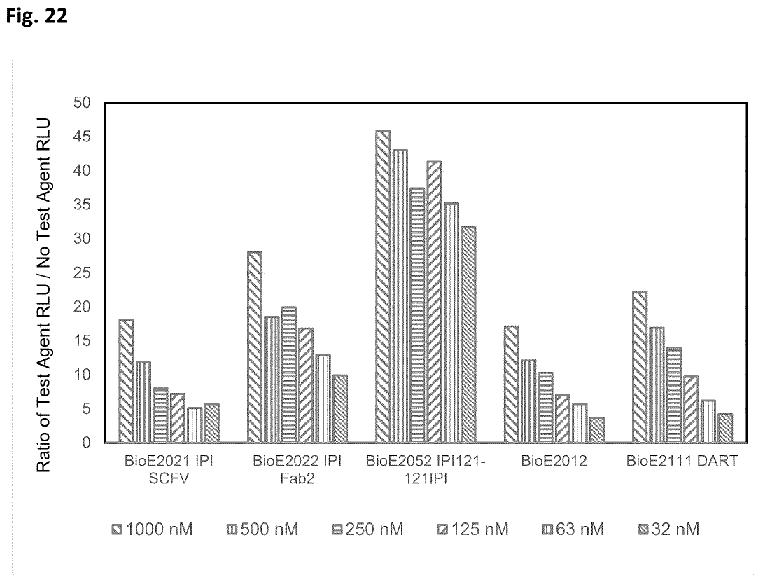

The CTLA4-blocking activities of the CTLA4-binding proteins BioE2051, BioE2052, BioE2081, BioE2082, BioE2091, BioE2092, BioE2121, BioE2012, and ipilimumab are shown in FIG. 21.

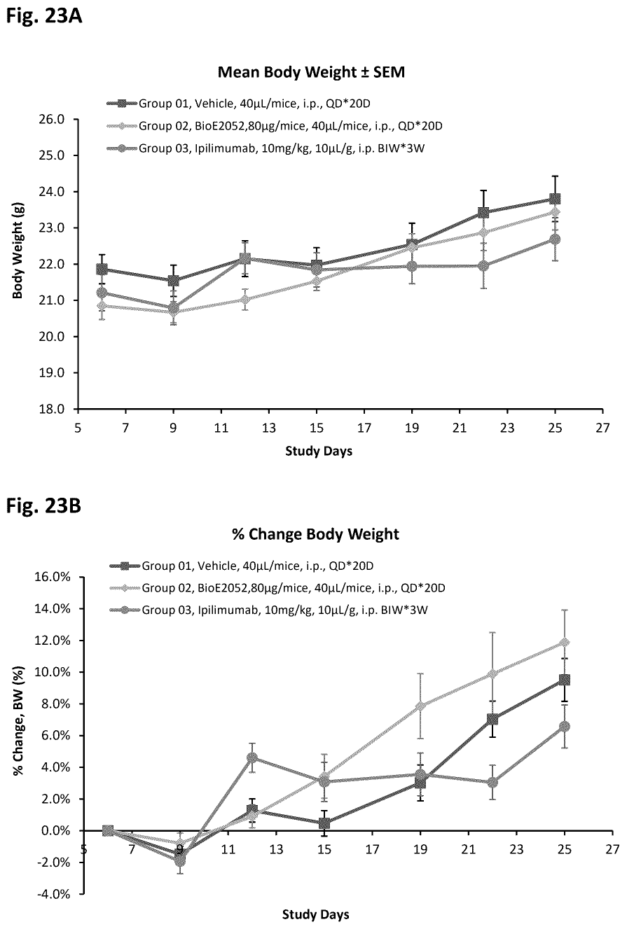

The CTLA4-blocking activity of the CTLA4-binding proteins BioE2021 Ipi ScFv, BioE2022 Ipi F(ab′)2, BioE2052 Ipi121-121Ipi, BioE2012, BioE2111 DART, and ipilimumab are shown in FIG. 22.

### Example 5: In Vivo Efficacy of BioE2052

To investigate the in vivo efficacy of BioE2052, a mouse model expressing a humanized CTLA4 and having a xenograft of liver tumor cells was dosed with ipilimumab or BioE2052. Specifically, xenografts of H22 mouse liver tumor cells were implanted into the right front flank of 6-9 week old female PD1/CTLA4 HuGEMM BALB/c mice (referred to herein as the H22 mouse model or H22 model). The mice were administered BioE2052, ipilimumab, or vehicle (i.e., saline) i.p. according to the treatment schedule shown in Table 5.

TABLE 5
Treatment Groups and Schedule

| | | | | Dosing | Dosing | | Dosing |
|---|---|---|---|---|---|---|---|
| | | | Dose level | Solution | Volume | | Frequency & |
| Group | No. | Treatment | (mg/kg) | (mg/ml) | (μL/g) | ROA | Duration |
| 1 | 10 | Vehicle | — | — | 40 | i.p. | QD × 20 days |
| | | | | | μL/mouse | | |
| 2 | 10 | BioE2052 | 80 | 2 | 40 | i.p. | QD × 20 days |
| | | | μg/mouse | | μL/mouse | | |
| 3 | 10 | Ipilimumab | 10 | 1 | 10 | i.p. | BIW × 3 weeks |
| Unassigned | 2 | BioE2052 | 80 | 2 | 40 | i.p. | Single dose |
| | | | μg/mouse | | μL/mouse | | |

### Cell Culture

The H22 tumor cell lines were maintained in vitro with RPMI1640 medium supplemented with 10% fetal bovine serum at 37° C., 5% CO₂. The tumor cells were subcultured twice weekly. The cells in an exponential growth phase were harvested and counted for tumor inoculation.

### Tumor Inoculation

Each mouse was inoculated subcutaneously in the right front flank region with H22 tumor cells (1×10⁶) in 0.1 ml of PBS for tumor development. The date of tumor cell inoculation was denoted as Day 0.

### Randomization

Two days before the randomization, two mice with a tumor volume (TV)>100 mm³ were injected with BioE2052 and observed daily before randomization. The two mice were observed and measured along with other mice in three groups after randomization.

The randomization started when the mean tumor size reached approximately 85 mm³ (Day 6). A total of 30 mice were enrolled in the study and allocated into 3 groups shown in Table 5, with 10 mice per group. Randomization was performed based on “Matched distribution” method (Study Director™ software, version 3.1.399.19).

### Observation and Data Collection

After tumor inoculation, the animals were checked daily for morbidity and mortality. During routine monitoring, the animals were checked for any effects of tumor growth and treatments on behavior such as mobility, food and water consumption, body weight gain/loss (Body weights would be measured twice per week after randomization), eye/hair matting and any other abnormalities. Mortality and observed clinical signs were recorded for individual animals in detail.

Tumor volumes were measured twice per week after randomization in two dimensions using a caliper, and the volume was expressed in mm³ using the formula: V=(L×W×W)/2, where V is tumor volume, L is tumor length (the longest tumor dimension) and W is tumor width (the longest tumor dimension perpendicular to L). Dosing as well as tumor and body weight measurements were conducted in a Laminar Flow Cabinet for 20 days (Day 6 to Day 25).

The body weights and tumor volumes were measured by using Study Director™ software (version 3.1.399.19).

### Statistical Analysis

To compare tumor volumes of different groups at a pre-specified day, Bartlett's test was used to validate the assumption of homogeneity of variance across all groups. When the p-value of Bartlett's test is >0.05, one-way ANOVA was performed to test overall equality of means across all groups. If the p-value of the one-way ANOVA is <0.05, post hoc testing was performed by running Tukey's HSD (honest significant difference) tests for all pairwise comparisons and Dunnett's tests for comparing each treatment group with the vehicle group. When the p-value of Bartlett's test was <0.05, the Kruskal-Wallis test was performed to test overall equality of medians among all groups. If the p-value the Kruskal-Wallis test was <0.05, post hoc testing was performed by running Conover's non-parametric test for all pairwise comparisons or for comparing each treatment group with the vehicle group, both with single-step p-value adjustment. All statistical analyses were done in R-a language and environment for statistical computing and graphics (version 3.3.1). All tests were two-sided unless otherwise specified, and p-values of <0.05 were regarded as statistically significant.

For survival analysis, the survival time was analyzed by the Kaplan-Meier method. The survival time was defined as the time from the day of randomization until animal death or ethical endpoint. For each group, the median survival time (MST) and the increased in life-span (ILS) were calculated. The Kaplan-Meier curves were also constructed for each group and the log-rank test was used to compare survival curves between groups.

### Results

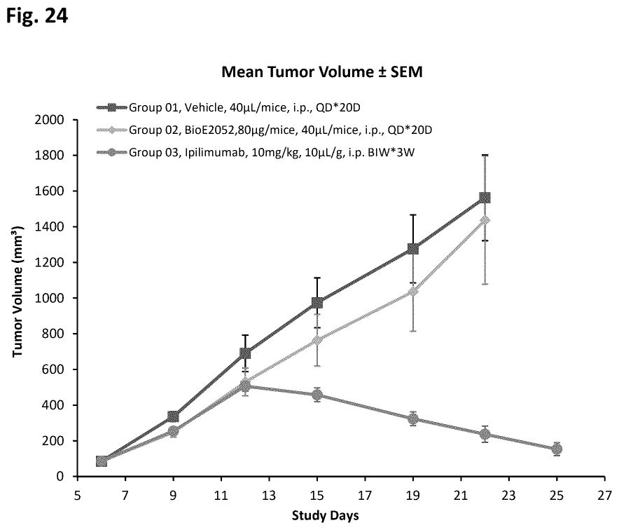

The results of body weights and body weight changes at different time points are shown in FIGS. 23A and 23B, respectively.

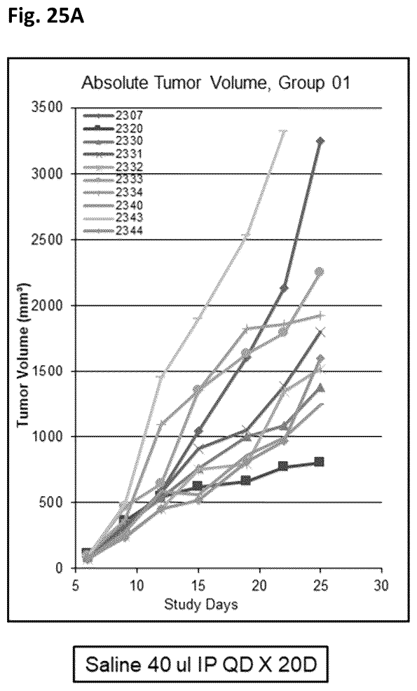

Tumor volume was also assessed during the study. Mean tumor growth curves at different time points are shown in FIG. 24. The mean tumor growth curve stopped at the first animal sacrifice due to tumor size in each group. During this study, 3 mice were sacrificed due to a TV>3000 mm³. One mouse that was not assigned was also sacrificed due to a TV>3000 mm³ (not shown).

Tumor growth inhibition observed in BioE2052-treated and ipilimumab-treated mice is summarized in Table 6 below. The data was used on the specific day when the first mouse was sacrificed due to tumor size.

TABLE 6
Antitumor Activity of BioE2052 and Ipilimumab in H22 Model

| | | Tumor Size | T/C | TGI | |
|---|---|---|---|---|---|
| | Treatment | (mm³) ^{a} on | (%) on | (%) on | P |
| Group | Description | Day 22 | Day 22 | Day 22 | value ^{b} |
| 1 | Vehicle, | 1562.45 ± 240.26 | — | — | — |
| | 40 μL/mouse, | | | | |
| | i.p., QD | | | | |
| 2 | BioE2052, | 1436.65 ± 359.16 | 92 | 8 | 0.568 |
| | 80 μg/mouse, | | | | |
| | i.p., QD | | | | |
| 3 | Ipilimumab, | 236.93 ± 45.47 | 15 | 85 | <0.001 |
| | 10 mg/kg, | | | | |
| | i.p., BIW | | | | |

Note:
^{a} Mean ± SEM;
^{b} compared with group 1 tumor volume on Day 22.
In this study, the mean tumor size of the vehicle control mice reached 1562.45 mm³ on Day 22 post randomization.

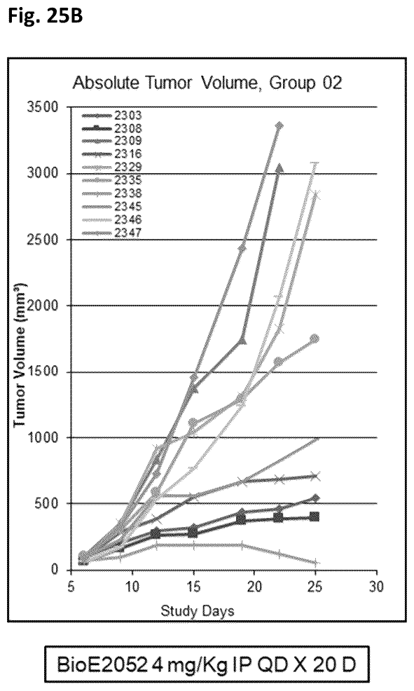

BioE2052 was administered at 80 μg/mouse, QD, which produced slight anti-tumor efficacy in the H22 model, with a TGI value of 8% on Day 22. However, no statistically significant difference (P>0.05) was observed when compared with vehicle control group. Importantly, a therapeutic effect was observed in the BioE2052-treated group as three of the ten mice in this group exhibited tumor regression or stabilization (FIGS. 25A-25C).

Ipilimumab administered at 10 mg/kg, BIW, produced significant anti-tumor efficacy in the H22 model, with TGI value of 85% on Day 22, yielding a statistically significant difference (p<0.05) when compared with vehicle control group. Two ipilimumab-treated animals died, but this mortality rate is often observed in ipilimumab-treated subjects.

The β half-life of BioE2052 was about 30 minutes after a rapid drop (short a phase) upon i.p. injection. After 24 hours, only about 1% of the initial material was found in the mouse circulation.

### Example 6: BioE2052-CTLA4 Interaction

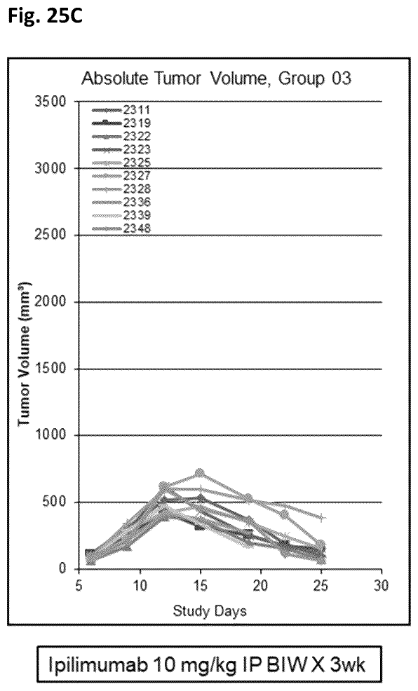

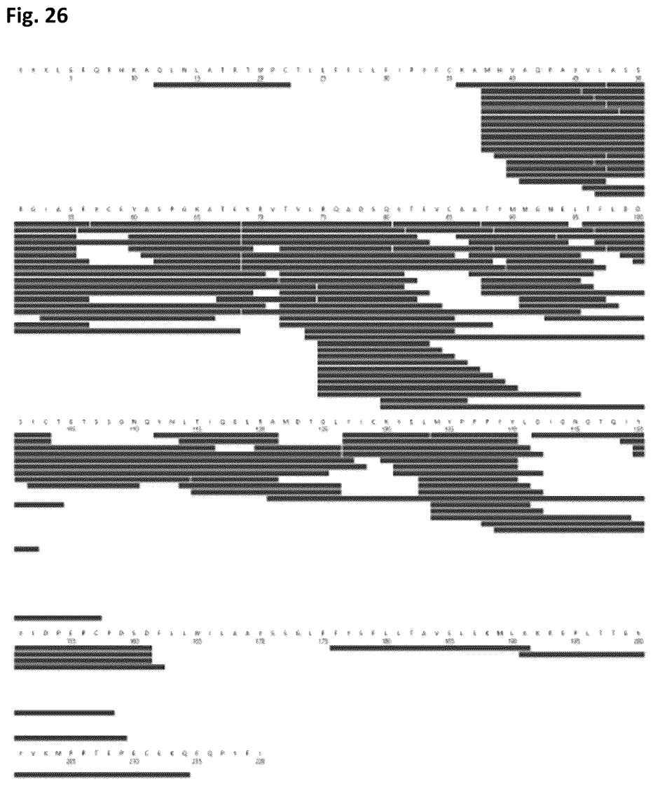

To better understand the binding of BioE2502 to CTLA4, hydrogen-deuterium exchange mass spectrometry (HDX) was performed. HDX identifies binding regions between to interacting peptides as the bound regions of both are protected from deuterium exchange. CTLA4 and the antibody were combined at 1:1 ratio without dilution in order to drive occupancy (the more antibody present the better). Data was acquired for 0, 10 sec, 5 min, and 30 min. HDX generates a heat map in which the degree of H to D exchange is indicated by the color scheme. In this case, the stronger the blue hue, the more the protection in the presence of antibody. The epitopes on CTLA4 that are bound by BioE2502 were identified (FIGS. 26 and 27).

TABLE 7
Summary of Hydrogen-Deuterium Exchange Experiments

| | Protection | Protection |
|---|---|---|
| | increases with | decreases with |
| Complex | antibody present | antibody present |
| CTLA4 + | Leu13 to Cys22 | Weakly Val151 |
| BioE2502 | Gln80 to Ala86 | to Asp162 |
| | Ile128 to Met134 | |
| | weakly Ala61 | |
| | to Ser80 | |

### Example 7: BioE2052-IgG1 Fc Fusion Proteins

In an effort to increase half-life and stability of BioE2052, constructs were generated that encode BioE2052 fused to a conventional IgG1 Fc or to an IgG1 Fc with an LALAPG mutation. This mutation blocks BioE2052 binding to the Fcγ receptor, thus eliminating antibody-dependent cellular cytotoxicity (ADCC). This overcomes a significant shortfall of ipilimumab, which may be exclusively a Treg depletory antibody rather than a CTLA4-blocking antibody.

The addition of an albumin-binding protein to BioE2052 was also examined. Specifically, the carboxy end of BioE2052 was fused to the amino end of mouse albumin. The SEC profile of BioE2052 fused to mouse albumin showed 2 peaks. The two peaks were combined in these preliminary experiments but can be collected and individually analyzed.

### Example 8: Antigen-Binding Proteins Fused to an IgG Fc Peptide or an Albumin-Binding Proteins Display Increases Stability

Two monoclonal anti-CTLA4 antigen-binding proteins (BioE2420 and BioE2430 (SEQ ID NOs: 61 and 63, respectively)) were generated using commercially available methods (absoluteantibody.com/our-technology/our-recombinant-technology/). BioE2420 comprises the amino acid sequence of BioE2052 (SEQ ID NO: 24) with an Fc peptide fused to the carboxy terminal of the BioE2052 peptide. BioE2430 comprises the amino acid sequence of BioE2052 (SEQ ID NO: 24) with an albumin peptide fused to the carboxy terminal of the BioE2052 peptide.

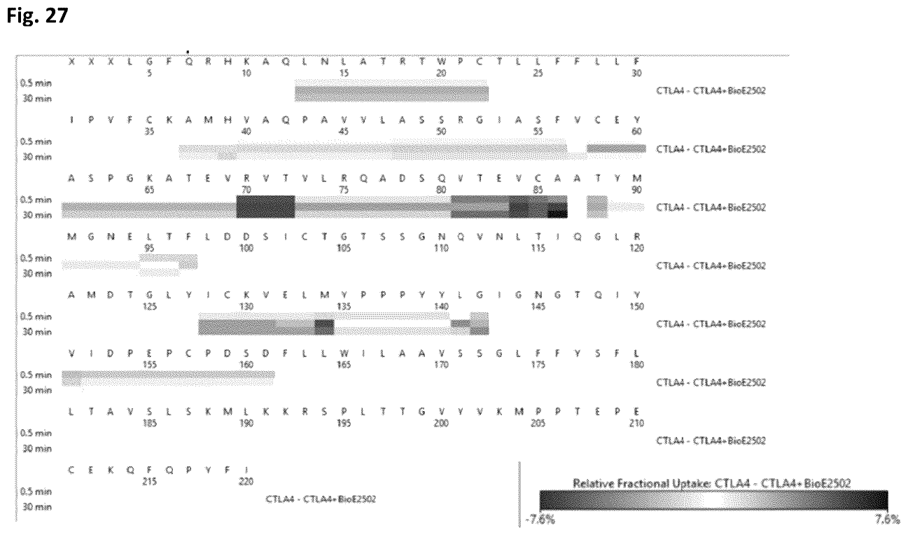

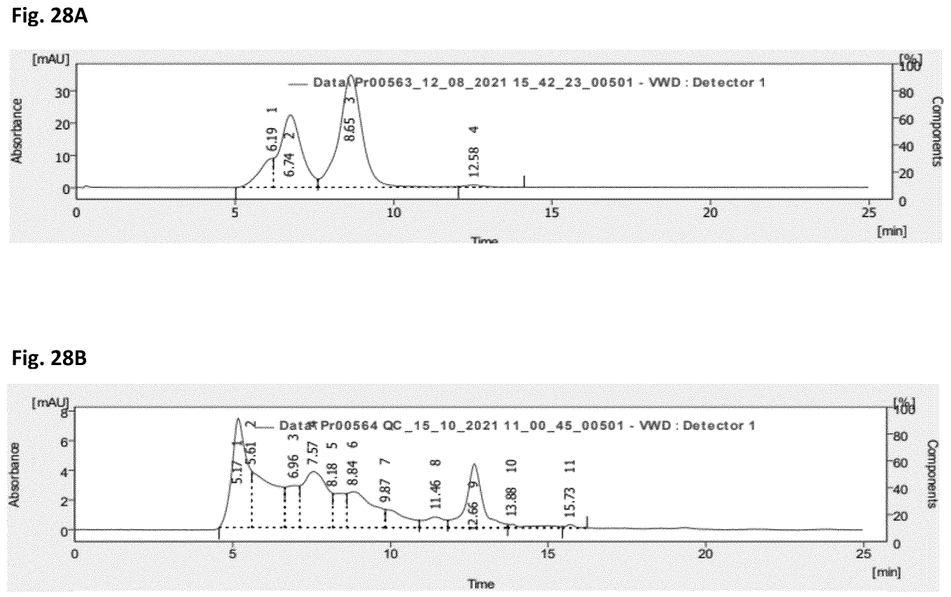

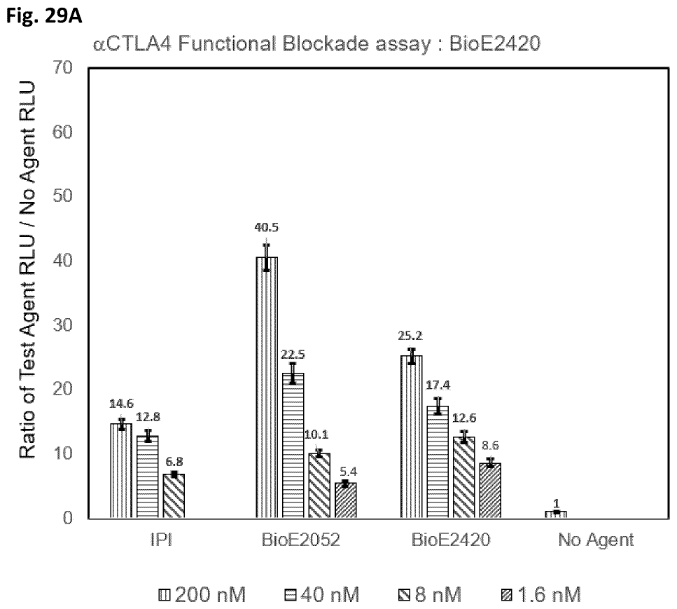

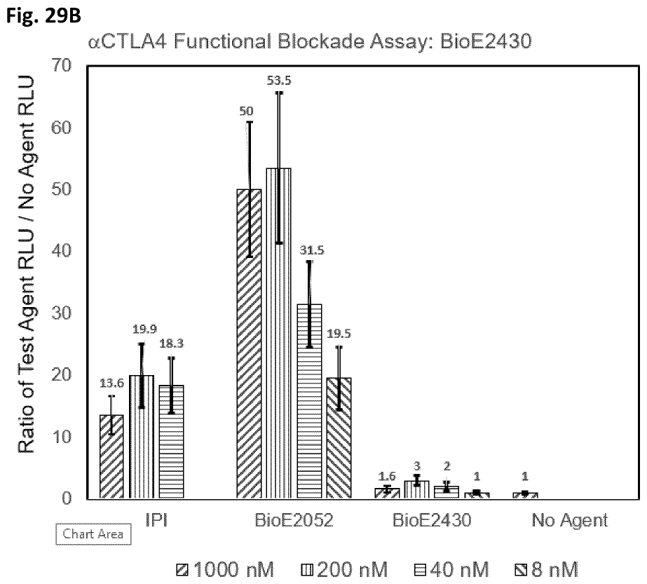

Monomers were purified by size exclusion HPLC (SEC-HPLC) (FIGS. 28A and 28B). The activity of these purified monomers was assessed using the CTLA4 Functional Blockade Assay (Promega; see example 7). As shown in FIG. 29A, BioE2420 has less anti-CTLA4 activity relative to BioE2052, but more than Ipi, which acts in a manner other than by blocking CTLA4. BioE2430 has very little activity, roughly equivalent to the negative control (FIG. 29B). These results show that antigen-binding proteins modified to have another protein domain fused to the carboxy terminus can have anti-CTLA4 activity.

### Example 9

To improve or modify the pharmacokinetic (PK) properties of antigen-binding proteins, antigen-binding domains (e.g., BioE2052) are modified (e.g., fused) to comprise a PK modulator. The modified antigen-binding protein is administered to H22 model mice according to the schedule shown in Table 6. Blood samples are drawn at regular intervals and assayed for the presence of the modified antigen-binding protein to determine the bioavailability of the modified antigen-binding protein. Results are compared to those observed in H22 mice that are administered vehicle (e.g., saline), those that are administered ipilimumab, and those that are administered only the antigen-binding protein (i.e., no Fc domain). Results show an improvement in the bioavailability of the modified antigen-binding protein relative to the normal control group and the group that are administered only the antigen binding protein. Additionally, survivability is improved in mice administered the modified antigen-binding protein relative to the normal control group and the group that are administered only the antigen binding protein.

Tumor volume and growth are measured in the H22 mice that are administered the modified antigen-binding protein and are compared to tumor volume and growth observed in H22 mice that are administered only vehicle (e.g., saline), H22 mice that are administered ipilimumab, and H22 mice that are administered only the antigen-binding protein. Results show an improvement in tumor volume and growth in mice receiving the modified antigen-binding protein relative to the normal control group and the group administered only the antigen binding protein.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the present invention described herein. Such equivalents are intended to be encompassed by the following claims.

1. An engineered antigen-binding protein comprising antigen-binding domains that specifically binds to Epitope 1 defined by the residues ¹³⁴MYPPPY¹³⁹ (SEQ ID NO: 1) and Epitope 2 defined by the residues ⁶⁵SICT⁶⁸ (SEQ ID NO: 2) of cytotoxic T-lymphocyte-associated antigen-4 (CTLA4), or
an engineered antigen-binding protein that specifically binds to CTLA4 and lacks the CH2 domain and/or a CH3 domain of the constant region of an antibody.

2. The engineered antigen-binding protein of claim 1, wherein the engineered antigen-binding protein comprises:
a) a heavy chain variable domain (VH) sequence with at least or about 85% identity to a VH sequence selected from the group consisting of the VH sequences listed in Table 3; and/or
b) a light chain variable domain (VL) sequence with at least or about 85% identity to a VL sequence selected from the group consisting of the VL sequences listed in Table 3.

3. The engineered antigen-binding protein of claim 1, wherein the engineered antigen-binding protein comprises a sequence with at least or about 85% identity to the sequence selected from SEQ ID NOs: 9, 10, 12, 14, 15, 17, 19, 20-24, 27, 28, 29-54, and 59, 61, 63, 65, 67, and 71, optionally wherein the engineered antigen-binding protein comprises a sequence with at least or about 85% identity to the sequence selected from:
a) SEQ ID NOs: 9 and 10;
b) SEQ ID NOs: 9 and 14;
c) SEQ ID NOs: 9 and 17;
d) SEQ ID NOs: 12 and 10;
e) SEQ ID NOs: 12 and 14;
f) SEQ ID NOs: 12 and 17;
g) SEQ ID NOs: 15 and 10;
h) SEQ ID NOs: 15 and 14;
i) SEQ ID NOs: 15 and 17;
j) SEQ ID NOs: 9, 10, 15, and 17;
k) SEQ ID NOs: 12, 14, 15, and 17;
l) SEQ ID NOs: 21 and 22;
m) SEQ ID NOs: 27 and 28;
n) SEQ ID NOs: 31, 32, and 33;
o) SEQ ID NOs: 34, 35, and 36;
p) SEQ ID NOs: 37, 38, and 39;
q) SEQ ID NOs: 40, 41, and 42;
r) SEQ ID NOs: 43 and 44;
s) SEQ ID NOs: 45 and 46;
t) SEQ ID NOs: 47, 48, and 49;
u) SEQ ID NOs: 50, 51, and 52;
v) SEQ ID NOs: 53 and 54;
w) SEQ ID NO: 59;
x) SEQ ID NO: 61;
y) SEQ ID NO: 63;
z) SEQ ID NOs: 65 and 67; and
aa) SEQ ID NOs: 69 and 71.

4-7. (canceled)

8. The engineered antigen-binding protein of claim 1, wherein
(a) the engineered antigen-binding protein is selected from an antibody, Fv, F(ab′)2, Fab′, dsFv, scFv, sc(Fv)2, half antibody-scFv, tandem scFv, tandem biparatopic scFv, Fab/scFv-Fc, tandem Fab′, single-chain diabody, tandem diabody (TandAb), Fab/scFv-Fc, heterodimeric Fab/scFv-Fc, heterodimeric scFv-Fc, heterodimeric IgG (CrossMab), DART, and diabody;
(b) the engineered antigen-binding protein comprises an immunoglobulin heavy chain constant domain selected from the group consisting of IgG, IgG1, IgG2, IgG2A, IgG2B, IgG3, IgG4, IgA, IgM, IgD, and IgE constant domains;
(c) the engineered antigen-binding protein comprises an Fc domain, optionally wherein
(i) the Fc domain is heterologous relative to the antigen-binding domain,
(ii) the Fc domain is an IgG1 Fc,
(iii) the Fc domain does not bind to one or more Fc receptors, optionally wherein the Fc receptor is a neonatal Fc receptor;
(iv) the Fc domain comprises a mutation that inhibits binding of the antigen-binding protein to the neonatal Fc receptor, optionally wherein the mutation is a LALAPG mutation; or
(d) the engineered antigen-binding protein does not comprise (i) an Fc domain, or (ii) the CH2 domain and/or CH3 domain of the constant region of an antibody.

9-23. (canceled)

24. The engineered antigen-binding protein of claim 1, wherein engineered antigen-binding protein lacks the CH2 domain and/or a CH3 domain of the constant region of an antibody, and:
(a) the engineered antigen-binding protein is F(ab′)2;
(b) the engineered antigen-binding protein comprises the sequence of SEQ ID NOs: 21 and 22;
(c) the engineered antigen-binding protein comprises the sequence of SEQ ID NOs: 27 and 28; or
(d) the engineered antigen-binding protein binds two epitopes: Epitope 1 defined by the residues ¹³⁴MYPPPY¹³⁹ (SEQ ID NO: 1) and Epitope 2 defined by the residues ⁶⁵SICT⁶⁸ (SEQ ID NO: 2) of CTLA4.

25-27. (canceled)

28. The engineered antigen-binding protein of claim 24, wherein the engineered antigen-binding protein comprises at least two different VH domains and at least two different VL domains, optionally wherein the engineered antigen-binding protein is a DART or a diabody.

29. (canceled)

30. The engineered antigen-binding protein of claim 28, wherein the engineered antigen-binding protein is a DART or a diabody, and the DART or the diabody comprises, in the N-terminal to C-terminal direction:
a) a first VH;
b) a first VL;
c) a second VH; and
d) a second VL;
wherein the first VH and the first VL are linked by a Linker A; the first VL and the second VH are linked by a Linker B; and the second VH and the second VL are linked by a Linker C.

31. The engineered antigen-binding protein of claim 30, wherein
(a) the Linker A and/or Linker B comprise a peptide sequence of (GlyGlySer)n, wherein n=1-20 (SEQ ID NO: 74), optionally wherein the n=0, 1, 2, or 3, optionally wherein the n=3;
(b) the Linker C comprises a peptide sequence of (GlyGlySer)n, wherein n=1-20 (SEQ ID NO: 74), optionally wherein
(i) the n is greater than 4;
(ii) n=0, 1, 2, 3, or 4; or
(iii) n=3;
(c) each of the Linker A, Linker B, and Linker C comprises (GlyGlySer)n, wherein n=3 (SEQ ID NO: 75); or
(d) the first VH and the second VL bind the Epitope 1 (SEQ ID NO: 1); and the first VL and second VH bind the Epitope 2 (SEQ ID NO: 2).

32-39. (canceled)

40. The engineered antigen-binding protein of claim 30, wherein
(a) the first VH, first VL, second VH, and second VL comprise the sequence with at least or about 85% identity to SEQ ID NO: 15, SEQ ID NO: 14, SEQ ID NO: 12, and SEQ ID NO: 17, respectively;
(b) the engineered antigen-binding protein comprises the sequence with at least or about 85% identity to SEQ ID NO: 24 (BioE2052);
(c) the first VH and the second VL bind the Epitope 2 (SEQ ID NO: 2); and the first VL and second VH bind the Epitope 1 (SEQ ID NO: 1);
(d) the first VH, first VL, second VH, second VL comprise the sequence with at least or about 85% identity to SEQ ID NO: 12, SEQ ID NO: 17, SEQ ID NO: 15, and SEQ ID NO: 14, respectively; or
(e) the engineered antigen-binding protein comprises the sequence with at least or about 85% identity to SEQ ID NO: 23 (BioE2051).

41-48. (canceled)

49. The engineered antigen-binding protein of claim 1, wherein
(a) the CTLA4 is a human CTLA4;
(b) the engineered antigen-binding protein is chimeric, humanized, composite, murine, or human;
(c) the engineered antigen-binding protein further comprises a peptide tag (e.g., His6 tag) (SEQ ID NO: 78) and/or a leader sequence;
(d) the engineered antigen-binding protein is conjugated or detectably labeled, optionally wherein the engineered antigen-binding protein is PEGylated;
(e) the engineered antigen-binding protein self-dimerizes; or
(f) the engineered antigen-binding protein:
(i) blocks the interaction between CTLA4 and its ligands (e.g., CD80 (B7-1) and CD86 (B7-2)); and/or
(ii) increases the interleuken-2 (IL-2) expression by the T cells.

50-54. (canceled)

55. An isolated nucleic acid molecule that encodes the engineered antigen-binding protein of claim 1, or a vector comprising the isolated nucleic acid.

56. (canceled)

57. A host cell which
(a) comprises an isolated nucleic acid encoding the engineered antigen-binding protein of claim 1;
(b) comprises a vector comprising the isolated nucleic acid of (a); or
(c) expresses the engineered antigen-binding protein of claim 1.

58. A pharmaceutical composition comprising:
(a) the engineered antigen-binding protein of claim 1;
(b) an isolated nucleic acid encoding the engineered antigen-binding protein of claim 1;
(c) a vector comprising the isolated nucleic acid of (b); or
(d) a host cell comprising any one of (a)-(c).

59. A kit comprising at least one engineered antigen-binding protein of claim 1.

60. A method of producing at least one engineered antigen-binding protein of claim 1, wherein the method comprises the steps of: (i) culturing a host cell comprising a nucleic acid comprising a sequence encoding at least one engineered antigen-binding protein of claim 1 under conditions suitable to allow expression of said engineered antigen-binding protein; and (ii) recovering the expressed engineered antigen-binding protein.

61. A method of preventing or treating a subject afflicted with cancer or a method of reducing proliferation of a cancer cell in a subject in need thereof, the method comprising administering to the subject at least one engineered antigen-binding protein of claim 1 or a pharmaceutical composition comprising the at least one engineered antigen-binding protein.

62. (canceled)

63. The method of claim 61, wherein
(i) the at least one engineered antigen-binding protein or the pharmaceutical composition (a) reduces the number of proliferating cancer cells in the cancer; (b) reduces the volume or size of a tumor of the cancer; (c) increases the immune response against the cancer; and/or (d) activates the T cell;
(ii) the method further comprises comprising administering to the subject an additional cancer therapy, optionally wherein the additional cancer therapy is selected from the group consisting of immunotherapy, checkpoint blockade, cancer vaccines, chimeric antigen receptors, chemotherapy, radiation, target therapy, and surgery, optionally wherein the additional cancer therapy is nivolumab; or
(iii) the cancer is selected from pancreatic cancer, lung cancer, non-small cell lung cancer (NSCLC), malignant pleural mesothelioma, small cell lung cancer (SCLC), renal cell carcinoma (RCC), breast cancer, liver cancer, hepatocellular carcinoma, kidney cancer, skin cancer, melanoma (e.g., unresectable or metastatic melanoma), thyroid cancer, gall bladder cancer, head and neck cancer, stomach cancer, head and neck cancer, bladder cancer, urothelial carcinoma, Merkel cell cancer, colon cancer, colorectal cancer, intestinal cancer, ovarian cancer, cervical cancer, testicular cancer, esophageal cancer, buccal cancer, brain cancer, blood cancers, lymphomas (B and T cell lymphomas), mesothelioma, cutaneous squamous cell cancer, Hodgkin's lymphoma, B-cell lymphoma, and a malignant or metastatic form thereof.

64-67. (canceled)

68. A method of increasing an immune response in a subject, or a method of activating a T-cell in a subject, or a method of preventing or treating a disease of a condition characterized by aberrant expression or activity of a CTLA4 protein in a subject in need thereof, the method comprising administering to the subject at least one engineered antigen-binding protein of claim 1 or a pharmaceutical composition comprising the at least one engineered antigen-binding protein.

69-70. (canceled)

71. The method of claim 68, wherein the subject is afflicted with a cancer, autoimmune disease, infection, or inflammatory disease.
